(19) United States
(12) Patent Application Publication
(10) Pub. No.: US 2005/0136483 A1
(43) Pub. Date: Jun. 23, 2005

(54) Nanodevices employing combinatorial artificial receptors

(75) Inventor: Robert E. Carlson, Minnetonka, MN (US)

Correspondence Address: MERCHANT & GOULD PC, P.O. BOX 2903, MINNEAPOLIS, MN, 55402-0903, US

(73) Assignee: RECEPTORS LLC, CHASKA, MN (US)

(21) Appl. No.: 10/934,879

(22) Filed: Sep. 3, 2004

Related U.S. Application Data
(60) Provisional application No. 60/499,752, filed on Sep. 3, 2003.
(60) Provisional application No. 60/500,081, filed on Sep. 3, 2003.
(60) Provisional application No. 60/499,776, filed on Sep. 3, 2003.
(60) Provisional application No. 60/499,867, filed on Sep. 3, 2003.
(60) Provisional application No. 60/499,965, filed on Sep. 3, 2003.
(60) Provisional application No. 60/499,975, filed on Sep. 3, 2003.
(60) Provisional application No. 60/526,511, filed on Dec. 2, 2003.
(60) Provisional application No. 60/526,699, filed on Dec. 2, 2003.
(60) Provisional application No. 60/526,703, filed on Dec. 2, 2003.
(60) Provisional application No. 60/526,708, filed on Dec. 2, 2003.
(60) Provisional application No. 60/527,190, filed on Dec. 2, 2003.

(51) Int. Cl. H01L 21/00; G01N 33/53

(52) U.S. Cl. 435/7.1; 438/1

(57) ABSTRACT

The present invention includes nanodevices employing combinatorial artificial receptors and methods for making and using the same. In an embodiment the invention includes a method of adhering components together. In an embodiment, the invention includes a device including a first component adhered to a second component via a binding pair of artificial receptors. In an embodiment, the invention includes an agent delivery device having a capsule, and an active agent. In an embodiment, the invention can include a detection device having a magnetic particle and an artificial receptor disposed thereon. In an embodiment, the invention can include a detection device having a quantum dot and an artificial receptor disposed on the quantum dot. In an embodiment, the invention includes a detection device having first particles and second particles that aggregate in the present of a target ligand. In an embodiment, the invention includes a detection device having a cantilever and an artificial receptor disposed thereon. In an embodiment, the invention can include a detection device having a substrate and an artificial receptor disposed thereon. In an embodiment, the invention can include a device for selective removal of a target component including a substrate and an artificial receptor disposed thereon.

### CROSS REFERENCE TO RELATED APPLICATIONS

The present application claims priority to U.S. Provisional Patent Application Nos. 60/499,752, 60/500,081, 60/499,776, 60/499,867, 60/499,965, and 60/499,975 each filed Sep. 3, 2003; and 60/526,511 60/526,699, 60/526,703, 60/526,708, and 60/527,190 each filed Dec. 2, 2003. Each of these patent applications is incorporated herein by reference in its entirety.

### FIELD OF THE INVENTION

The present invention relates to systems, devices, and articles on a micro- or nano-scale utilizing artificial receptors, and methods of making and using them. More specifically, the present invention relates to micro- or nanodevices employing combinatorial artificial receptors and methods for making and using the same.

### BACKGROUND OF THE INVENTION

Nanodevices are structures having dimensions measured in nanometers. Nanotechnology is a field associated with formation of nanodevices, and is a growing field expected to make significant impacts in diverse subject areas, including, for example, biology, chemistry, computer science and electronics. Even though the field is called nanotechnology, it covers many devices and systems that are many nanometers or some micrometers in size.

Nanodevices include, for example, quantum dots and nanowires. A quantum dot (or semiconductor nanocrystal) is a particle of matter in which addition or removal of an electron changes its properties in some useful way. A nanowire is a thin filament having a width less than or equal to about 200 angstroms, and frequently less than or equal to about 50 angstroms.

Many nanodevices depend on one component adhering to another, either for assembly of the nanodevice or for proper functioning of the nanodevice. Other nanodevices can be benefit from being able to specifically bind or adhere to another object, cell, or molecule. For example, some nanodevices should have components adhered together in a specific manner in order for the nanodevice to be assembled properly. As another example, some nanodevices should be able be able to adhere to a specific substrate in order to be operational. Other nanodevices can be benefit from being able to specifically bind or adhere to another object, cell, or molecule.

Although various techniques have been developed for forming nanodevices, and adhering nanocomponents together and to substrates or objects, there remains a need to develop methods and systems for efficient or specific adherence or binding.

### SUMMARY OF THE INVENTION

The present invention relates to nanodevices employing combinatorial artificial receptors and methods for making and using the same. In an embodiment the invention includes a method of adhering components together. The method includes disposing a first artificial receptor on a first component, wherein the first artificial receptor includes a plurality of building blocks coupled to the first component, and wherein the first artificial receptor is known to having binding affinity for a second component. The method also includes allowing the artificial receptor to bind to the second component. In an embodiment, the invention includes a device including a first component and a second component. The device can also include a first binding pair of artificial receptors including a first artificial receptor and a second artificial receptor. The first artificial receptor can be disposed on the first component and the second artificial receptor can be disposed on the second component. In an embodiment, the first component can be adhered to the second component via the first binding pair. In an embodiment, the invention includes an agent delivery device having a capsule, and an active agent, wherein the active agent is disposed within the capsule. An artificial receptor can be disposed on the capsule, wherein the artificial receptor is known to have binding affinity for a target ligand. In an embodiment, the invention can include an agent delivery device having a nanotube, an active agent disposed on the nanotube, and a cap disposed on the nanotube having an open position and a closed position. An artificial receptor can be disposed on the cap, wherein the artificial receptor has a binding affinity for the nanotube that can be overcome by a release compound. In an embodiment, the cap is in the closed position when the artificial receptor is bound to the nanotube. In an embodiment, the invention can include a detection device having a magnetic particle and an artificial receptor disposed on the magnetic particle. In an embodiment, the invention can include a detection device having a quantum dot and an artificial receptor disposed on the quantum dot. In an embodiment, the invention can include a detection device having a plurality of first particles and a plurality of first artificial receptors disposed on the first particles. In an embodiment, the first artificial receptors can have binding affinity for a first part of a target ligand. The detection device can also include a plurality of second particles and a plurality of second artificial receptors disposed on the second particles, the second artificial receptors known to have binding affinity for a second part of a target ligand. In an embodiment, the first particles and the second particles aggregate in the present of the target ligand. In an embodiment, the invention can include a detection device having a cantilever and an artificial receptor disposed on the cantilever. In an embodiment, the invention can include a detection device having a substrate and an artificial receptor disposed on the substrate. The substrate can include a nanowire. The substrate can include a nanowire field effect transistor. The substrate can also include a nanotube. In an embodiment, the conductance of the substrate can change when the target ligand is bound to the artificial receptor. In an embodiment, the invention can include a device for selective removal of a target component including a substrate and an artificial receptor disposed on the substrate.

### BRIEF DESCRIPTION OF THE FIGURES

FIG. 1 schematically illustrates two-dimensional representations of an embodiment of a receptor according to the present invention that employs 4 different building blocks to make a ligand binding site.

FIG. 2 schematically illustrates two and three-dimensional representations of an embodiment of a molecular configuration of 4 building blocks, each building block including a recognition element, a framework, and a linker coupled to a support (immobilization/anchor).

FIG. 3 schematically illustrates an embodiment of the present methods and artificial receptors employing shuffling and exchanging building blocks.

FIG. 4 is a flow chart illustrating a process for identifying and working receptors and disposing them on a component.

FIG. 5 is a flow chart illustrating a process for making pairs of artificial receptors.

FIG. 6 is a schematic diagram of a plurality of nano-scale components in a random configuration.

FIG. 7 is a schematic diagram of a plurality of nano-scale components aligned in a chain.

FIG. 8 is a schematic diagram of a plurality of nano-scale components aligned in a specific configuration.

FIG. 9 shows an exemplary reaction mechanism for attaching artificial receptors to a carbon nanotube.

FIG. 10 is a schematic diagram of a plurality of nanotubes in a random configuration with a plurality of artificial receptors disposed thereon.

FIG. 11 is a schematic diagram of a plurality of nanotubes aligned into a lattice configuration.

FIG. 12 is a flow chart illustrating a process for creating a drug delivery device.

FIG. 13 is a schematic diagram showing a mixture of nanoparticles in the absence of the target component.

FIG. 14 is a schematic diagram showing an aggregation of nanoparticles.

FIG. 15 is a schematic diagram of a molecular tweezers with artificial receptors of the present invention disposed thereon.

FIG. 16 is a flow chart illustrating a process for creating a selective removal nanodevice.

FIG. 17 is a schematic diagram of an embodiment of a valve that employs the present artificial receptors.

FIG. 18 is a schematic diagram of a microstructure that includes the present artificial receptors.

FIG. 19 schematically illustrates identification of a lead artificial receptor from among candidate artificial receptors.

FIG. 20 schematically illustrates a false color fluorescence image of a labeled microarray according to an embodiment of the present invention.

FIG. 21 schematically illustrates a two dimensional plot of data obtained for candidate artificial receptors contacted with and/or binding phycoerythrin.

FIG. 22 schematically illustrates a three dimensional plot of data obtained for candidate artificial receptors contacted with and/or binding phycoerythrin.

FIG. 23 schematically illustrates a two dimensional plot of data obtained for candidate artificial receptors contacted with and/or binding a fluorescent derivative of ovalbumin.

FIG. 24 schematically illustrates a three dimensional plot of data obtained for candidate artificial receptors contacted with and/or binding a fluorescent derivative of ovalbumin.

FIG. 25 schematically illustrates a two dimensional plot of data obtained for candidate artificial receptors contacted with and/or binding a fluorescent derivative of bovine serum albumin.

FIG. 26 schematically illustrates a three dimensional plot of data obtained for candidate artificial receptors contacted with and/or binding a fluorescent derivative of bovine serum albumin.

FIG. 27 schematically illustrates a two dimensional plot of data obtained for candidate artificial receptors contacted with and/or binding an acetylated horseradish peroxidase.

FIG. 28 schematically illustrates a three dimensional plot of data obtained for candidate artificial receptors contacted with and/or binding an acetylated horseradish peroxidase.

FIG. 29 schematically illustrates a two dimensional plot of data obtained for candidate artificial receptors contacted with and/or binding a TCDD derivative of horseradish peroxidase.

FIG. 30 schematically illustrates a three dimensional plot of data obtained for candidate artificial receptors contacted with and/or binding a TCDD derivative of horseradish peroxidase.

FIG. 31 schematically illustrates a subset of the data illustrated in FIG. 22.

FIG. 32 schematically illustrates a subset of the data illustrated in FIG. 22.

FIG. 33 schematically illustrates a subset of the data illustrated in FIG. 22.

FIG. 34 schematically illustrates a correlation of binding data for phycoerythrin against logP for the building blocks making up the artificial receptor.

FIG. 35 schematically illustrates a correlation of binding data for phycoerythrin against logP for the building blocks making up the artificial receptor.

FIG. 36 schematically illustrates a two dimensional plot comparing data obtained for candidate artificial receptors contacted with and/or binding phycoerythrin to data obtained for candidate artificial receptors contacted with and/or binding a fluorescent derivative of bovine serum albumin.

FIGS. 37, 38, and 39 schematically illustrate subsets of data from FIGS. 22, 26, and 24, respectively, and demonstrate that the array of artificial receptors according to the present invention yields receptors distinguished between three analytes, phycoerythrin, bovine serum albumin, and ovalbumin.

FIG. 40 schematically illustrates a gray scale image of the fluorescence signal from a scan of a control plate which was prepared by washing off the building blocks with organic solvent before incubation with the test ligand.

FIG. 41 schematically illustrates a gray scale image of the fluorescence signal from a scan of an experimental plate which was incubated with 1.0 μg/ml Cholera Toxin B at 23° C.

FIG. 42 schematically illustrates a gray scale image of the fluorescence signal from a scan of an experimental plate which was incubated with 1.0 μg/ml Cholera Toxin B at 3° C.

FIG. 43 schematically illustrates a gray scale image of the fluorescence signal from a scan of an experimental plate which was incubated with 1.0 μg/ml Cholera Toxin B at 43° C.

FIGS. 44-46 schematically illustrate plots of the fluorescence signals obtained from the candidate artificial receptors illustrated in FIGS. 41-43.

FIG. 47 schematically illustrate plots of the fluorescence signals obtained from the combinations of building blocks employed in the present studies, when those building blocks are covalently linked to the support. Binding was conducted at 23° C.

FIG. 48 schematically illustrates the changes in fluorescence signal from individual combinations of covalently immobilized building blocks at 3° C., 23° C., or 43° C.

FIG. 49 schematically illustrates a graph of the changes in fluorescence signal from individual combinations of building blocks at 3° C., 23° C., or 43° C.

FIG. 50 schematically illustrates the data presented in FIG. 48 (lines marked A) and the data presented in FIG. 49 (lines marked B).

FIG. 51 schematically illustrates a graph of the fluorescence signal at 43° C. divided by the signal at 23° C. against the fluorescence signal obtained from binding at 23° C. for the artificial receptors with reversibly immobilized receptors.

FIG. 52 illustrates fluorescence signals produced by binding of cholera toxin to a microarray of the present candidate artificial receptors followed by washing with buffer in an experiment reported in Example 4.

FIG. 53 illustrates the fluorescence signals due to cholera toxin binding that were detected upon competition with GM1 OS (0.34 μM) in an experiment reported in Example 4.

FIG. 54 illustrates the ratio of the amount bound in the absence of GM1 OS to the amount bound in competition with GM1 OS(0.34 μM) in an experiment reported in Example 4.

FIG. 55 illustrates fluorescence signals produced by binding of cholera toxin to a microarray of the present candidate artificial receptors followed by washing with buffer in an experiment reported in Example 4 and for comparison with competition experiments using 5.1 μM GM1 OS.

FIG. 56 illustrates the fluorescence signals due to cholera toxin binding that were detected upon competition with GM1 OS (5.1 μM) in an experiment reported in Example 4.

FIG. 57 illustrates the ratio of the amount bound in the absence of GM1 OS to the amount bound in competition with GM1 OS(5.1 μM) in an experiment reported in Example 4.

FIG. 58 illustrates the fluorescence signals produced by binding of cholera toxin to the microarray of candidate artificial receptors alone and in competition with each of the three concentrations of GM1 in the experiment reported in Example 5.

FIG. 59 illustrates the ratio of the amount bound in the absence of GM1 OS to the amount bound upon competition with GM1 for the low concentration of GM1 employed in Example 5.

FIG. 60 illustrates the fluorescence signals produced by binding of cholera toxin to the microarray of candidate artificial receptors without pretreatment with GM1 in the experiment reported in Example 6.

FIGS. 61-63 illustrate the fluorescence signals produced by binding of cholera toxin to the microarray of candidate artificial receptors with pretreatment with GM1 (100 μg/ml, 10 μg/ml, and 1 μg/ml GM1, respectively) in the experiment reported in Example 6.

FIG. 64 illustrates the ratio of the amount bound in the presence of 1 μg/ml GM1 to the amount bound in the absence of GM1 in the experiment reported in Example 6.

### DETAILED DESCRIPTION OF THE INVENTION

Definitions

As used herein, the term “peptide” refers to a compound including two or more amino acid residues joined by amide bond(s).

As used herein, the terms “polypeptide” and “protein” refer to a peptide including more than about 20 amino acid residues connected by peptide linkages.

As used herein, the term “proteome” refers to the expression profile of the proteins of an organism, tissue, organ, or cell. The proteome can be specific to a particular status (e.g., development, health, etc.) of the organism, tissue, organ, or cell.

Reversibly immobilizing building blocks on a support couples the building blocks to the support through a mechanism that allows the building blocks to be uncoupled from the support without destroying or unacceptably degrading the building block or the support. That is, immobilization can be reversed without destroying or unacceptably degrading the building block or the support. In an embodiment, immobilization can be reversed with only negligible or ineffective levels of degradation of the building block or the support. Reversible immobilization can employ readily reversible covalent bonding or noncovalent interactions. Suitable noncovalent interactions include interactions between ions, hydrogen bonding, van der Waals interactions, and the like. Readily reversible covalent bonding refers to covalent bonds that can be formed and broken under conditions that do not destroy or unacceptably degrade the building block or the support.

A combination of building blocks immobilized on, for example, a support can be a candidate artificial receptor, a lead artificial receptor, or a working artificial receptor. That is, a heterogeneous building block spot on a slide or a plurality of building blocks coated on a tube or well can be a candidate artificial receptor, a lead artificial receptor, or a working artificial receptor. A candidate artificial receptor can become a lead artificial receptor, which can become a working artificial receptor.

As used herein the phrase “candidate artificial receptor” refers to an immobilized combination of building blocks that can be tested to determine whether or not a particular test ligand binds to that combination. In an embodiment, the combination includes one or more reversibly immobilized building blocks. In an embodiment, the candidate artificial receptor can be a heterogeneous building block spot on a slide or a plurality of building blocks coated on a tube or well.

As used herein the phrase “lead artificial receptor” refers to an immobilized combination of building blocks that binds a test ligand at a predetermined concentration of test ligand, for example at 10, 1, 0.1, or 0.01 μg/ml, or at 1, 0.1, or 0.01 ng/ml. In an embodiment, the combination includes one or more reversibly immobilized building blocks. In an embodiment, the lead artificial receptor can be a heterogeneous building block spot on a slide or a plurality of building blocks coated on a tube or well.

As used herein the phrase “working artificial receptor” refers to a combination of building blocks that binds a test ligand with a selectivity and/or sensitivity effective for categorizing or identifying the test ligand. That is, binding to that combination of building blocks describes the test ligand as belonging to a category of test ligands or as being a particular test ligand. A working artificial receptor can, for example, bind the ligand at a concentration of, for example, 100, 10, 1, 0.1, 0.01, or 0.001 ng/ml. In an embodiment, the combination includes one or more reversibly immobilized building blocks. In an embodiment, the working artificial receptor can be a heterogeneous building block spot on a slide or a plurality of building blocks coated on a tube, well, slide, or other support or on a scaffold.

As used herein the phrase “working artificial receptor complex” refers to a plurality of artificial receptors, each a combination of building blocks, that binds a test ligand with a pattern of selectivity and/or sensitivity effective for categorizing or identifying the test ligand. That is, binding to the several receptors of the complex describes the test ligand as belonging to a category of test ligands or as being a particular test ligand. The individual receptors in the complex can each bind the ligand at different concentrations or with different affinities. For example, the individual receptors in the complex each bind the ligand at concentrations of 100, 10, 1, 0.1, 0.01 or 0.001 ng/ml. In an embodiment, the combination includes one or more reversibly immobilized building blocks. In an embodiment, the working artificial receptor complex can be a plurality of heterogeneous building block spots or regions on a slide; a plurality of wells, each coated with a different combination of building blocks; or a plurality of tubes, each coated with a different combination of building blocks.

As used herein, the phrase “significant number of candidate artificial receptors” refers to sufficient candidate artificial receptors to provide an opportunity to find a working artificial receptor, working artificial receptor complex, or lead artificial receptor. As few as about 100 to about 200 candidate artificial receptors can be a significant number for finding working artificial receptor complexes suitable for distinguishing two proteins (e.g., cholera toxin and phycoerythrin). In other embodiments, a significant number of candidate artificial receptors can include about 1,000 candidate artificial receptors, about 10,000 candidate artificial receptors, about 100,000 candidate artificial receptors, or more.

Although not limiting to the present invention, it is believed that the significant number of candidate artificial receptors required to provide an opportunity to find a working artificial receptor may be larger than the significant number required to find a working artificial receptor complex. Although not limiting to the present invention, it is believed that the significant number of candidate artificial receptors required to provide an opportunity to find a lead artificial receptor may be larger than the significant number required to find a working artificial receptor. Although not limiting to the present invention, it is believed that the significant number of candidate artificial receptors required to provide an opportunity to find a working artificial receptor for a test ligand with few features may be more than for a test ligand with many features.

As used herein, the term “building block” refers to a molecular component of an artificial receptor including portions that can be envisioned as or that include one or more linkers, one or more frameworks, and one or more recognition elements. In an embodiment, the building block includes a linker, a framework, and one or more recognition elements. In an embodiment, the linker includes a moiety suitable for reversibly immobilizing the building block, for example, on a support, surface or lawn. The building block interacts with the ligand.

As used herein, the term “linker” refers to a portion of or functional group on a building block that can be employed to or that does (e.g., reversibly) couple the building block to a support, for example, through covalent link, ionic interaction, electrostatic interaction, or hydrophobic interaction.

As used herein, the term “framework” refers to a portion of a building block including the linker or to which the linker is coupled and to which one or more recognition elements are coupled.

As used herein, the term “recognition element” refers to a portion of a building block coupled to the framework but not covalently coupled to the support. Although not limiting to the present invention, the recognition element can provide or form one or more groups, surfaces, or spaces for interacting with the ligand.

As used herein, the phrase “plurality of building blocks” refers to two or more building blocks of different structure in a mixture, in a kit, or on a support or scaffold. Each building block has a particular structure, and use of building blocks in the plural, or of a plurality of building blocks, refers to more than one of these particular structures. Building blocks or plurality of building blocks does not refer to a plurality of molecules each having the same structure.

As used herein, the phrase “combination of building blocks” refers to a plurality of building blocks that together are in a spot, region, or a candidate, lead, or working artificial receptor. A combination of building blocks can be a subset of a set of building blocks. For example, a combination of building blocks can be one of the possible combinations of 2, 3, 4, 5, or 6 building blocks from a set of N (e.g., N=10-200) building blocks.

As used herein, the phrases “homogenous immobilized building block” and “homogenous immobilized building blocks” refer to a support or spot having immobilized on or within it only a single building block.

As used herein, the phrase “activated building block” refers to a building block activated to make it ready to form a covalent bond to a functional group, for example, on a support. A building block including a carboxyl group can be converted to a building block including an activated ester group, which is an activated building block. An activated building block including an activated ester group can react, for example, with an amine to form a covalent bond.

As used herein, the term “naïve” used with respect to one or more building blocks refers to a building block that has not previously been determined or known to bind to a test ligand of interest. For example, the recognition element(s) on a naïve building block has not previously been determined or known to bind to a test ligand of interest. A building block that is or includes a known ligand (e.g., GM1) for a particular protein (test ligand) of interest (e.g., cholera toxin) is not naïve with respect to that protein (test ligand).

As used herein, the term “immobilized” used with respect to building blocks coupled to a support refers to building blocks being stably oriented on the support so that they do not migrate on the support or release from the support. Building blocks can be immobilized by covalent coupling, by ionic interactions, by electrostatic interactions, such as ion pairing, or by hydrophobic interactions, such as van der Waals interactions.

As used herein a “region” of a support, tube, well, or surface refers to a contiguous portion of the support, tube, well, or surface. Building blocks coupled to a region can refer to building blocks in proximity to one another in that region.

As used herein, a “bulky” group on a molecule is larger than a moiety including 7 or 8 carbon atoms.

As used herein, a “small” group on a molecule is hydrogen, methyl, or another group smaller than a moiety including 4 carbon atoms.

As used herein, the term “lawn” refers to a layer, spot, or region of functional groups on a support, for example, at a density sufficient to place coupled building blocks in proximity to one another. The functional groups can include groups capable of forming covalent, ionic, electrostatic, or hydrophobic interactions with building blocks.

As used herein, the term “alkyl” refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₁₂ for straight chain, C₁-C₆ for branched chain). Likewise, cycloalkyls can have from 3-10 carbon atoms in their ring structure, for example, 5, 6 or 7 carbons in the ring structure.

The term “alkyl” as used herein refers to both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an ester, a formyl, or a ketone), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aryl alkyl, or an aromatic or heteroaromatic moiety. The moieties substituted on the hydrocarbon chain can themselves be substituted, if appropriate. For example, the substituents of a substituted alkyl can include substituted and unsubstituted forms of the groups listed above.

The phrase “aryl alkyl”, as used herein, refers to an alkyl group substituted with an aryl group (e.g., an aromatic or heteroaromatic group).

As used herein, the terms “alkenyl” and “alkynyl” refer to unsaturated aliphatic groups analogous in length and optional substitution to the alkyls groups described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents such as those described above for alkyl groups. The term “aryl” also includes polycyclic ring systems having two or more cyclic rings in which two or more carbons are common to two adjoining rings (the rings are “fused rings”) wherein at least one of the rings is aromatic, e.g., the other cyclic ring(s) can be cycloalkyls, cycloalkenyls, cycloalkynyls, aryls and/or heterocyclyls.

As used herein, the terms “heterocycle” or “heterocyclic group” refer to 3- to 12-membered ring structures, e.g., 3- to 7-membered rings, whose ring structures include one to four heteroatoms. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazohne, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents such as those described for alkyl groups.

As used herein, the term “heteroatom” as used herein means an atom of any element other than carbon or hydrogen, such as nitrogen, oxygen, sulfur and phosphorous.

Overview of the Artificial Receptor

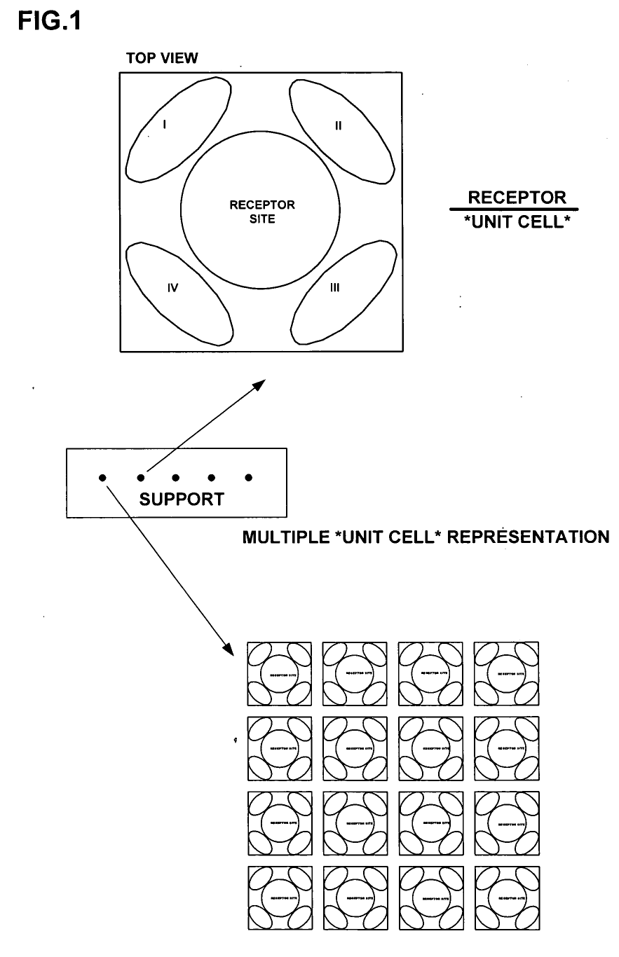

FIG. 1 schematically illustrates an embodiment employing 4 distinct building blocks in a spot on a microarray to make a ligand binding site. This Figure illustrates a group of 4 building blocks at the corners of a square forming a unit cell. A group of four building blocks can be envisioned as the vertices on any quadrilateral. FIG. 1 illustrates that spots or regions of building blocks can be envisioned as multiple unit cells, in this illustration square unit cells. Groups of unit cells of four building blocks in the shape of other quadrilaterals can also be formed on a support.

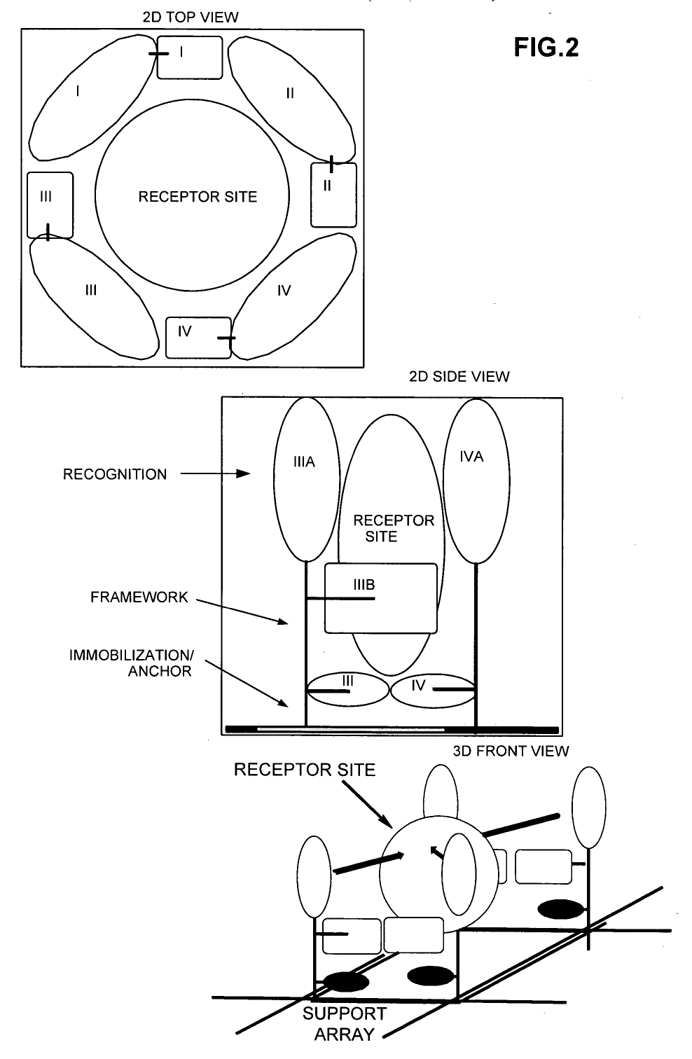

Each immobilized building block molecule can provide one or more “arms” extending from a “framework” and each can include groups that interact with a ligand or with portions of another immobilized building block. FIG. 2 illustrates that combinations of four building blocks, each including a framework with two arms (called “recognition elements”), provides a molecular configuration of building blocks that form a site for binding a ligand. Such a site formed by building blocks such as those exemplified below can bind a small molecule, such as a drug, metabolite, pollutant, or the like, and/or can bind a larger ligand such as a macromolecule or microbe.

The present artificial receptors can include building blocks reversibly immobilized on a support or surface. Reversing immobilization of the building blocks can allow movement of building blocks to a different location on the support or surface, or exchange of building blocks onto and off of the surface. For example, the combinations of building blocks can bind a ligand when reversibly coupled to or immobilized on the support. Reversing the coupling or immobilization of the building blocks provides opportunity for rearranging the building blocks, which can improve binding of the ligand. Further, the present invention can allow for adding additional or different building blocks, which can further improve binding of a ligand.

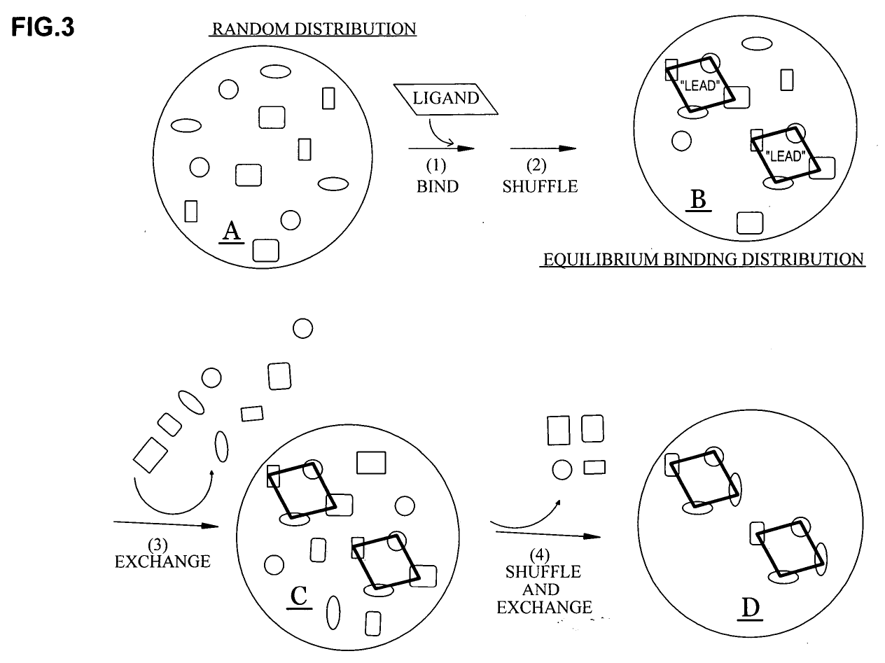

FIG. 3 schematically illustrates an embodiment employing an initial artificial receptor surface (A) with four different building blocks on the surface, which are represented by shaded shapes. This initial artificial receptor surface (A) undergoes (1) binding of a ligand to an artificial receptor and (2) shuffling the building blocks on the receptor surface to yield a lead artificial receptor (B). Shuffling refers to reversing the coupling or immobilization of the building blocks and allowing their rearrangement on the receptor surface. After forming a lead artificial receptor, additional building blocks can be (3) exchanged onto and/or off of the receptor surface (C). Exchanging refers to building blocks leaving the surface and entering a solution contacting the surface and/or building blocks leaving a solution contacting the surface and becoming part of the artificial receptor. The additional building blocks can be selected for structural diversity (e.g., randomly) or selected based on the structure of the building blocks in the lead artificial receptor to provide additional avenues for improving binding. The original and additional building blocks can then be (4) shuffled and exchanged to provide higher affinity artificial receptors on the surface (D).

General Methods Employing the Artificial Receptors

The present invention relates to nano- or microdevices or articles and methods of making and using them. The present devices, articles, or methods include or employ combinatorial artificial receptors. Such combinatorial artificial receptors can provide binding interactions for positioning or targeting the nano- or microdevice. For example, two devices or surfaces can be positioned or adhered to one another by using a combinatorial artificial receptor. In such an embodiment, each device or surface includes a combinatorial artificial receptor. The combinatorial artificial receptor on the first device or surface binds to the combinatorial artificial receptor on the second device or surface. Such a pair of receptors can be selected, for example, by screening one or more quantum dots having on their surface one or more building blocks against an array of candidate artificial receptors. Such screening methods are known.

By way of further example, a nano- or microdevice can include on a surface a combinatorial artificial receptor. This combinatorial artificial receptor can be selected for binding to a target surface or molecule. For example, the combinatorial artificial receptor can be selected for binding to a cell or tissue type. Such a nano- or microdevice can bind to that cell or tissue type, for example, when brought into contact with a biological sample or an organism.

Generally, working artificial receptors can be generated to be specific to a given test ligand or specific to a particular part of a given test ligand. As used herein, the phrase “test ligand” refers to a substance or molecule that can bind to or that is tested for binding to a candidate or working artificial receptor. Heterogeneous and immobilized combinations of building block molecules form the working artificial receptors. For example, combinations of 2, 3, 4, or 5 distinct building block molecules immobilized in proximity to one another on a support provide molecular structures that serve as candidate and working artificial receptors. The building blocks can be naïve to the test ligand.

Once a plurality of candidate artificial receptors are generated, they can be tested to determine which are specific or useful for a given ligand. For example, a plurality of candidate artificial receptors, such as an array of candidate artificial receptors may be screened with a labeled target ligand in order to find the working artificial receptors that have binding affinity for the target ligand. Binding of the labeled target ligand to an artificial receptor can be determined through a variety of methods known to those of skill in the art. These identified working artificial receptors can then be used on a nanotechnology based device or they can be further analyzed to isolate those with a desired binding affinity.

Artificial receptors according to the present invention can be used for various nanotechnology applications. By way of example, artificial receptors according to the present invention can be used for nanoassembly, nanotubes, nanowires, nanostructures, nano-scale drug delivery devices, nano-scale detectors, molecular tweezers, selective removal “garbage collecting” nanodevices, and other nanotechnology applications.

In an embodiment the invention includes a method of adhering components together. The method includes disposing a first artificial receptor on a first component, wherein the first artificial receptor includes a plurality of building blocks coupled to the first component, and wherein the first artificial receptor is known to having binding affinity for a second component. The method also includes allowing the artificial receptor to bind to the second component. In an embodiment, the first component and the second component includes nano-scale components. In an embodiment, the first component includes an item selected from the group consisting of a sheet, lattice, shell, wire, chain, ring, icosahedron, square pyramid, tetrahedron, staircase structure, sphere, tube, and helix. The method can also include disposing a second artificial receptor on the second component, wherein the second artificial receptor includes a plurality of building blocks coupled to the second component, and wherein the second artificial receptor is known to having binding affinity for the first artificial receptor.

In an embodiment, the invention is a device including a first component and a second component. The device can also include a first binding pair of artificial receptors including a first artificial receptor and a second artificial receptor. In an embodiment, the first and second artificial receptors each include a plurality of building blocks. The first artificial receptor can have binding affinity for the second artificial receptor. The first artificial receptor can be disposed on the first component and the second artificial receptor can be disposed on the second component. In an embodiment, the first component can be adhered to the second component via the first binding pair. The device can further include a third component and a second binding pair of artificial receptors including a third artificial receptor and a fourth artificial receptor. The third and the fourth artificial receptors can each include a plurality of building blocks, wherein the third artificial receptor is known to having binding affinity for the fourth artificial receptor; and wherein the third artificial receptor is disposed on the first component and the fourth artificial receptor is disposed on the third component. The first component can be adhered to the third component via the second binding pair of artificial receptors. In an embodiment, the device can include a sheet, lattice, shell, wire, chain, ring, icosahedron, square pyramid, tetrahedron, staircase structure, sphere, tube, or helix. In an embodiment, the first component and the second component can include nanotubes. The first artificial receptor can be covalently bonded to the first component. The second artificial receptor can be covalently bonded to the second component.

In an embodiment, the invention includes an agent delivery device having a capsule, and an active agent, wherein the active agent is disposed within the capsule. An artificial receptor can be disposed on the capsule, including a plurality of building blocks coupled to the capsule, wherein the artificial receptor is known to have binding affinity for a target ligand. In an embodiment, the agent delivery device can include a temperature-sensitive polymer and a metal nanoshell. The agent delivery device can also include a polyelectrolyte shell. The active agent can include thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandins, antidepressants, antipsychotic substances, neurotransmitters, anti-emetics, imaging agents, specific targeting agents, and cell response modifiers. In an embodiment, the target ligand can be a protein specific to a carcinoma cell. In an embodiment, the target ligand can be a molecule expressed by a microbe.

In an embodiment, the invention can include an agent delivery device having a nanotube, an active agent disposed on the nanotube, and a cap disposed on the nanotube having an open position and a closed position. The active agent can be prevented from vacating the nanotube when the cap is in the closed position. An artificial receptor can be disposed on the cap and can include a plurality of building blocks coupled to the cap, wherein the artificial receptor has a binding affinity for the nanotube that can be overcome by a release compound. In an embodiment, the cap is in the closed position when the artificial receptor is bound to the nanotube.

In an embodiment, the invention can include a detection device having a magnetic particle and an artificial receptor disposed on the magnetic particle. In an embodiment, the artificial receptor can include a plurality of building blocks coupled to the magnetic particle, wherein the artificial receptor is known to have binding affinity for a target ligand. The magnetic particle can include ferrite. The target ligand can include a drug of abuse, a disease marker, polynucleotide, a polypeptide, a microbe, a contaminant, or a small molecule.

In an embodiment, the invention can include a detection device having a quantum dot and an artificial receptor disposed on the quantum dot. The artificial receptor can include a plurality of building blocks coupled to the quantum dot. The artificial receptor can have binding affinity for a target ligand. The quantum dot can include silicon. The target ligand can include a drug of abuse, a disease marker, polynucleotide, a polypeptide, a microbe, a contaminant, or a small molecule.

In an embodiment, the invention can include a detection device having a plurality of first particles and a plurality of first artificial receptors disposed on the first particles. In an embodiment, the first artificial receptors can include a plurality of building blocks coupled to the first particles, and the first artificial receptors can have binding affinity for a first part of a target ligand. The detection device can also include a plurality of second particles and a plurality of second artificial receptors disposed on the second particles, the second artificial receptors including a plurality of building blocks coupled to the second particles, wherein the second artificial receptors are known to have binding affinity for a second part of a target ligand. In an embodiment, the first particles and the second particles aggregate in the present of the target ligand. The particles may include silicon. The particles may also include a quantum dot. The target ligand can include a drug of abuse, a disease marker, polynucleotide, a polypeptide, a microbe, a contaminant, or a small molecule.

In an embodiment, the invention can include a detection device having a cantilever and an artificial receptor disposed on the cantilever, the artificial receptor including a plurality of building blocks coupled to the cantilever, wherein the artificial receptor is known to have binding affinity for a target ligand. The detection device can include a plurality of cantilevers. The detection device can include a cantilever including silicon. The target ligand can include a drug of abuse, a disease marker, polynucleotide, a polypeptide, a microbe, a contaminant, or a small molecule.

In an embodiment, the invention can include a detection device having a substrate and an artificial receptor disposed on the substrate. The artificial receptor can have a plurality of building blocks coupled to the substrate, wherein the artificial receptor can have binding affinity for a target ligand. In an embodiment, the substrate has electrical properties that change when the target ligand is bound to the artificial receptor. The substrate can include a nanowire. The substrate can include a nanowire field effect transistor. The substrate can also include a nanotube. In an embodiment, the conductance of the substrate can change when the target ligand is bound to the artificial receptor. In an embodiment, the artificial receptor is covalently bound to the substrate. The target ligand can be a drug of abuse, a disease marker, polynucleotide, a polypeptide, a microbe, a contaminant, or a small molecule.

In an embodiment, the invention includes a device including a first nanotube tip and a second nanotube tip. In an embodiment, the first artificial receptor can be disposed on the first nanotube tip and the first artificial receptor including a plurality of building blocks can be coupled to the first nanotube tip, wherein the first artificial receptor is known to have binding affinity for a target ligand. In an embodiment, the second artificial receptor can be disposed on the second nanotube tip, the second artificial receptor can have a plurality of building blocks coupled to the second nanotube tip, wherein the second artificial receptor is known to have binding affinity for the target ligand. In an embodiment, the device can include a first electrode and a second electrode, wherein the first electrode is in electrical communication with the first nanotube tip and the second electrode is in electrical communication with the second nanotube tip. In an embodiment, the first artificial receptor and the second artificial receptor are the same.

In an embodiment, the invention can include a device for selective removal of a target component including a substrate and an artificial receptor disposed on the substrate, the artificial receptor including a plurality of building blocks coupled to the substrate, wherein the artificial receptor is known to have binding affinity for the target component. In an embodiment, the substrate enhances selective removal of the target component. The substrate can include a liposome. The substrate can be a magnetic bead. In an embodiment, the target component can be a lipophilic agent. The target component can also be a drug of abuse. In an embodiment, the target component can be a biological material. The target component can include a lipopolysaccharide.

Nanoassembly, Nanotubes, Nanowires, and Nanostructures

Artificial receptors according to the present invention can be used as a selective adhesive and with methods of nanoassembly. Artificial receptors can be created according to the invention that have binding affinity for a particular substrate, as described above. These artificial receptors can be disposed a substrate or a nanocomponent and can then be used to adhere the substrate or component to another substrate or component. By way of example, a first object, such as a conductive element, can be adhered to a second object, such as a nanosheet, by affixing an artificial receptor to the first object that has selective affinity for the second object. Such adhering can be employed to adhere two objects in a pattern. Alternatively, an artificial receptor that has selective affinity for the first object can be affixed to the second object. In this manner artificial receptors of the present invention can be used as a form of selective molecular adhesive or glue.

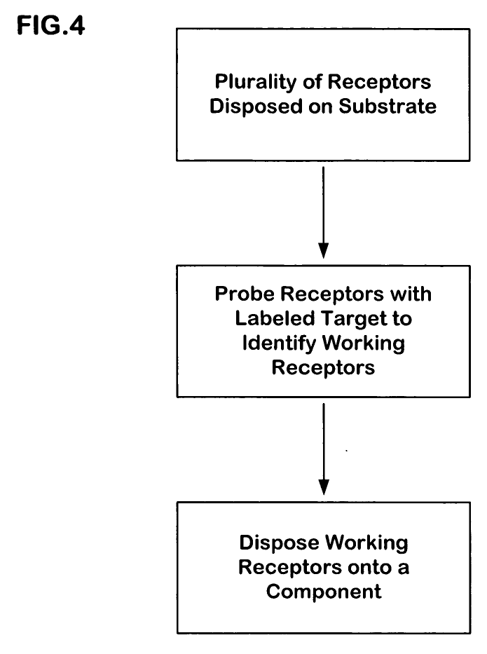

Referring to FIG. 4, this process is illustrated. First, a plurality of artificial receptors are disposed on a substrate, such as on an array. For example, a significant number of receptors can be disposed on a substrate. Then, a labeled target, such as a first component or a piece thereof, can be used to probe the artificial receptors on the array in order to find those that have binding affinity with the target molecule. Once a suitable receptor, or receptors, are identified, they can be disposed on a second component. Then the first component can be adhered to the second component based on binding between the working artificial receptor that is disposed on the second component and the first component.

Pairs of artificial receptors can also be created according to the invention that have complementary binding affinity. By way of example, a first artificial receptor can be produced which has selective binding affinity for a second artificial receptor, wherein the first and the second artificial receptor form an artificial receptor binding pair. In an embodiment, multiple pairs of artificial receptors that have distinct binding complementarity are created to specifically adhere components together. In this manner, assembly of a device on a nano-scale can be carried out in a specific manner because the individual artificial receptors will only have binding affinity for their complementary artificial receptor.

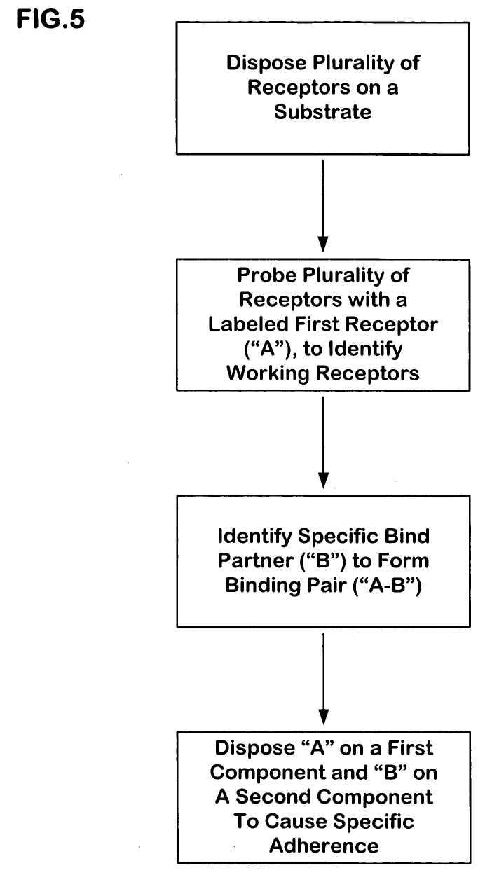

Referring to FIG. 5, the process of making pairs of artificial receptors is illustrated. First, a plurality of artificial receptors are disposed on a substrate, such as on an array. Then, a labeled target artificial receptor, such as a first receptor, can be used to probe the artificial receptors on the array in order to find those that have binding affinity with the target artificial receptor. These receptors with mutual binding affinity can be referred to as complementary working receptors.

Once a suitable receptor is identified to form a complementary binding pair with the target artificial receptor, the pair, or multiple pairs can be used to selectively adhere different components together. For example, a given complementary binding pair may consist of an “A” artificial receptor that specifically binds to a “B” artificial receptor. If the A receptor is disposed on a first component and the B receptor is disposed on a second component, then receptors A and B can be used to adhere the first component to the second component.

This type of selective adherence can be used for nano-scale assembly. By way of example, a nanotube or nanowire can have a plurality of “A” receptors on its surface. A nanosheet can have a plurality of “B” receptors on its surface. When the nanotube or wire is brought into contact with the nanosheet, the nanosheet can be adhered to the outside of the nanotube or wire thereby creating a multilayer nanowire. For example, the nanosheet may have insulating properties which aid in the functioning of the wire. In an embodiment, the nanosheet wraps around the nanotube or wire.

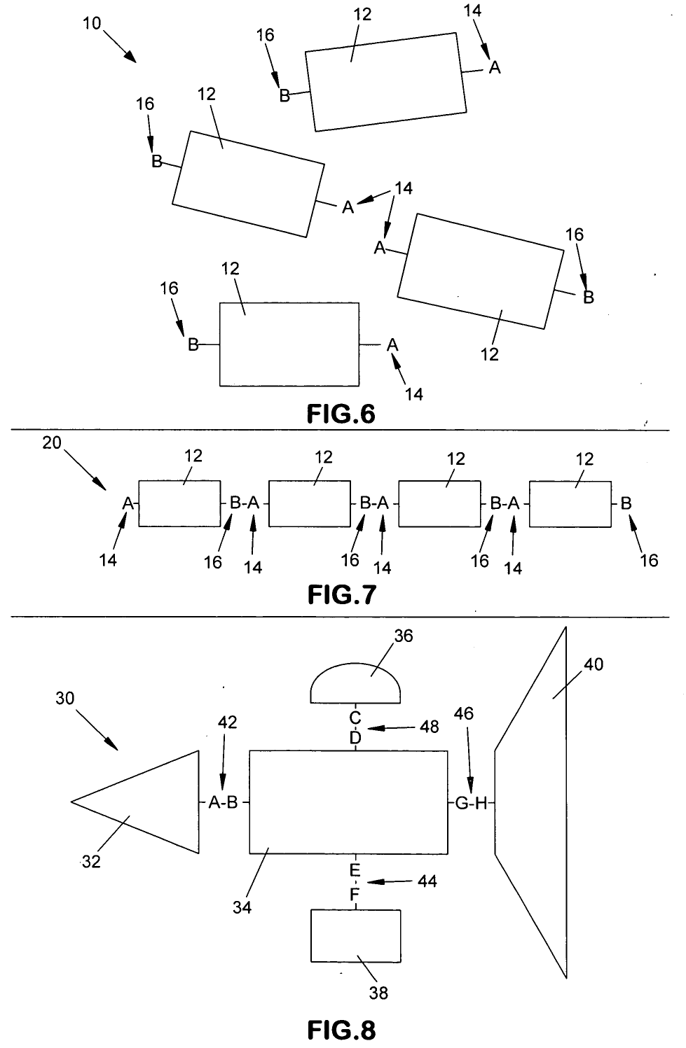

For example, referring now to FIGS. 6 and 7, the manner in which a plurality of A-B binding pairs are used to guide proper assembly of a nano-scale device is illustrated. As shown in FIG. 6, a plurality of components 12 begin in a random configuration. Each component has an “A” artificial receptor 14 and a “B” artificial receptor 16. As shown in FIG. 7, when these components have the opportunity to reorient themselves, for example when the components are in a solution, they will reorient themselves according to the A-B binding pairs that form. In this case, a chain 20 of components is formed.

Many different binding pairs are possible, and multiple different pairs can be used in an assembly system such that components of a nano-scale device are properly adhered together in the correct configuration. By way of example, this approach can be used to assemble sheets, shells, wires, chains, rings, icosahedra, square pyramids, tetrahedral, twisted and staircase structures, spheres, tubes, helices, and the like. By way of example, by disposing artificial receptors on a spherical particle at a relative azimuthal angle of one hundred eighty degrees, rings are possible. By changing the angle between the artificial receptors, the diameter of the ring can be controlled. These basic types of structures can in turn be assembled utilizing artificial receptors of the present invention into more complex shapes and devices.

In an embodiment, where binding pairs are disposed on different components to be assembled, self-assembly of the components is possible. This is, in part, because the instructions for assembly emerge from the nature of the forces acting between constituent components. Therefore, in an embodiment, once the members of artificial receptor binding pairs are appropriately disposed on components, the components will self-assemble into structures when they are given the chance to interact. Assembly or disassembly in this manner can be controlled by manipulating environmental variables such as temperature, solvent pH, salt concentration, and the like.

Referring now to FIG. 8, a plurality of different binding pairs can be used in order to guide proper assembly of a nano-device 30. A first component 32 is specifically adhered to a second component 34 via the A-B binding pair 42. A third component 36 is specifically adhered to the second component 34 via the C-D binding pair 48. A fourth component 38 is specifically adhered to the second component 34 via the E-F binding pair 44. Finally, a fifth component 40 is specifically adhered to the second component 34 via the G-H binding pair 46.

In an embodiment, artificial receptors of the present invention can be deposited on or adhered to the sidewalls of carbon nanotubes. The artificial receptors can be deposited such that they form highly regular patterns, such as with several nanometer gaps in between receptors. In this manner, the carbon nanotubes can be used to create structures such as lattice-type structures where carbon nanotubes are bound to one another at regular intervals with artificial receptors. In turn, lattice-type structures can be used for various purposes including as a molecular sieve. Lattice-type structures can also be used as structural components for more complex assemblies. In an embodiment, a lattice-type structure can be used to create a nano-stent or other tubular nano-lattice.

Artificial receptors can be covalently attached to the sidewalls of carbon nanotubes using the Bingel reaction. The reaction mechanism is believed to be nucleophilic addition of the deprotonated species of diethyl bromomalonate followed by an intramolecular substitution of the halogen in a [2+1] cycloaddition. The product of this reaction is a diester to which a plurality of building blocks, or one or more artificial receptors, can be attached. Building blocks or artificial receptors can be added to the diester in a variety of ways known to those of skill in the art. By way of example, various functional groups can be added through a transesterification reaction.

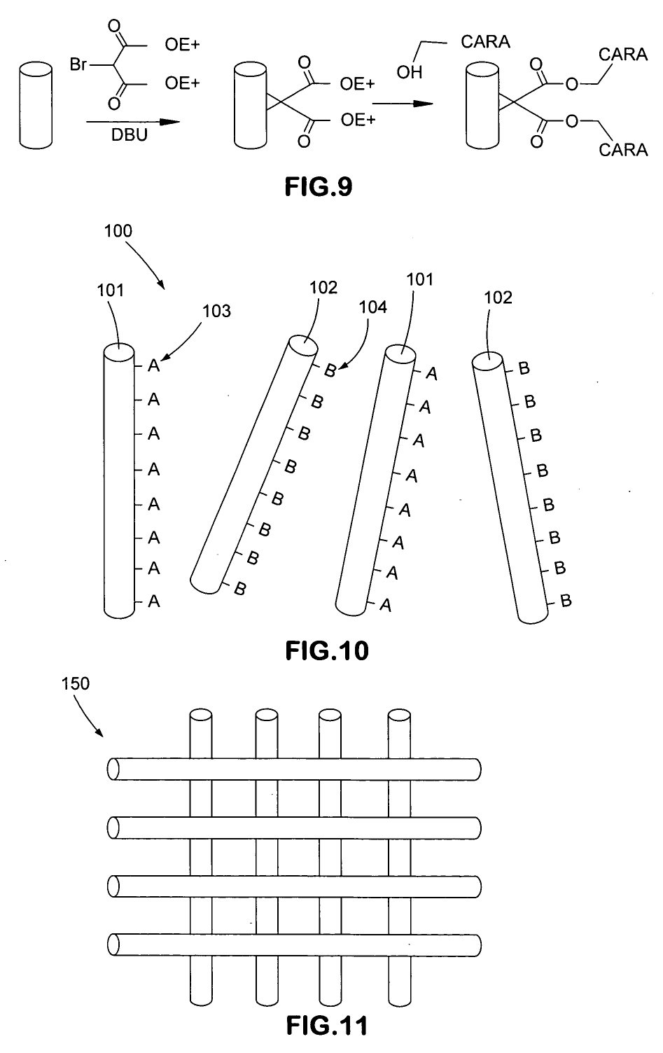

By way of example, single-walled carbon nanotubes can be added to a solvent, such as o-dichlorobenzene followed by sonication to disperse the nanotubes. By way of example, single-walled carbon nanotubes may be purchased from Carbon Nanotechnologies Inc., Houston, Tex. After sonication, an amount of 1,8-diazabicyclo[5.4.0]undec-7-ene (DBU) and diethyl bromomalonate can be added. At this point, a compound having an artificial receptor attached to an alcohol can be added. The artificial receptor then becomes covalently attached through a transesterification reaction catalyzed by an acid. The reaction is shown in FIG. 9. This reaction results in areas of functionalization alternating with unfunctionalized stretches.

Sonication can also be carried out during the reaction. It is believed that sonication of the reaction mixture accelerates the reaction and increases the degree of functionalization past the point of long-range periodicity. That is, the greater the amount of sonication, the more the reaction proceeds and some point the functionalized nanotubes will no longer exhibit regular intervals between functional groups (artificial receptors).

Once the functionalized nanotubes are created, they can be used to assemble structures such as lattices in a specific manner. For example, referring now to FIG. 10, one member of a binding pair of artificial receptors, A, is disposed on a first group of carbon nanotubes at regular distances along each nanotube. A second member of the binding pair, B, is disposed on a second group of carbon nanotubes at regular distances along each nanotube. When these two groups of carbon nanotubes are brought into close proximity to each other, they will form a lattice-type structure as shown in FIG. 11. This result can also be achieved by using an artificial receptor on one group of nanotubes and a target molecule that the nanotube specifically binds on the other group of nanotubes. More complicated structures can be achieved by using multiple different combinations of artificial receptor binding pairs.

Drug Delivery Devices

Artificial receptors according to the present invention can be used to form a drug delivery device. As described above, artificial receptors can be created according to the invention that have binding affinity for a given substrate (test ligand). In an embodiment, this substrate is a release compound and the working artificial receptor is disposed on a nano-scale drug delivery device. When the release compound, such as a protein characteristic of a carcinoma, bind to the working artificial receptor on the nano-scale drug delivery device, the device is triggered to release its payload of a drug. In this manner, therapeutic quantities of drugs can be released near the site they are needed, such as near a carcinoma cell, while minimizing drug side effects on non-targeted tissue.

In an embodiment, artificial receptors of the present invention are used with nanoparticle-based delivery systems. Such systems are known, for example, as in Dennis et al., U.S. Patent Application Publication U.S. 2004/0076681. By way of example, artificial receptors of the present invention may be disposed on a nanotube with one open end. The artificial receptors can specifically bind a target molecule on a cap structure. In this manner, the binding of the artificial receptor with the cap structure seals in the contents of the nanotube delivery vehicle until something disrupts the binding of the artificial receptor with the target molecule. In an embodiment, the disruptor may be a molecule that is characteristic of the proposed site of action, such as an aberrantly expressed protein from carcinoma cells.

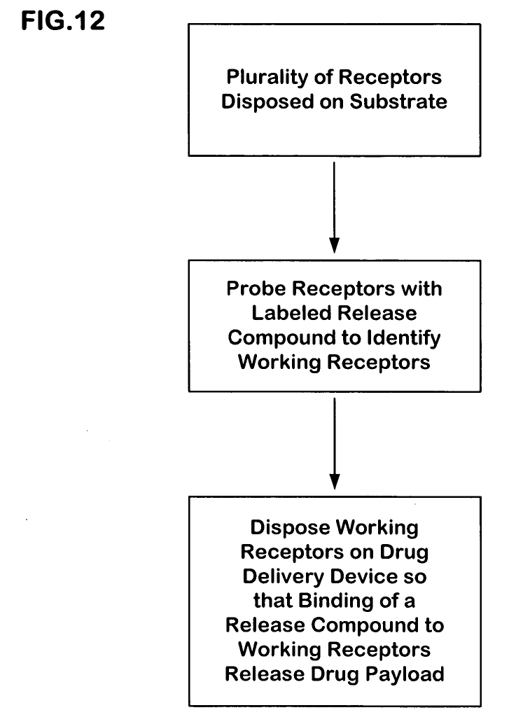

Referring to FIG. 12, a process for creating a drug delivery device is illustrated. First, a plurality of artificial receptors are disposed on a substrate, such as on an array. Then, a labeled release compound, such as a protein characteristic of a carcinoma, can be used to probe the artificial receptors on the array in order to find working receptors that have binding affinity with the release compound. Once a suitable working receptor, or working receptors, are identified, they can be disposed on a drug delivery device in a manner so as to allow release of a drug payload when a release compound binds to the receptor.

In an embodiment, the artificial receptors of the present invention may be used in combination with temperature-sensitive polymer/nanoshell composites for photothermally modulated drug delivery devices. For example, temperature-sensitive polymer/nanoshell composites are disclosed in West et al., U.S. Pat. No. 6,428,811 and metal nanoshells are disclosed in Oldenburg et al., U.S. Pat. No. 6,344,272. Metal nanoshells are nanoparticulate materials that can be tailored to absorb any desired wavelength and produce heat. For example, metal nanoshells can be created that absorb light in the near-infrared range and produce heat. Such nanoshells can be combined with a temperature-sensitive material to provide an implantable or injectable material for modulated drug delivery via external exposure to near-IR light. Artificial receptors of the present invention that are specific for a disease marker molecule can be disposed on the photothermally modulated drug delivery device in order to enhance localization of the drug delivery devices in vivo. Artificial receptors that are specific for a disease marker can be generated and identified as described above.

In an embodiment, artificial receptors of the present invention can be conjugated with nanoparticles that can mediate delivery of a compound, drug, or active agent. By way of example, particles that can comprise drug release capsules on the nano- or micro-scale are described in U.S. Pat. No. 6,699,501 (Neu et al. Artificial receptors of the present invention that are specific for a target ligand that is characteristic of a certain tissue type or microorganism can be conjugated to these drug release capsules in order to mediate tissue or site specific release of a compound, drug, or active agent. Working artificial receptors that are specific for a given target ligand can be generated as described above. These working artificial receptors can then be conjugated to the drug release capsules described in U.S. Pat. No. 6,699,501 by means known to those of skill in the art.

The compound or drug that is selectively delivered can include thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandins, antidepressants, antipsychotic substances, neurotransmitters, anti-emetics, imaging agents, specific targeting agents, and cell response modifiers.

More specifically, in embodiments the compound or drug can include heparin, covalent heparin, synthetic heparin salts, or another thrombin inhibitor; hirudin, hirulog, argatroban, D-phenylalanyl-L-poly-L-arginyl chloromethyl ketone, or another antithrombogenic agent; urokinase, streptokinase, a tissue plasminogen activator, or another thrombolytic agent; a fibrinolytic agent; a vasospasm inhibitor; a calcium channel blocker, a nitrate, nitric oxide, a nitric oxide promoter, nitric oxide donors, dipyridamole, or another vasodilator; HYTRIN® or other antihypertensive agents; a glycoprotein IIb/IIIa inhibitor (abciximab) or another inhibitor of surface glycoprotein receptors; aspirin, ticlopidine, clopidogrel or another antiplatelet agent; colchicine or another antimitotic, or another microtubule inhibitor; dimethyl sulfoxide (DMSO), a retinoid, or another antisecretory agent; cytochalasin or another actin inhibitor; cell cycle inhibitors; remodeling inhibitors; deoxyribonucleic acid, an antisense nucleotide, or another agent for molecular genetic intervention; methotrexate, or another antimetabolite or antiproliferative agent; tamoxifen citrate, TAXOL®, paclitaxel, or the derivatives thereof, rapamycin, vinblastine, vincristine, vinorelbine, etoposide, tenopiside, dactinomycin (actinomycin D), daunorubicin, doxorubicin, idarubicin, anthracyclines, mitoxantrone, bleomycin, plicamycin (mithramycin), mitomycin, mechlorethamine, cyclophosphamide and its analogs, chlorambucil, ethylenimines, methylmelamines, alkyl sulfonates (e.g., busulfan), nitrosoureas (carmustine, etc.), streptozocin, methotrexate (used with many indications), fluorouracil, floxuridine, cytarabine, mercaptopurine, thioguanine, pentostatin, 2-chlorodeoxyadenosine, cisplatin, carboplatin, procarbazine, hydroxyurea, or other anti-cancer chemotherapeutic agents; cyclosporin, tacrolimus (FK-506), azathioprine, mycophenolate mofetil, mTOR inhibitors, or another immunosuppressive agent; cortisol, cortisone, dexamethasone, dexamethasone sodium phosphate, dexamethasone acetate, dexamethasone derivatives, betamethasone, fludrocortisone, prednisone, prednisolone, 6U-methylprednisolone, triancinolone (e.g., triamcinolone acetonide), or another steroidal agent; trapidil (a PDGF antagonist), angiopeptin (a growth hormone antagonist), angiogenin, a growth factor (such as vascular endothelial growth factor (VEGF)), or an anti-growth factor antibody, or another growth factor antagonist or agonist; dopamine, bromocriptine mesylate, pergolide mesylate, or another dopamine agonist; ⁶⁰Co (5.3 year half life), ¹⁹²Ir (73.8 days), ³²P (14.3 days), ¹¹¹In (68 hours), ⁹⁰Y (64 hours), ⁹⁹Tc (6 hours), or another radiotherapeutic agent; iodine-containing compounds, barium-containing compounds, gold, tantalum, platinum, tungsten or another heavy metal functioning as a radiopaque agent; a peptide, a protein, an extracellular matrix component, a cellular component or another biologic agent; captopril, enalapril or another angiotensin converting enzyme (ACE) inhibitor; angiotensin receptor blockers; enzyme inhibitors (including growth factor signal transduction kinase inhibitors); ascorbic acid, alpha tocopherol, superoxide dismutase, deferoxamine, a 21-aminosteroid (lasaroid) or another free radical scavenger, iron chelator or antioxidant; a ¹⁴C-, ³H-, ¹³¹I-, ³²P or ³⁶S-radiolabelled form or other radiolabelled form of any of the foregoing; estrogen or another sex hormone; AZT or other antipolymerases; acyclovir, famciclovir, rimantadine hydrochloride, ganciclovir sodium, Norvir, Crixivan, or other antiviral agents; 5-aminolevulinic acid, meta-tetrahydroxyphenylchlorin, hexadecafluorozinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine beta-hydroxylase conjugated to saporin, or other antibody targeted therapy agents; gene therapy agents; enalapril and other prodrugs; PROSCAR®, HYTRIN® or other agents for treating benign prostatic hyperplasia (BHP); mitotane, aminoglutethimide, breveldin, acetaminophen, etodalac, tolmetin, ketorolac, ibuprofen and derivatives, mefenamic acid, meclofenamic acid, piroxicam, tenoxicam, phenylbutazone, oxyphenbutazone, nabumetone, auranofin, aurothioglucose, gold sodium thiomalate, a mixture of any of these, or derivatives of any of these. A comprehensive listing of compounds or drugs can be found in The Merck Index, Thirteenth Edition, Merck & Co. (2001).

Antibiotics are substances which inhibit the growth of or kill microorganisms. Antibiotics can be produced synthetically or by microorganisms. Examples of antibiotics include penicillin, tetracycline, chloramphenicol, minocycline, doxycycline, vancomycin, bacitracin, kanamycin, neomycin, gentamycin, erythromycin and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., either by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Antiviral agents are substances capable of destroying or suppressing the replication of viruses. Examples of anti-viral agents include α-methyl-1-adamantanemethylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCL, tacrine, 1-hydroxy maleate, iodotubercidin, p-bromotetramisole, 10-(α-diethylaminopropionyl)-phenothiazine hydrochloride, calmidazolium chloride, hemicholinium-3,3,5-dinitrocatecho-1, diacylglycerol kinase inhibitor I, diacylglycerol kinase inhibitor II, 3-phenylpropargylaminie, N-monomethyl-L-arginine acetate, carbidopa, 3-hydroxybenzylhydrazine HCl, hydralazine HCl, clorgyline HCl, deprenyl HCl L(−), deprenyl HCl D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-di-phenylvalerate hydrochloride, 3-isobutyl-1-methylxanthne, papaverine HCl, indomethacind, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate R(+), p-aminoglutethimide tartrate S(−), 3-iodotyrosine, alpha-methyltyrosine L(−), alpha-methyltyrosine D(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide.

Local anesthetics are substances that have an anesthetic effect in a localized region. Examples of such anesthetics include procaine, lidocaine, tetracaine and dibucaine.

Imaging agents are agents capable of imaging a desired site, e.g., tumor, in vivo. Examples of imaging agents include substances having a label that is detectable in vivo, e.g., antibodies attached to fluorescent labels. The term antibody includes whole antibodies or fragments thereof.

Cell response modifiers are chemotactic factors such as platelet-derived growth factor (PDGF). Other chemotactic factors include neutrophil-activating protein, monocyte chemoattractant protein, macrophage-inflammatory protein, SIS (small inducible secreted), platelet factor, platelet basic protein, melanoma growth stimulating activity, epidermal growth factor, transforming growth factor alpha, fibroblast growth factor, platelet-derived endothelial cell growth factor, insulin-like growth factor, nerve growth factor, bone growth/cartilage-inducing factor (alpha and beta), and matrix metalloproteinase inhibitors. Other cell response modifiers are the interleukins, interleukin receptors, interleukin inhibitors, interferons, including alpha, beta, and gamma; hematopoietic factors, including erythropoietin, granulocyte colony stimulating factor, macrophage colony stimulating factor and granulocyte-macrophage colony stimulating factor; tumor necrosis factors, including alpha and beta; transforming growth factors (beta), including beta-1, beta-2, beta-3, inhibin, activin, and DNA that encodes for the production of any of these proteins, antisense molecules, androgenic receptor blockers and statin agents.

The release compound can be a molecule that is characteristic of the proposed site of action, such as an protein from carcinoma cells or a surface protein of a microorganism.

Nano-Scale Detectors

Artificial receptors of the present invention may be used to form nanodevices that are useful for detection of a desired molecule or group of molecules. By way of example, artificial receptors may be used for purposes of diagnosis of disease, for detection of drugs of abuse, for identification of a sequence of a polynucleotide or protein, etc.

In an embodiment, an artificial receptor of the present invention is conjugated to a nano-scale magnetic particle. The artificial receptor can be made to be specific to any desired target molecule as described above. When the artificial receptor/magnetic particle conjugate is bound to a target, it can be induced to create a detectable magnetic field. In contrast, unbound particles do not create a detectable magnetic field. In this manner, a specific target can be quickly and easily tested for.

In an embodiment, the invention includes a method for making an artificial receptor/magnetic particle conjugate. First a working artificial receptor that has specific binding affinity for a particular test ligand can be generated as described above. Next, a magnetic nano-particle is obtained. By way of example, the magnetic nano-particle may comprise ferrite or the like. The working artificial receptor is then disposed on the magnetic nano-particle through means known to those of skill in the art.

Quantum dots are semiconductor nanocrystals which, after being energized, will emit light in a wavelength that can be predetermined by controlling the size of the nanocrystal. In this manner, the quantum dots can be used a photo-marker in various assays. Different quantum dots can also be encapsulated together into a nano-aggregate that will have a characteristic combination of light wave-lengths emitted. In this manner, the aggregations of different quantum dots can serve as a unique marker analogous to barcode markings. In an embodiment, quantum dots, or aggregations of quantum dots, are conjugated to an artificial receptor of the invention to create a nano-scale identification device.

In an embodiment, the invention includes a method for making a quantum dot/artificial receptor conjugate. First a working artificial receptor that has specific binding affinity for a particular test ligand can be generated as described above. Next, a quantum dot is obtained. By way of example, the quantum dot may comprise silicon, germanium, cadmium, selenium, or other components. Quantum dots may be formed, for example, as described in U.S. Pat. No. 6,774,014 (Lee et al.) or U.S. Pat. No. 6,596,555 (Bensahel et al.). Quantum dots are also commercially available from, for example, Evident Technologies, Troy, N.Y. The working artificial receptor is then disposed on the quantum dot through means known to those of skill in the art.

In an embodiment, artificial receptors of the present invention can be used in particle-aggregation based assays materials and methods. For example, for a given target component to be detected, a receptor that is specific for a first part of the target component can be attached to a first set of beads, or particles. Then a different receptor that is specific for a second part of the target component can be attached to a second set of beads, or particles. When both sets of beads or particles are then added to a test sample, if the target component is present, it will cause an aggregation of the particles, which will be observable. In this manner, detection of an aggregation will be a positive indicator for the presence of the target component.

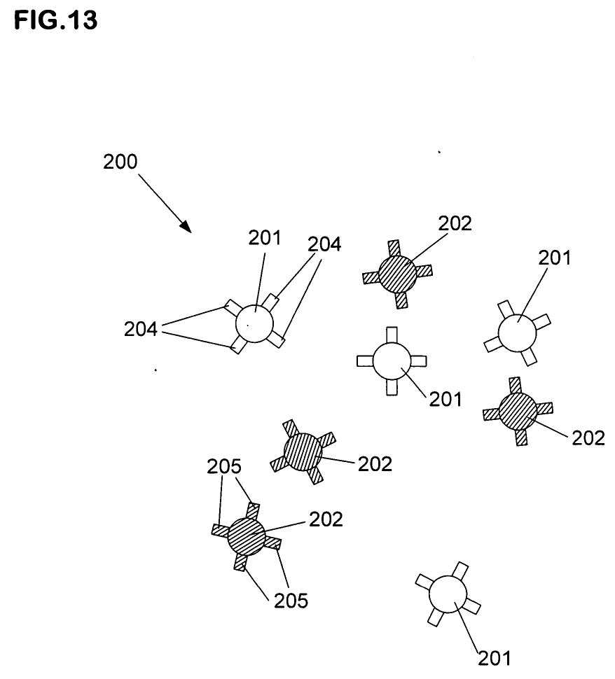

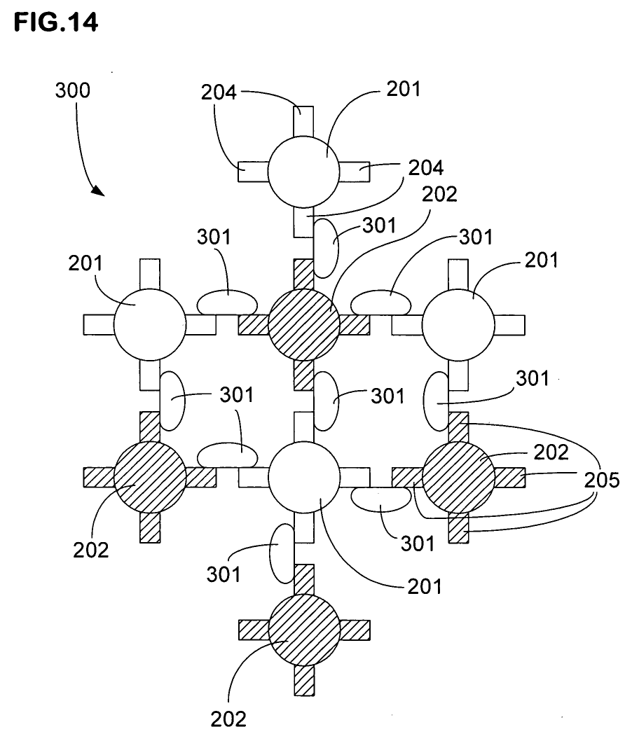

Referring to FIG. 13, a mixture of nanoparticles 200 is shown in the absence of the target component. A first set of nanoparticles 201 with first working receptors 204 disposed thereon is randomly oriented among a second set of nanoparticles 202 with second working receptors 205 disposed thereon. Referring to FIG. 14, an aggregation 300 of nanoparticles is shown. The first set of nanoparticles 201 is now aggregated with the second set of nanoparticles 202 based on target components 301 that form a link between first working receptors 204 and second working receptors 205. This aggregation 300 of nanoparticles indicates the presence of the target components 301.

In an embodiment, the invention includes a method for making particle aggregation assay materials. A first working artificial receptor that has specific binding affinity for a test ligand that corresponds to a first part of a target component can be generated as described above. A second working artificial receptor that has specific binding affinity for a different test ligand that corresponds to a second part of a target component can be generated as described above. Next, copies of the first working artificial receptor are disposed on a plurality of substrates, such as a plurality of particles, for example nano-scale particles. Then, copies of the second working artificial receptor are disposed on a second plurality of substrates, such as a plurality of particles, for example nano-scale particles. When the first and second pluralities of substrates are combined in the presence of an unknown sample, they will aggregate if the target component is present. This is because the target component will serve as a link between first and second pluralities of particles.

In an embodiment, artificial receptors of the present invention can be used in a cantilever-based detection device. A cantilever-based detection device is one in which one or more beams of silicon form cantilevers that can flex in response to forces. On the nanoscale, binding of a component to the cantilever can cause a movement of the cantilever based on surface stress. This movement is detectable. For example, the movement or bending of the cantilever is detectable by a beam deflection technique. However, one of skill in the art will appreciate that other techniques are possible for detecting bending of the cantilever. For example, binding of a component to a cantilever can also be detected through such techniques as measuring binding-induced resonance frequency shifts.

When artificial receptors of the present invention are disposed on the cantilever arms, the binding of target compounds to the artificial receptors can be detected. In an embodiment, artificial receptors of the present invention are deposited on one or more cantilevers to form a nanoscale cantilever-based detection device. Any type of compound that specifically binds with an artificial receptor can be detected in this manner. By way of example, such nanoscale cantilever-based devices could detect biological materials, drugs, biohazardous agents, etc.

In an embodiment, a plurality of cantilevers are attached to a detection device with a plurality of artificial receptors that are specific for different target compounds to form a cantilever array. The cantilever array can detect the presence of a plurality of specific target compounds simultaneously.

In an embodiment, the cantilever arm can be made of silicon. However, one of skill in the art will appreciate that other materials may also be used. Nanocantilevers can be fabricated using many different techniques including the use of focused ion beam techniques.

In an embodiment, the invention includes a method for making a detection device comprising an artificial receptor disposed on a nanocantilever. First a working artificial receptor that has specific binding affinity for a particular test ligand can be generated as described above. Then the working artificial receptor is disposed on a cantilever arm through means known to those of skill in the art. If a detection device that can detect multiple different components simultaneously is desired, then multiple cantilever arms are used with different working receptors disposed on each cantilever arm.

In an embodiment, nanowire field-effect transistors can be converted into sensors by modifying their surfaces with artificial receptors. It is believed that the interaction of a charged analyte with an artificial receptor of the present invention that is disposed on a conductive sensor element carries with it a field effect that modulates the electrical properties (such as conductance) of the sensor element. In an embodiment, the conductive sensor element is a nanowire. The small size of “nanostructures” allows for substantially increased sensitivity, since the field effect of a bound analyte affects a greater portion of the sensor element than the larger sensors that had been previously described. Specifically, the field effect of a bound analyte modulates the conductance across a greater percentage of the cross section of the nanowire or nanotube, and thus more substantially affects its measurable conductance. Nanosensors are described in Pontis et al., U.S. Patent Application 2004/0136866. Therefore, in an embodiment of the invention, binding of a target molecule to the artificial receptor can be detected by monitoring the electrical properties of the nanowire field-effect transistor.

In an embodiment, the invention includes a method for making a detection device comprising an artificial receptor disposed on a nanowire field-effect transistor. First a working artificial receptor that has specific binding affinity for a particular test ligand can be generated as described above. Next, a nanowire is obtained. Nanowires may be formed, for example, as described in U.S. Pat. No. 6,720,240 (Gole et al.). Nanowires are also commercially available from, for example, Nano Lab, Newton, Mass. The working artificial receptor is then disposed on the nanowire through means known to those of skill in the art.

Similarly, in an embodiment, artificial receptors of the present invention can be bound to the surface of a carbon nanotube in order to create a sensor. It is believed that the conductance of a carbon nanotube with one or more receptors bound to its surface will change when the receptors bind to a target molecule that they are specific for. Therefore, a sensor can be created that is specific for any desired component by monitoring the conductance through the carbon nanotube.

In an embodiment, the invention includes a method for making a detection device comprising an artificial receptor disposed on the surface of a nanotube. First a working artificial receptor that has specific binding affinity for a particular test ligand can be generated as described above. Next, a nanotube is obtained. Nanotubes may be formed, for example, as described in U.S. Pat. No. 6,451,175 (Lal et al.). Nanotubes are also commercially available from, for example, Nano Lab, Newton, Mass. The working artificial receptor is then disposed on the nanotube through means known to those of skill in the art. By way of example, a working artificial receptor may be disposed on a nanotube through the Bingel reaction described above.

Transparent conductive films may be formed from nanotubes. In an embodiment, the invention includes a detection device having an artificial receptor disposed on a transparent conductive film. It is believed that the transmittance of the film will vary when a target ligand binds to an artificial receptor that has been disposed on the film. Therefore, the presence of the target ligand can be determined based on the transmittance of the film.

Molecular Tweezers

Artificial receptors according to the present invention can be used as molecular tweezers. As described above, artificial receptors can be created according to the invention that have binding affinity for a target substrate. Nanotweezers describe a device having at least two nanotube tips that are each in contact with independent electrodes. When a voltage is applied between the electrodes, the spacing between the ends of the nanotube tips changes so that the nanotweezers can be used to manipulate objects. When artificial receptors of the present invention are disposed on the nanotube tips, the molecular tweezers can more effectively be used to grasp an object comprising a specific target substrate.

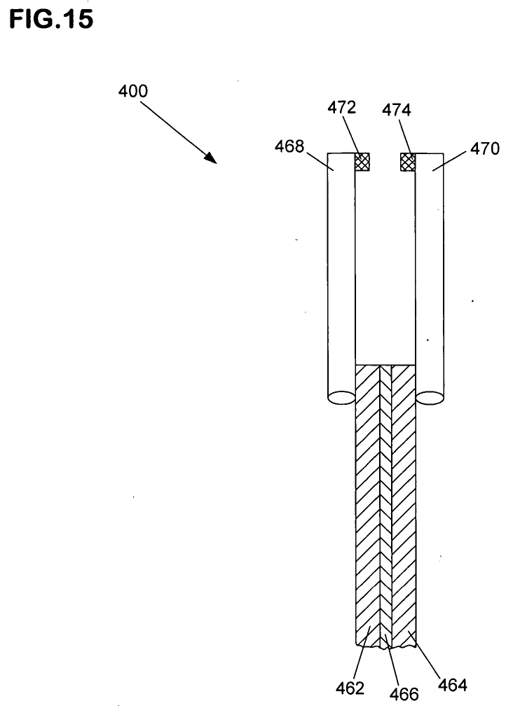

Referring to FIG. 15, a schematic diagram of a molecular tweezers 400 with artificial receptors of the present invention disposed thereon is shown. A first electrode 462 is separated from a second electrode 464 by an insulator 466. A first nanotube 468 is attached to the first electrode 462 and a second nanotube 470 is attached to the second electrode 464. A first artificial receptor 472 is disposed on the first nanotube 468 and a second artificial receptor 474 is disposed on the second nanotube 470. The first artificial receptor 472 and the second artificial receptor 474 may be the same or may be different. In embodiments where the first artificial receptor 472 and the second artificial receptor 474 are different, they can be used to grasp a molecule or object in a particular orientation. For example, where the first artificial receptor 472 is specific for a first side of a molecule or object and the second artificial receptor 474 is specific for a second side of a molecule or object, the tweezers will be able to pick of the molecule or object in a particular orientation.

In an embodiment, the invention includes a method for making a device comprising an artificial receptor disposed on the surface of a nanotube from a molecular tweezers. Methods for creating a molecular tweezers without artificial receptors are described in Lieber et al., U.S. Pat. No. 6,743,408. Then a working artificial receptor that has specific binding affinity for a particular test ligand can be generated as described above. Next, the working artificial receptor with the desired specific binding affinity is disposed on one of the carbon nanotubes. By way of example, a working artificial receptor may be disposed on a nanotube through the Bingel reaction described above. Then if, desired, a second working artificial receptor having either the same or different binding specificity as the first working artificial receptor can be disposed on the other carbon nanotube.

Selective Removal “Garbage Collecting” Nanodevices

Artificial receptors according to the present invention can be used to form a selective removal device. As described above, artificial receptors can be created according to the invention that have binding affinity for a target substrate. When the target substrate is a component that is to be removed (“garbage”), the artificial receptors can be bound to something that facilitates removal of this garbage. For example, an artificial receptor that specifically binds lipopolysaccharide (LPS) can be conjugated to a magnetic bead. Then a sample can be cleaned of whatever LPS it contains by adding an amount of these artificial receptor conjugates to the sample and then, after allowing binding to occur, a magnetic force can be applied selectively removing the LPS (“garbage”) from the sample.

In an embodiment, artificial receptors of the present invention that are specific for a given type of “garbage” to be removed may be mounted or embedded on or in the surface of a liposome in order to enhance the functioning of the liposome to remove the specific type of “garbage” desired, such as lipophilic compounds for which the artificial receptor has binding affinity.

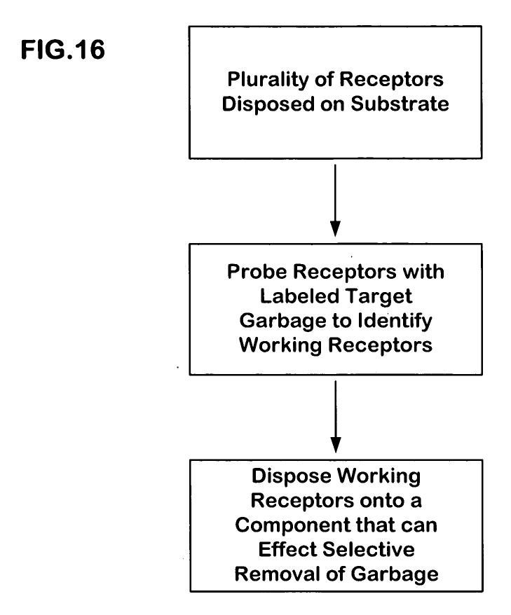

Referring to FIG. 16, a process for creating a selective removal nanodevice is illustrated. First, a plurality of artificial receptors are disposed on a substrate, such as on an array. For example, a significant number of receptors can be disposed on a substrate. Then, a piece of labeled target garbage can be used to probe the artificial receptors on the array in order to find working receptors that have binding affinity with the target garbage. Once a suitable receptor, or receptors, are identified, they can be conjugated to a component that will facilitate removal of the garbage.

The garbage can be any type of material one desires to selectively remove. For example, the garbage can be biological materials, left-over components after a nano-scale assembly process, malformed or aberrant nano-scale components, waste products, etc.

In some embodiments, the “garbage” to be selectively removed may comprise a drug of abuse or metabolite thereof. In an embodiment, the “garbage” may be an overdosage of a therapeutic agent. For example, bupivacaine, a potent local anesthetic, when administered to rats in a sufficient amount can cause their hearts to stop beating. In an embodiment, a garbage collector device that removes bupivacaine can be constructed by attaching an artificial receptor that is specific for bupivacaine to a moiety that will enhance clearance of bupivacaine.

In an embodiment, artificial receptors of the present invention are specific for a surface of a nanodevice that may be present in an organism. For example, where nanodevices are administered to an organism for a therapeutic purpose, it may be desired to remove them at a later point in time. By using artificial receptors of the present invention that are specific for a given surface of a nanodevice to be removed, clearance can be enhanced where the artificial receptor is in turn conjugated to a molecule that allows for selective removal.

Other Nanotechnology Applications:

Atomic force microscopes (AFMs) typically operate by scanning a fine ceramic or semiconductor tip over a test surface much the same way as a phonograph needle scans a record. The tip is positioned at the end of a cantilever beam shaped much like a diving board. As the tip is repelled by or attracted to the surface, the cantilever beam deflects. The magnitude of the deflection is captured by a laser that reflects at an oblique angle from the very end of the cantilever. A plot of the laser deflection versus tip position on the sample surface provides the resolution of the hills and valleys that constitute the topography of the test surface. The AFM can work with the tip touching the sample (contact mode), or the tip can tap across the surface (tapping mode) much like the cane of a blind person.

In an embodiment, the artificial receptors of the present invention can be disposed on the tip of an AFM such that any particular target molecule will then bind to the artificial receptor and serve as the end of the AFM tip. This can allow flexibility in terms of what type of material to dispose on the tip. The target molecule could be a ceramic, a semiconductor, or any other material that functions depending on the type of target surface to be scanned.

In an embodiment, artificial receptors of the present invention can be used in conjunction with fluidic systems on either the nano- or micro-scale. By way of example, U.S. Pat. No. 6,767,194 (Jeon et al.) describes valves and pumps for microfluidic systems and methods for making microfluidic systems. The artificial receptors of the present invention can be disposed on the microfluidic systems described by Jeon et al.

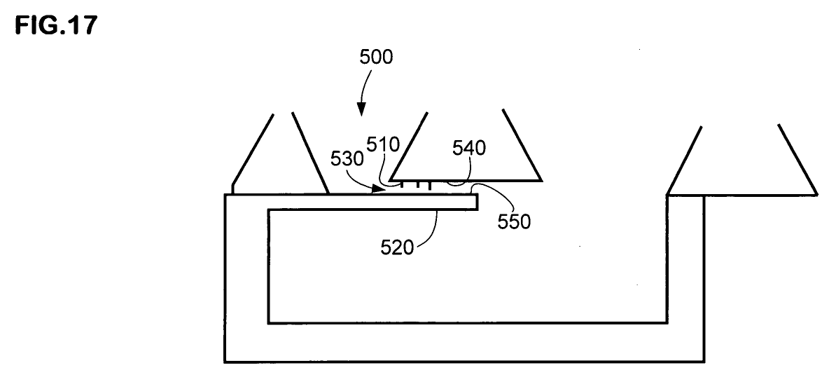

FIG. 17 shows a schematic drawing of an embodiment of a valve 500 that employs the present artificial receptors. The valve can operate as a check valve, for example. A cantilevered member 520 extends over a flow opening 530. Receptors 510 can be coupled to a surface 540 that opposes a surface on the cantilevered member. If the cantilevered member can be urged closed, the present artificial receptors can be coupled to the upper surface 550 of the cantilevered member to support closure of the valve. The present artificial receptors 510 can couple directly to the cantilevered member, or alternatively can couple to a material (not shown) on the cantilever member that has a tendency to bond to the present artificial receptors. Other configurations are possible.

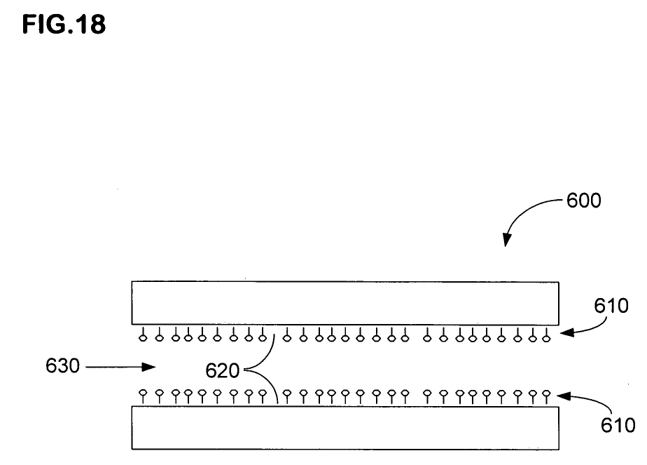

FIG. 18 shows a schematic drawing of a microstructure 600 that includes the present artificial receptors 610. The present artificial receptors 610 can be coupled to interior surfaces 620 of a microchannel 630. A structure such as this receptor-lined microchannel can be used, for example, to remove a test ligand from a fluid that travels down the channel. Other shapes and structures are also possible, including, for example, receptor-lined microtubes. By way of further example, artificial receptors of the present invention could be disposed in or on pores in an otherwise solid membrane.

As described above, artificial receptors can be created according to the invention that have binding affinity for a target substrate. When these artificial receptors are disposed on a nano-scale manipulator, the manipulator can be useful to grip and move objects made of the target substrate because the artificial receptor will selectively bind to the target substrate.

Artificial receptors of the present invention can be used in conjunction with techniques analogous to photolithography that are well known in the art. By way of example, artificial receptors of the present invention can be attached to a substrate by means of a reaction that is catalyzed by a form of radiation such that artificial receptors will be deposited in places that the radiation is directed upon and will not be deposited in other areas where the radiation is not directed. In the manner, techniques analogous to photolithography can be used to precisely place artificial receptors of the present invention where they are desired. These techniques can be used to create nano-scale devices that have artificial receptors deposited on them in precise locations.

Dendrimers are spherical polymeric molecules that consist of a series of chemical shells built on a small core molecule. The core generally consists of an amine core, although sugars and other molecules can be used. With dendrimers, each shell is called a generation. The surface of both full and half generations provide the means of attachment of multiple different functional components. Commercially available dendrimers include polyamidoamine (“PAMAM”) dendrimers and polypropylenimine (“PPI”) dendrimers (Aldrich, Milwaukee, Wis.). Methods for creating dendrimers can be found in U.S. Pat. No. 5,714,166 (Tomalia et al.).

In an embodiment, artificial receptors of the present invention are disposed on a dendrimer. For example, artificial receptors that have specific affinity for a target ligand that is characteristic of a certain type of disease can be disposed on the surface of a dendrimer that is appropriately functionalized to mediate drug delivery in order to help provide site-specific delivery of the drug.

Methods of Making an Artificial Receptor

The present invention relates to a method of making an artificial receptor or a candidate artificial receptor. In an embodiment, this method includes preparing a spot or region on a support, the spot or region including a plurality of building blocks immobilized on the support. The method can include forming a plurality of spots on a solid support, each spot including a plurality of building blocks, and immobilizing (e.g., reversibly) a plurality of building blocks on the solid support in each spot. In an embodiment, an array of such spots is referred to as a heterogeneous building block array.

The method can include mixing a plurality of building blocks and employing the mixture in forming the spot(s). Alternatively, the method can include spotting individual building blocks on the support. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in covalent bonding or noncovalent interactions. Forming spots can yield a microarray of spots of heterogeneous combinations of building blocks, each of which can be a candidate artificial receptor. The method can apply or spot building blocks onto a support in combinations of 2, 3, 4, or more building blocks.

In an embodiment, the present method can be employed to produce a solid support having on its surface a plurality of regions or spots, each region or spot including a plurality of building blocks. For example, the method can include spotting a glass slide with a plurality of spots, each spot including a plurality of building blocks. Such a spot can be referred to as including heterogeneous building blocks. A plurality of spots of building blocks can be referred to as an array of spots.

In an embodiment, the present method includes making a receptor surface. Making a receptor surface can include forming a region on a solid support, the region including a plurality of building blocks, and immobilizing (e.g., reversibly) the plurality of building blocks to the solid support in the region. The method can include mixing a plurality of building blocks and employing the mixture in forming the region or regions. Alternatively, the method can include applying individual building blocks in a region on the support. Forming a region on a support can be accomplished, for example, by soaking a portion of the support with the building block solution. The resulting coating including building blocks can be referred to as including heterogeneous building blocks.

A region including a plurality of building blocks can be independent and distinct from other regions including a plurality of building blocks. In an embodiment, one or more regions including a plurality of building blocks can overlap to produce a region including the combined pluralities of building blocks. In an embodiment, two or more regions including a single building block can overlap to form one or more regions each including a plurality of building blocks. The overlapping regions can be envisioned, for example, as portions of overlap in a Ven diagram, or as portions of overlap in a pattern like a plaid or tweed.

In an embodiment, the method produces a spot or surface with a density of building blocks sufficient to provide interactions of more than one building block with a ligand. That is, the building blocks can be in proximity to one another. Proximity of different building blocks can be detected by determining different (e.g., greater) binding of a test ligand to a spot or surface including a plurality of building blocks compared to a spot or surface including only one of the building blocks.

In an embodiment, the method includes forming an array of heterogeneous spots made from combinations of a subset of the total building blocks and/or smaller groups of the building blocks in each spot. That is, the method forms spots including only, for example, 2 or 3 building blocks, rather than 4 or 5. For example, the method can form spots from combinations of a full set of building blocks (e.g. 81 of a set of 81) in groups of 2 and/or 3. For example, the method can form spots from combinations of a subset of the building blocks (e.g., 25 of the set of 81) in groups of 4 or 5. For example, the method can form spots from combinations of a subset of the building blocks (e.g., 25 of a set of 81) in groups of 2 or 3. The method can include forming additional arrays incorporating building blocks, lead artificial receptors, or structurally similar building blocks.

In an embodiment, the method includes forming an array including one or more spots that function as controls for validating or evaluating binding to artificial receptors of the present invention. In an embodiment, the method includes forming one or more regions, tubes, or wells that function as controls for validating or evaluating binding to artificial receptors of the present invention. Such a control spot, region, tube, or well can include no building block, only a single building block, only functionalized lawn, or combinations thereof.

The method can immobilize (e.g., reversibly) building blocks on supports using known methods for immobilizing compounds of the types employed as building blocks. Coupling building blocks to the support can employ covalent bonding or noncovalent interactions. Suitable noncovalent interactions include interactions between ions, hydrogen bonding, van der Waals interactions, and the like. In an embodiment, the support can be functionalized with moieties that can engage in reversible covalent bonding, moieties that can engage in noncovalent interactions, a mixture of these moieties, or the like.

In an embodiment, the support can be functionalized with moieties that can engage in covalent bonding, e.g., reversible covalent bonding. The present invention can employ any of a variety of the numerous known functional groups, reagents, and reactions for forming reversible covalent bonds. Suitable reagents for forming reversible covalent bonds include those described in Green, T W; Wuts, P G M (1999), Protective Groups in Organic Synthesis Third Edition, Wiley-Interscience, New York, 779 pp. For example, the support can include functional groups such as a carbonyl group, a carboxyl group, a silane group, boric acid or ester, an amine group (e.g., a primary, secondary, or tertiary amine, a hydroxylamine, a hydrazine, or the like), a thiol group, an alcohol group (e.g., primary, secondary, or tertiary alcohol), a diol group (e.g., a 1,2 diol or a 1,3 diol), a phenol group, a catechol group, or the like. These functional groups can form groups with reversible covalent bonds, such as ether (e.g., alkyl ether, silyl ether, thioether, or the like), ester (e.g., alkyl ester, phenol ester, cyclic ester, thioester, or the like), acetal (e.g., cyclic acetal), ketal (e.g., cyclic ketal), silyl derivative (e.g., silyl ether), boronate (e.g., cyclic boronate), amide, hydrazide, imine, carbamate, or the like. Such a functional group can be referred to as a covalent bonding moiety, e.g., a first covalent bonding moiety.

A carbonyl group on the support and an amine group on a building block can form an imine or Schiff's base. The same is true of an amine group on the support and a carbonyl group on a building block. A carbonyl group on the support and an alcohol group on a building block can form an acetal or ketal. The same is true of an alcohol group on the support and a carbonyl group on a building block. A thiol (e.g., a first thiol) on the support and a thiol (e.g., a second thiol) on the building block can form a disulfide.

A carboxyl group on the support and an alcohol group on a building block can form an ester. The same is true of an alcohol group on the support and a carboxyl group on a building block. Any of a variety of alcohols and carboxylic acids can form esters that provide covalent bonding that can be reversed in the context of the present invention. For example, reversible ester linkages can be formed from alcohols such as phenols with electron withdrawing groups on the aryl ring, other alcohols with electron withdrawing groups acting on the hydroxyl-bearing carbon, other alcohols, or the like; and/or carboxyl groups such as those with electron withdrawing groups acting on the acyl carbon (e.g., nitrobenzylic acid, R—CF₂—COOH, R—CCl₂—COOH, and the like), other carboxylic acids, or the like.

In an embodiment, the support, matrix, or lawn can be functionalized with moieties that can engage in noncovalent interactions. For example, the support can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the support, matrix, or lawn includes a charged moiety (e.g., a first charged moiety). Suitable charged moieties include positively charged moieties and negatively charged moieties. Suitable positively charged moieties (e.g., at neutral pH in aqueous compositions) include amines, quaternary ammonium moieties, ferrocene, or the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, hydroxamic acids, or the like.

In an embodiment, the support, matrix, or lawn includes groups that can hydrogen bond (e.g., a first hydrogen bonding group), either as donors or acceptors. The support, matrix, or lawn can include a surface or region with groups that can hydrogen bond. For example, the support, matrix, or lawn can include a surface or region including one or more carboxyl groups, amine groups, hydroxyl groups, carbonyl groups, or the like. Ionic groups can also participate in hydrogen bonding.

In an embodiment, the support, matrix, or lawn includes a lipophilic moiety (e.g., a first lipophilic moiety). Suitable lipophilic moieties include branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. A lipophilic moiety like a quaternary ammonium lipophilic moiety can also include a positive charge.

Artificial Receptors

A candidate artificial receptor, a lead artificial receptor, or a working artificial receptor includes combination of building blocks immobilized (e.g., reversibly) on, for example, a support. An individual artificial receptor can be a heterogeneous building block spot on a slide or a plurality of building blocks coated on a slide, tube, or well. The building blocks can be immobilized through any of a variety of interactions, such as covalent, electrostatic, or hydrophobic interactions. For example, the building block and support or lawn can each include one or more functional groups or moieties that can form covalent, electrostatic, hydrogen bonding, van der Waals, or like interactions.

An array of candidate artificial receptors can be a commercial product sold to parties interested in using the candidate artificial receptors as implements in developing receptors for test ligands of interest. In an embodiment, a useful array of candidate artificial receptors includes at least one glass slide, the at least one glass slide including spots of a predetermined number of combinations of members of a set of building blocks, each combination including a predetermined number of building blocks.

One or more lead artificial receptors can be developed from a plurality of candidate artificial receptors. In an embodiment, a lead artificial receptor includes a combination of building blocks and binds detectable quantities of test ligand upon exposure to, for example, several picomoles of test ligand at a concentration of 1, 0.1, or 0.01 μg/ml, or at 1, 0.1, or 0.01 ng/ml test ligand; at a concentration of 0.01 μg/ml, or at 1, 0.1, or 0.01 ng/ml test ligand; or a concentration of 1, 0.1, or 0.01 ng/ml test ligand.

Artificial receptors, particularly candidate or lead artificial receptors, can be in the form of an array of artificial receptors. Such an array can include, for example, 1.66 million spots, each spot including one combination of 4 building blocks from a set of 81 building blocks. Such an array can include, for example, 28,000 spots, each spot including one combination of 2, 3, or 4 building blocks from a set of 29 building blocks. Each spot is a candidate artificial receptor and a combination of building blocks. The array can also be constructed to include lead artificial receptors. For example, the array of artificial receptors can include combinations of fewer building blocks and/or a subset of the building blocks.

In an embodiment, an array of candidate artificial receptors includes building blocks of general Formula 2 (shown hereinbelow), with RE₁ being B1, B2, B3, B3a, B4, B5, B6, B7, B8, or B9 (shown hereinbelow) and with RE₂ being A1, A2, A3, A3a, A4, A5, A6, A7, A8, or A9 (shown hereinbelow). In an embodiment, the framework is tyrosine.

One or more working artificial receptors can be developed from one or more lead artificial receptors. In an embodiment, a working artificial receptor includes a combination of building blocks and binds categorizing or identifying quantities of test ligand upon exposure to, for example, several picomoles of test ligand at a concentration of 100, 10, 1, 0.1, 0.01, or 0.001 ng/ml test ligand; at a concentration of 10, 1, 0.1, 0.01, or 0.001 ng/ml test ligand; or a concentration of 1, 0.1, 0.01, or 0.001 ng/ml test ligand.

In an embodiment, the artificial receptor of the invention includes a plurality of building blocks coupled to a support. In an embodiment, the plurality of building blocks can include or be building blocks of Formula 2 (shown below). An abbreviation for the building block including a linker, a tyrosine framework, and recognition elements AxBy is TyrAxBy. In an embodiment, a candidate artificial receptor can include combinations of building blocks of formula TyrA1B1, TyrA2B2, TyrA2B4, TyrA2B6, TyrA2B8, TyrA3B3, TyrA4B2, TyrA4B4, TyrA4B6, TyrA4B8, TyrA5B5, TyrA6B2, TyrA6B4, TyrA6B6, TyrA6B8, TyrA7B7, TyrA8B2, TyrA8B4, TyrA8B6, or TyrA8B8.

The present artificial receptors can employ any of a variety of supports to which building blocks or other array materials can be coupled. For example, the support can be glass or plastic; a slide, a tube, or a well; an optical fiber; a nanotube or a buckyball, a nanodevice; a dendrimer, or a scaffold; or the like.

Building Blocks

The present invention relates to building blocks for making or forming candidate artificial receptors. Building blocks can be designed, made, and selected to provide a variety of structural characteristics among a small number of compounds. A building block can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A building block can be bulky or it can be small.

A building block can be visualized as including several components, such as one or more frameworks, one or more linkers, and/or one or more recognition elements. The framework can be covalently coupled to each of the other building block components. The linker can be covalently coupled to the framework. The linker can be coupled to a support through one or more of covalent, electrostatic, hydrogen bonding, van der Waals, or like interactions. The recognition element can be covalently coupled to the framework. In an embodiment, a building block includes a framework, a linker, and a recognition element. In an embodiment, a building block includes a framework, a linker, and two recognition elements.

A description of general and specific features and functions of a variety of building blocks and their synthesis can be found in copending U.S. patent application Ser. Nos. 10/244,727, filed Sep. 16, 2002, Ser. No. 10/813,568, filed Mar. 29, 2004, and Application No. PCT/US03/05328, filed Feb. 19, 2003, each entitled “ARTIFICIAL RECEPTORS, BUILDING BLOCKS, AND METHODS”; U.S. patent application Ser. Nos. 10/812,850 and 10/813,612, and application No. PCT/U.S. 2004/009649, each filed Mar. 29, 2004 and each entitled “ARTIFICIAL RECEPTORS INCLUDING REVERSIBLY IMMOBILIZED BUILDING BLOCKS, THE BUILDING BLOCKS, AND METHODS”; and U.S. Provisional Patent Application No. 60/499,965, filed Sep. 3, 2003, and 60/526,699, filed Dec. 2, 2003, each entitled BUILDING BLOCKS FOR ARTIFICIAL RECEPTORS; the disclosures of which are incorporated herein by reference. These patent documents include, in particular, a detailed written description of: function, structure, and configuration of building blocks, framework moieties, recognition elements, synthesis of building blocks, specific embodiments of building blocks, specific embodiments of recognition elements, and sets of building blocks.

Framework

The framework can be selected for functional groups that provide for coupling to the recognition moiety and for coupling to or being the linking moiety. The framework can interact with the ligand as part of the artificial receptor. In an embodiment, the framework includes multiple reaction sites with orthogonal and reliable functional groups and with controlled stereochemistry. Suitable functional groups with orthogonal and reliable chemistries include, for example, carboxyl, amine, hydroxyl, phenol, carbonyl, and thiol groups, which can be individually protected, deprotected, and derivatized. In an embodiment, the framework has two, three, or four functional groups with orthogonal and reliable chemistries. In an embodiment, the framework has three functional groups. In such an embodiment, the three functional groups can be independently selected, for example, from carboxyl, amine, hydroxyl, phenol, carbonyl, or thiol group. The framework can include alkyl, substituted alkyl, cycloalkyl, heterocyclic, substituted heterocyclic, aryl alkyl, aryl, heteroaryl, heteroaryl alkyl, and like moieties.

A general structure for a framework with three functional groups can be represented by Formula Ia: 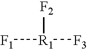
A general structure for a framework with four functional groups can be represented by Formula Ib: 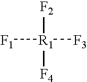
In these general structures: R₁ can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group; and F₁, F₂, F₃, or F₄ can independently be a carboxyl, amine, hydroxyl, phenol, carbonyl, or thiol group. F₁, F₂, F₃, or F₄ can independently be a 1-12, a 1-6, a 1-4 carbon alkyl, substituted alkyl, cycloalkyl, heterocyclic, substituted heterocyclic, aryl alkyl, aryl, heteroaryl, heteroaryl alkyl, or inorganic group substituted with carboxyl, amine, hydroxyl, phenol, carbonyl, or thiol group. F₃ and/or F₄ can be absent.

A variety of compounds fit the formulas and text describing the framework including amino acids, and naturally occurring or synthetic compounds including, for example, oxygen and sulfur functional groups. The compounds can be racemic, optically active, or achiral. For example, the compounds can be natural or synthetic amino acids, α-hydroxy acids, thioic acids, and the like.

Suitable molecules for use as a framework include a natural or synthetic amino acid, particularly an amino acid with a functional group (e.g., third functional group) on its side chain. Amino acids include carboxyl and amine functional groups. The side chain functional group can include, for natural amino acids, an amine (e.g., alkyl amine, heteroaryl amine), hydroxyl, phenol, carboxyl, thiol, thioether, or amidino group. Natural amino acids suitable for use as frameworks include, for example, serine, threonine, tyrosine, aspartic acid, glutamic acid, asparagine, glutamine, cysteine, lysine, arginine, histidine. Synthetic amino acids can include the naturally occurring side chain functional groups or synthetic side chain functional groups which modify or extend the natural amino acids with alkyl, substituted alkyl, cycloalkyl, heterocyclic, substituted heterocyclic, aryl alkyl, aryl, heteroaryl, heteroaryl alkyl, and like moieties as framework and with carboxyl, amine, hydroxyl, phenol, carbonyl, or thiol functional groups. Suitable synthetic amino acids include β-amino acids and homo or β analogs of natural amino acids. In an embodiment, the framework amino acid can be serine, threonine, or tyrosine, e.g., serine or tyrosine, e.g., tyrosine.

Although not limiting to the present invention, a framework amino acid, such as serine, threonine, or tyrosine, with a linker and two recognition elements can be visualized with one of the recognition elements in a pendant orientation and the other in an equatorial orientation, relative to the extended carbon chain of the framework.

All of the naturally occurring and many synthetic amino acids are commercially available. Further, forms of these amino acids derivatized or protected to be suitable for reactions for coupling to recognition element(s) and/or linkers can be purchased or made by known methods (see, e.g., Green, T W; Wuts, P G M (1999), Protective Groups in Organic Synthesis Third Edition, Wiley-Interscience, New York, 779 pp.; Bodanszky, M.; Bodanszky, A. (1994), The Practice of Peptide Synthesis Second Edition, Springer-Verlag, New York, 217 pp.).

Recognition Element

The recognition element can be selected to provide one or more structural characteristics to the building block. The recognition element can interact with the ligand as part of the artificial receptor. For example, the recognition element can provide one or more structural characteristics such as positive charge, negative charge, acid, base, electron acceptor, electron donor, hydrogen bond donor, hydrogen bond acceptor, free electron pair, π electrons, charge polarization, hydrophilicity, hydrophobicity, and the like. A recognition element can be a small group or it can be bulky.

In an embodiment the recognition element can be a 1-12, a 1-6, or a 1-4 carbon alkyl, substituted alkyl, cycloalkyl, heterocyclic, substituted heterocyclic, aryl alkyl, aryl, heteroaryl, heteroaryl alkyl, or like group. The recognition element can be substituted with a group that includes or imparts positive charge, negative charge, acid, base, electron acceptor, electron donor, hydrogen bond donor, hydrogen bond acceptor, free electron pair, π electrons, charge polarization, hydrophilicity, hydrophobicity, and the like.

Recognition elements with a positive charge (e.g., at neutral pH in aqueous compositions) include amines, quaternary ammonium moieties, sulfonium, phosphonium, ferrocene, and the like. Suitable amines include alkyl amines, alkyl diamines, heteroalkyl amines, aryl amines, heteroaryl amines, aryl alkyl amines, pyridines, heterocyclic amines (saturated or unsaturated, the nitrogen in the ring or not), amidines, hydrazines, and the like. Alkyl amines generally have 1 to 12 carbons, e.g., 1-8, and rings can have 3-12 carbons, e.g., 3-8. Suitable alkyl amines include that of formula B9. Suitable heterocyclic or alkyl heterocyclic amines include that of formula A9. Suitable pyridines include those of formulas A5 and B5. Any of the amines can be employed as a quaternary ammonium compound.

Additional suitable quaternary ammonium moieties include trimethyl alkyl quaternary ammonium moieties, dimethyl ethyl alkyl quaternary ammonium moieties, dimethyl alkyl quaternary ammonium moieties, aryl alkyl quaternary ammonium moieties, pyridinium quaternary ammonium moieties, and the like. Recognition elements with a negative charge (e.g., at neutral pH in aqueous compositions) include carboxylates, phenols substituted with strongly electron withdrawing groups (e.g., substituted tetrachlorophenols), phosphates, phosphonates, phosphinates, sulphates, sulphonates, thiocarboxylates, and hydroxamic acids. Suitable carboxylates include alkyl carboxylates, aryl carboxylates, and aryl alkyl carboxylates. Suitable phosphates include phosphate mono-, di-, and tri-esters, and phosphate mono-, di-, and tri-amides. Suitable phosphonates include phosphonate mono- and di-esters, and phosphonate mono- and di-amides (e.g., phosphonamides). Suitable phosphinates include phosphinate esters and amides.

Recognition elements with a negative charge and a positive charge (at neutral pH in aqueous compositions) include sulfoxides, betaines, and amine oxides.

Acidic recognition elements can include carboxylates, phosphates, sulphates, and phenols. Suitable acidic carboxylates include thiocarboxylates. Suitable acidic phosphates include the phosphates listed hereinabove.

Basic recognition elements include amines. Suitable basic amines include alkyl amines, aryl amines, aryl alkyl amines, pyridines, heterocyclic amines (saturated or unsaturated, the nitrogen in the ring or not), amidines, and any additional amines listed hereinabove. Suitable alkyl amines include that of formula B9. Suitable heterocyclic or alkyl heterocyclic amines include that of formula A9. Suitable pyridines include those of formulas A5 and B5.

Recognition elements including a hydrogen bond donor include amines, amides, carboxyls, protonated phosphates, protonated phosphonates, protonated phosphinates, protonated sulphates, protonated sulphinates, alcohols, and thiols. Suitable amines include alkyl amines, aryl amines, aryl alkyl amines, pyridines, heterocyclic amines (saturated or unsaturated, the nitrogen in the ring or not), amidines, ureas, and any other amines listed hereinabove. Suitable alkyl amines include that of formula B9. Suitable heterocyclic or alkyl heterocyclic amines include that of formula A9. Suitable pyridines include those of formulas A5 and B5. Suitable protonated carboxylates, protonated phosphates include those listed hereinabove. Suitable amides include those of formulas A8 and B8. Suitable alcohols include primary alcohols, secondary alcohols, tertiary alcohols, and aromatic alcohols (e.g., phenols). Suitable alcohols include those of formulas A7 (a primary alcohol) and B7 (a secondary alcohol).

Recognition elements including a hydrogen bond acceptor or one or more free electron pairs include amines, amides, carboxylates, carboxyl groups, phosphates, phosphonates, phosphinates, sulphates, sulphonates, alcohols, ethers, thiols, and thioethers. Suitable amines include alkyl amines, aryl amines, aryl alkyl amines, pyridines, heterocyclic amines (saturated or unsaturated, the nitrogen in the ring or not), amidines, ureas, and amines as listed hereinabove. Suitable alkyl amines include that of formula B9. Suitable heterocyclic or alkyl heterocyclic amines include that of formula A9. Suitable pyridines include those of formulas A5 and B5. Suitable carboxylates include those listed hereinabove. Suitable amides include those of formulas A8 and B8. Suitable phosphates, phosphonates and phosphinates include those listed hereinabove. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include alkyl ethers, aryl alkyl ethers. Suitable alkyl ethers include that of formula A6. Suitable aryl alkyl ethers include that of formula A4.

Suitable thioethers include that of formula B6. Recognition elements including uncharged polar or hydrophilic groups include amides, alcohols, ethers, thiols, thioethers, esters, thio esters, boranes, borates, and metal complexes. Suitable amides include those of formulas A8 and B8. Suitable alcohols include primary alcohols, secondary alcohols, tertiary alcohols, aromatic alcohols, and those listed hereinabove. Suitable alcohols include those of formulas A7 (a primary alcohol) and B7 (a secondary alcohol). Suitable ethers include those listed hereinabove. Suitable ethers include that of formula A6. Suitable aryl alkyl ethers include that of formula A4.

Recognition elements including uncharged hydrophobic groups include alkyl (substituted and unsubstituted), alkene (conjugated and unconjugated), alkyne (conjugated and unconjugated), aromatic. Suitable alkyl groups include lower alkyl, substituted alkyl, cycloalkyl, aryl alkyl, and heteroaryl alkyl. Suitable lower alkyl groups include those of formulas A1, A3, A3a, and B1. Suitable aryl alkyl groups include those of formulas A3, A3a, A4, B3, B3a, and B4. Suitable alkyl cycloalkyl groups include that of formula B2. Suitable alkene groups include lower alkene and aryl alkene. Suitable aryl alkene groups include that of formula B4. Suitable aromatic groups include unsubstituted aryl, heteroaryl, substituted aryl, aryl alkyl, heteroaryl alkyl, alkyl substituted aryl, and polyaromatic hydrocarbons. Suitable aryl alkyl groups include those of formulas A3, A3a and B4. Suitable alkyl heteroaryl groups include those of formulas A5 and B5.

Spacer (e.g., small) recognition elements include hydrogen, methyl, ethyl, and the like. Bulky recognition elements include 7 or more carbon or hetero atoms.

Formulas A1-A9 and B1-B9 are:
CH₂CH₃ A1
CH₂CH(CH₃)₂ A2 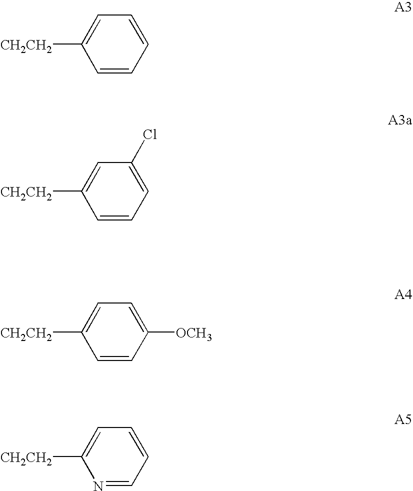 CH₂CH₂—O—CH₃ A6
CH₂CH₂—OH A7
CH₂CH₂—NH—C(O)CH₃ A8 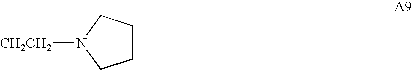 CH₃ B1 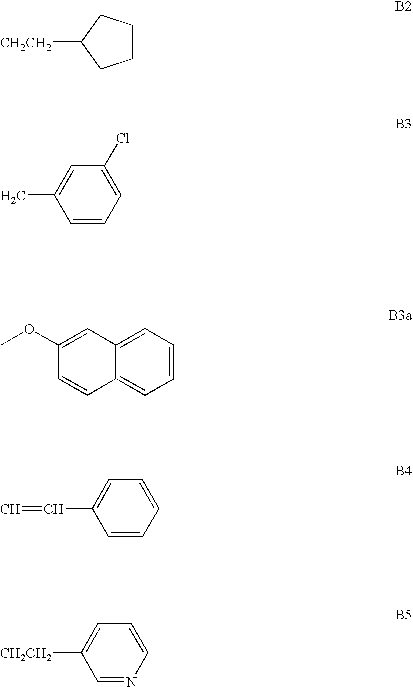 CH₂—S—CH₃ B6
CH₂CH(OH)CH₃ B7
CH₂CH₂C(O)—NH₂ B8
CH₂CH₂CH₂—N—(CH₃)₂ B9

These A and B recognition elements can be called derivatives of, according to a standard reference: A1, ethylamine; A2, isobutylamine; A3, phenethylamine; A4, 4-methoxyphenethylamine; A5,2-(2-aminoethyl)pyridine; A6,2-methoxyethylamine; A7, ethanolamine; A8, N-acetylethylenediamine; A9, 1-(2-aminoethyl)pyrrolidine; B1, acetic acid, B2, cyclopentylpropionic acid; B3,3-chlorophenylacetic acid; B4, cinnamic acid; B5, 3-pyridinepropionic acid; B6, (methylthio)acetic acid; B7,3-hydroxybutyric acid; B8, succinamic acid; and B9,4-(dimethylamino)butyric acid.

In an embodiment, the recognition elements include one or more of the structures represented by formulas A1, A2, A3, A3a, A4, A5, A6, A7, A8, and/or A9 (the A recognition elements) and/or B1, B2, B3, B3a, B4, B5, B6, B7, B8, and/or B9 (the B recognition elements). In an embodiment, each building block includes an A recognition element and a B recognition element. In an embodiment, a group of 81 such building blocks includes each of the 81 unique combinations of an A recognition element and a B recognition element. In an embodiment, the A recognition elements are linked to a framework at a pendant position. In an embodiment, the B recognition elements are linked to a framework at an equatorial position. In an embodiment, the A recognition elements are linked to a framework at a pendant position and the B recognition elements are linked to the framework at an equatorial position.

Although not limiting to the present invention, it is believed that the A and B recognition elements represent the assortment of functional groups and geometric configurations employed by polypeptide receptors. Although not limiting to the present invention, it is believed that the A recognition elements represent six advantageous functional groups or configurations and that the addition of functional groups to several of the aryl groups increases the range of possible binding interactions. Although not limiting to the present invention, it is believed that the B recognition elements represent six advantageous functional groups, but in different configurations than employed for the A recognition elements. Although not limiting to the present invention, it is further believed that this increases the range of binding interactions and further extends the range of functional groups and configurations that is explored by molecular configurations of the building blocks.

In an embodiment, the building blocks including the A and B recognition elements can be visualized as occupying a binding space defined by lipophilicity/hydrophilicity and volume. A volume can be calculated (using known methods) for each building block including the various A and B recognition elements. A measure of lipophilicity/hydrophilicity (logP) can be calculated (using known methods) for each building block including the various A and B recognition elements. Negative values of logP show affinity for water over nonpolar organic solvent and indicate a hydrophilic nature. A plot of volume versus logP can then show the distribution of the building blocks through a binding space defined by size and lipophilicity/hydrophilicity. Reagents that form many of the recognition elements are commercially available. For example, reagents for forming recognition elements A1, A2, A3, A3a, A4, A5, A6, A7, A8, A9 B1, B2, B3, B3a, B4, B5, B6, B7, B8, and B9 are commercially available.

Linkers

The linker is selected to provide a suitable coupling of the building block to a support.

The framework can interact with the ligand as part of the artificial receptor. The linker can also provide bulk, distance from the support, hydrophobicity, hydrophilicity, and like structural characteristics to the building block. Coupling building blocks to the support can employ covalent bonding or noncovalent interactions. Suitable noncovalent interactions include interactions between ions, hydrogen bonding, van der Waals interactions, and the like. In an embodiment, the linker includes moieties that can engage in covalent bonding or noncovalent interactions. In an embodiment, the linker includes moieties that can engage in covalent bonding. Suitable groups for forming covalent and reversible covalent bonds are described hereinabove.

Linkers for Reversibly Immobilizable Building Blocks

The linker can be selected to provide suitable reversible immobilization of the building block on a support or lawn. In an embodiment, the linker forms a covalent bond with a functional group on the framework. In an embodiment, the linker also includes a functional group that can reversibly interact with the support or lawn, e.g., through reversible covalent bonding or noncovalent interactions.

In an embodiment, the linker includes one or more moieties that can engage in reversible covalent bonding. Suitable groups for reversible covalent bonding include those described hereinabove. An artificial receptor can include building blocks reversibly immobilized on the lawn or support through, for example, imine, acetal, ketal, disulfide, ester, or like linkages. Such functional groups can engage in reversible covalent bonding. Such a functional group can be referred to as a covalent bonding moiety, e.g., a second covalent bonding moiety.

In an embodiment, the linker can be functionalized with moieties that can engage in noncovalent interactions. For example, the linker can include functional groups such as an ionic group, a group that can hydrogen bond, or a group that can engage in van der Waals or other hydrophobic interactions. Such functional groups can include cationic groups, anionic groups, lipophilic groups, amphiphilic groups, and the like.

In an embodiment, the present methods and compositions can employ a linker including a charged moiety (e.g., a second charged moiety). Suitable charged moieties include positively charged moieties and negatively charged moieties. Suitable positively charged moieties include amines, quaternary ammonium moieties, sulfonium, phosphonium, ferrocene, and the like. Suitable negatively charged moieties (e.g., at neutral pH in aqueous compositions) include carboxylates, phenols substituted with strongly electron withdrawing groups (e.g., tetrachlorophenols), phosphates, phosphonates, phosphinates, sulphates, sulphonates, thiocarboxylates, and hydroxamic acids.

In an embodiment, the present methods and compositions can employ a linker including a group that can hydrogen bond, either as donor or acceptor (e.g., a second hydrogen bonding group). For example, the linker can include one or more carboxyl groups, amine groups, hydroxyl groups, carbonyl groups, or the like. Ionic groups can also participate in hydrogen bonding.

In an embodiment, the present methods and compositions can employ a linker including a lipophilic moiety (e.g., a second lipophilic moiety). Suitable lipophilic moieties include one or more branched or straight chain C₆₋₃₆ alkyl, C₈₋₂₄ alkyl, C₁₂₋₂₄ alkyl, C₁₂₋₁₈ alkyl, or the like; C₆₋₃₆ alkenyl, C₈₋₂₄ alkenyl, C₁₂₋₂₄ alkenyl, C₁₂₋₁₈ alkenyl, or the like, with, for example, 1 to 4 double bonds; C₆₋₃₆ alkynyl, C₈₋₂₄ alkynyl, C₁₂₋₂₄ alkynyl, C₁₂₋₁₈ alkynyl, or the like, with, for example, 1 to 4 triple bonds; chains with 1-4 double or triple bonds; chains including aryl or substituted aryl moieties (e.g., phenyl or naphthyl moieties at the end or middle of a chain); polyaromatic hydrocarbon moieties; cycloalkane or substituted alkane moieties with numbers of carbons as described for chains; combinations or mixtures thereof; or the like. The alkyl, alkenyl, or alkynyl group can include branching; within chain functionality like an ether group; terminal functionality like alcohol, amide, carboxylate or the like; or the like. In an embodiment the linker includes or is a lipid, such as a phospholipid. In an embodiment, the lipophilic moiety includes or is a 12-carbon aliphatic moiety.

In an embodiment, the linker includes a lipophilic moiety (e.g., a second lipophilic moiety) and a covalent bonding moiety (e.g., a second covalent bonding moiety). In an embodiment, the linker includes a lipophilic moiety (e.g., a second lipophilic moiety) and a charged moiety (e.g., a second charged moiety).

In an embodiment, the linker forms or can be visualized as forming a covalent bond with an alcohol, phenol, thiol, amine, carbonyl, or like group on the framework. Between the bond to the framework and the group participating in or formed by the reversible interaction with the support or lawn, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

For example, suitable linkers can include: the functional group participating in or formed by the bond to the framework, the functional group or groups participating in or formed by the reversible interaction with the support or lawn, and a linker backbone moiety.

The linker backbone moiety can include about 4 to about 48 carbon or heteroatoms, about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms, or the like. The linker backbone can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, mixtures thereof, or like moiety.

In an embodiment, the linker includes a lipophilic moiety, the functional group participating in or formed by the bond to the framework, and, optionally, one or more moieties for forming a reversible covalent bond, a hydrogen bond, or an ionic interaction. In such an embodiment, the lipophilic moiety can have about 4 to about 48 carbons, about 8 to about 14 carbons, about 12 to about 24 carbons, about 16 to about 18 carbons, or the like. In such an embodiment, the linker can include about 1 to about 8 reversible bond/interaction moieties or about 2 to about 4 reversible bond/interaction moieties. Suitable linkers have structures such as (CH₂)ₙCOOH, with n=12-24, n=17-24, or n=16-18.

Additional Embodiments of Linkers

The linker can be selected to provide a suitable covalent coupling of the building block to a support. The framework can interact with the ligand as part of the artificial receptor. The linker can also provide bulk, distance from the support, hydrophobicity, hydrophilicity, and like structural characteristics to the building block. In an embodiment, the linker forms a covalent bond with a functional group on the framework. In an embodiment, before attachment to the support the linker also includes a functional group that can be activated to react with or that will react with a functional group on the support. In an embodiment, once attached to the support, the linker forms a covalent bond with the support and with the framework.

In an embodiment, the linker forms or can be visualized as forming a covalent bond with an alcohol, phenol, thiol, amine, carbonyl, or like group on the framework. The linker can include a carboxyl, alcohol, phenol, thiol, amine, carbonyl, maleimide, or like group that can react with or be activated to react with the support. Between the bond to the framework and the group formed by the attachment to the support, the linker can include an alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

The linker can include a good leaving group bonded to, for example, an alkyl or aryl group. The leaving group being “good” enough to be displaced by the alcohol, phenol, thiol, amine, carbonyl, or like group on the framework. Such a linker can include a moiety represented by the formula: R—X, in which X is a leaving group such as halogen (e.g., —Cl, —Br or —I), tosylate, mesylate, triflate, and R is alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or like moiety.

Suitable linker groups include those of formula: (CH₂)ₙCOOH, with n=1-16, n=2-8, n=2-6, or n=3. Reagents that form suitable linkers are commercially available and include any of a variety of reagents with orthogonal functionality.

Embodiments of Building Blocks

In an embodiment, building blocks can be represented by Formula 2: 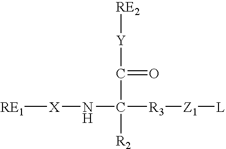
in which: RE₁ is recognition element 1, RE₂ is recognition element 2, and L is a linker. X is absent, C═O, CH₂, NR, NR₂, NH, NHCONH, SCONH, CH═N, or OCH₂NH. In certain embodiments, X is absent or C═O. Y is absent, NH, O, CH₂, or NRCO. In certain embodiments, Y is NH or O. In an embodiment, Y is NH. Z₁ and Z₂ can independently be CH2, O, NH, S, CO, NR, NR₂, NHCONH, SCONH, CH═N, or OCH₂NH. In an embodiment, Z₁ and/or Z₂ can independently be O. Z₂ is optional. R₂ is H, CH₃, or another group that confers chirality on the building block and has size similar to or smaller than a methyl group. R₃ is CH₂; CH₂-phenyl; CHCH₃; (CH₂)ₙ with n=2-3; or cyclic alkyl with 3-8 carbons, e.g., 5-6 carbons, phenyl, naphthyl. In certain embodiments, R₃ is CH₂ or CH₂-phenyl.

RE₁ is B1, B2, B3, B3a, B4, B5, B6, B7, B8, B9, A1, A2, A3, A3a, A4, A5, A6, A7, A8, or A9. In certain embodiments, RE₁ is B1, B2, B3, B3a, B4, B5, B6, B7, B8, or B9. RE₂ is A1, A2, A3, A3a, A4, A5, A6, A7, A8, A9, B1, B2, B3, B3a, B4, B5, B6, B7, B8, or B9. In certain embodiments, RE₂ is A1, A2, A3, A3a, A4, A5, A6, A7, A8, or A9. In an embodiment, RE₁ can be B2, B3a, B4, B5, B6, B7, or B8. In an embodiment, RE₂ can be A2, A3a, A4, A5, A6, A7, or A8.

In an embodiment, L is the functional group participating in or formed by the bond to the framework (such groups are described herein), the functional group or groups participating in or formed by the reversible interaction with the support or lawn (such groups are described herein), and a linker backbone moiety. In an embodiment, the linker backbone moiety is about 4 to about 48 carbon or heteroatom alkyl, substituted alkyl, cycloalkyl, heterocyclic, substituted heterocyclic, aryl alkyl, aryl, heteroaryl, heteroaryl alkyl, ethoxy or propoxy oligomer, a glycoside, or mixtures thereof; or about 8 to about 14 carbon or heteroatoms, about 12 to about 24 carbon or heteroatoms, about 16 to about 18 carbon or heteroatoms, about 4 to about 12 carbon or heteroatoms, about 4 to about 8 carbon or heteroatoms.

In an embodiment, the L is the functional group participating in or formed by the bond to the framework (such groups are described herein) and a lipophilic moiety (such groups are described herein) of about 4 to about 48 carbons, about 8 to about 14 carbons, about 12 to about 24 carbons, about 16 to about 18 carbons. In an embodiment, this L also includes about 1 to about 8 reversible bond/interaction moieties (such groups are described herein) or about 2 to about 4 reversible bond/interaction moieties. In an embodiment, L is (CH₂)ₙCOOH, with n=12-24, n=17-24, or n=16-18. In an embodiment, L is (CH₂)ₙCOOH, with n=1-16, n=2-8, n=4-6, or n=3.

Building blocks including an A and/or a B recognition element, a linker, and an amino acid framework can be made by methods illustrated in general Scheme 1.

Techniques for Using Artificial Receptors

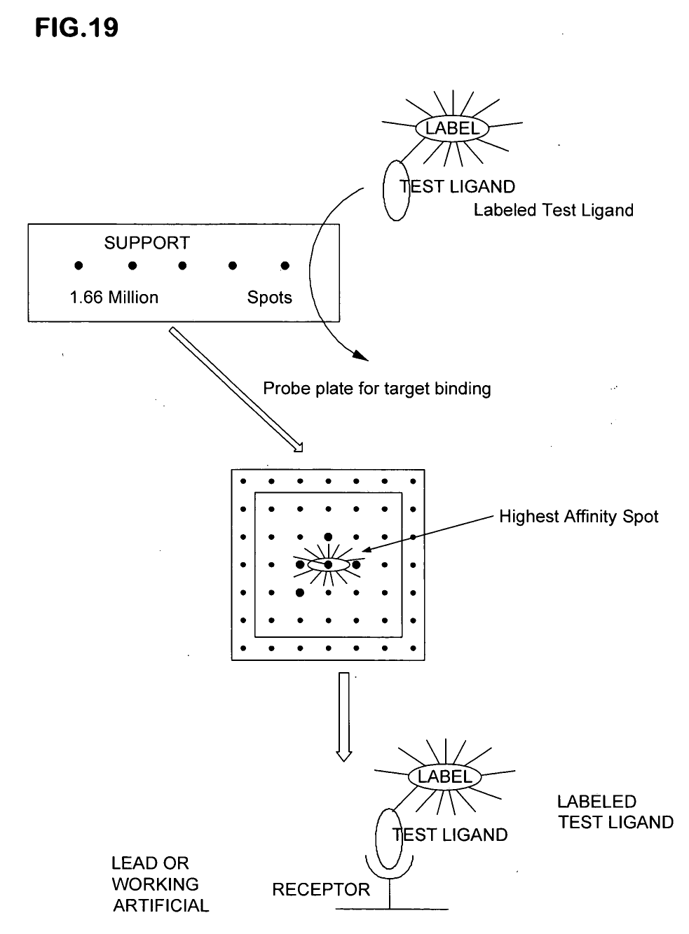

The present invention includes a method of using artificial receptors. The present invention includes a method of screening candidate artificial receptors to find lead artificial receptors that bind a particular test ligand. Detecting test ligand bound to a candidate artificial receptor can be accomplished using known methods for detecting binding to arrays on a slide or to coated tubes or wells. For example, the method can employ test ligand labeled with a detectable label, such as a fluorophore or an enzyme that produces a detectable product. Alternatively, the method can employ an antibody (or other binding agent) specific for the test ligand and including a detectable label. One or more of the spots that are labeled by the test ligand or that are more or most intensely labeled with the test ligand are selected as lead artificial receptors. The degree of labeling can be evaluated by evaluating the signal strength from the label. The amount of signal can be directly proportional to the amount of label and binding. FIG. 19 provides a schematic illustration of an embodiment of this process.

According to the present method, screening candidate artificial receptors against a test ligand can yield one or more lead artificial receptors. One or more lead artificial receptors can be a working artificial receptor. That is, the one or more lead artificial receptors can be useful for detecting the ligand of interest as is. The method can then employ the one or more artificial receptors as a working artificial receptor for monitoring or detecting the test ligand. Alternatively, the one or more lead artificial receptors can be employed in the method for developing a working artificial receptor. For example, the one or more lead artificial receptors can provide structural or other information useful for designing or screening for an improved lead artificial receptor or a working artificial receptor. Such designing or screening can include making and testing additional candidate artificial receptors including combinations of a subset of building blocks, a different set of building blocks, or a different number of building blocks.

The present invention includes a method of screening candidate artificial receptors to find lead artificial receptors that bind a particular test ligand. The method can include allowing movement of the building blocks that make up the artificial receptors. Movement of building blocks can include mobilizing the building block to move along or on the support and/or to leave the support and enter a fluid (e.g., liquid) phase separate from the support or lawn.

In an embodiment, building blocks can be mobilized to move along or on the support (translate or shuffle). Such translation can be employed, for example, to allow building blocks already bound to a test ligand to rearrange into a lower energy or tighter binding configuration still bound to the test ligand. Such translation can be employed, for example, to allow the ligand access to building blocks that are on the support but not bound to the ligand. These building blocks can translate into proximity with and bind to a test ligand.

Building blocks can be induced to move along or on the support or to be reversibly immobilized on the support through any of a variety of mechanisms. For example, inducing mobility of building blocks can include altering the conditions of the support or lawn. That is, altering the conditions can reverse the immobilization of the building blocks, thus mobilizing them. Reversibly immobilizing the building blocks after they have moved can include, for example, returning to the previous conditions. Suitable alterations of conditions include changing pH, changing temperature, changing polarity or hydrophobicity, changing ionic strength, changing nucleophilicity or electrophilicity (e.g. of solvent or solute), and the like.

A building block reversibly immobilized by hydrophobic interactions can be mobilized by increasing the temperature, by exposing the surface, lawn, or building block to a more hydrophobic solvent (e.g., an organic solvent or a surfactant), or by reducing ionic strength around the building block. In an embodiment, the organic solvent includes acetonitrile, acetic acid, an alcohol, tetrahydrofuran (THF), dimethylformamide (DMF), hydrocarbons such as hexane or octane, acetone, chloroform, methylene chloride, or the like, or mixture thereof. In an embodiment, the surfactant includes a nonionic surfactant, such as a nonylphenol ethoxylate, or the like. A building block that is mobile on a support can be reversibly immobilized by hydrophobic interactions, for example, by decreasing the temperature, exposing the surface, lawn, or building block to a more hydrophilic solvent (e.g., an aqueous solvent) or increased ionic strength.

A building block reversibly immobilized by hydrogen bonding can be mobilized by increasing the ionic strength, concentration of hydrophilic solvent, or concentration of a competing hydrogen bonder in the environs of the building block. A building block that is mobile on a support can be reversibly immobilized through an electrostatic interaction by decreasing ionic strength of the hydrophilic solvent, or the like.

A building block reversibly immobilized by an electrostatic interaction can be mobilized by increasing the ionic strength in the environs of the building block. Increasing ionic strength can disrupt electrostatic interactions. A building block that is mobile on a support can be reversibly immobilized through an electrostatic interaction by decreasing ionic strength.

A building block reversibly immobilized by an imine, acetal, or ketal bond can be mobilized by decreasing the pH or increasing concentration of a nucleophilic catalyst in the environs of the building block. In an embodiment, the pH is about 1 to about 4. Imines, acetals, and ketals undergo acid catalyzed hydrolysis. A building block that is mobile on a support can be reversibly immobilized by a reversible covalent interaction, such as by forming an imine, acetal, or ketal bond, by increasing the pH.

In an embodiment, building blocks can be mobilized to leave the support and enter a fluid (e.g., liquid) phase separate from the support or lawn (exchange). For example, building blocks can be exchanged onto and/or off of the support. Exchange can be employed, for example, to allow building blocks on a support but not bound to a test ligand to be removed from the support. Exchange can be employed, for example, to add additional building blocks to the support. The added building blocks can have structures selected based on knowledge of the structures of the building blocks in artificial receptors that bind the test ligand. The added building blocks can have structures selected to provide additional structural diversity. The added building blocks can include all of the building blocks.

A building block reversibly immobilized by hydrophobic interactions can be released from the support by, for example, raising the temperature, e.g., of the support and/or artificial receptor. For example, the hydrophobic interactions (e.g., the hydrophobic group on the support or lawn and on the building block) can be selected to provide immobilized building block at about room temperature or below and release can be accomplished at a temperature above room temperature. For example, the hydrophobic interactions can be selected to provide immobilized building block at about refrigerator temperature (e.g., 4° C.) or below and release can be accomplished at a temperature of, for example, room temperature or above. By way of further example, a building block can be reversibly immobilized by hydrophobic interactions, for example, by contacting the surface or artificial receptor with a fluid containing the building block and that is at or below room temperature.

A building block reversibly immobilized by hydrophobic interactions can be released from the support by, for example, contacting the artificial receptor with a sufficiently hydrophobic fluid (e.g., an organic solvent or a surfactant). In an embodiment, the organic solvent includes acetonitrile, acetic acid, an alcohol, tetrahydrofuran (THF), dimethylformamide (DMF), hydrocarbons such as hexane or octane, acetone, chloroform, methylene chloride, or the like, or mixture thereof. In an embodiment, the surfactant includes a nonionic surfactant, such as a nonylphenol ethoxylate, or the like. Such reversible immobilization can also be effected by contacting the surface or artificial receptor with a hydrophilic solvent and allowing the somewhat lipophilic building block to partition on to the hydrophobic surface or lawn.

A building block reversibly immobilized by an imine, acetal, or ketal bond can be released from the support by, for example, contacting the artificial receptor with fluid having an acid pH or including a nucleophilic catalyst. In an embodiment, the pH is about 1 to about 4. A building block can be reversibly immobilized by a reversible covalent interaction, such as by forming an imine, acetal, or ketal bond, by contacting the surface or artificial receptor with fluid having a neutral or basic pH.

A building block reversibly immobilized by an electrostatic interaction can be released by, for example, contacting the artificial receptor with fluid having sufficiently high ionic strength to disrupt the electrostatic interaction. A building block can be reversibly immobilized through an electrostatic interaction by contacting the surface or artificial receptor with fluid having ionic strength that promotes electrostatic interaction between the building block and the support and/or lawn.

Test Ligands

The test ligand can be any ligand for which binding to an array or surface can be detected. The test ligand can be a pure compound, a mixture, or a “dirty” mixture containing a natural product or pollutant. Such dirty mixtures can be tissue homogenate, biological fluid, soil sample, water sample, or the like.

Test ligands include prostate specific antigen, other cancer markers, insulin, warfarin, other anti-coagulants, cocaine, other drugs-of-abuse, markers for E. coli, markers for Salmonella sp., markers for other food-borne toxins, food-borne toxins, markers for Smallpox virus, markers for anthrax, markers for other possible toxic biological agents, pharmaceuticals and medicines, pollutants and chemicals in hazardous waste, toxic chemical agents, markers of disease, pharmaceuticals, pollutants, biologically important cations (e.g., potassium or calcium ion), peptides, carbohydrates, enzymes, bacteria, viruses, mixtures thereof, and the like. In certain embodiments, the test ligand can be at least one of small organic molecules, inorganic/organic complexes, metal ion, mixture of proteins, protein, nucleic acid, mixture of nucleic acids, mixtures thereof, and the like. Suitable test ligands include any compound or category of compounds described elsewhere in this document as being a test ligand, including, for example, the microbes, proteins, cancer cells, drugs of abuse, and the like.

### EXAMPLES

### Example 1

### Synthesis of Building Blocks

Selected building blocks representative of the alkyl-aromatic-polar span of the an embodiment of the building blocks were synthesized and demonstrated effectiveness of these building blocks for making candidate artificial receptors. These building blocks were made on a framework that can be represented by tyrosine and included numerous recognition element pairs. These recognition element pairs include enough of the range from alkyl, to aromatic, to polar to represent a significant degree of the interactions and functional groups of the full set of 81 such building blocks.

Synthesis

Building block synthesis employed a general procedure outlined in Scheme 7, which specifically illustrates synthesis of a building block on a tyrosine framework with recognition element pair A4B4. This general procedure was employed for synthesis of building blocks including TyrA1B1 [1-1], TyrA2B2, TyrA2B4, TyrA2B6, TyrA2B8, TyrA4B2, TyrA4B4, TyrA4B6, TyrA4B8, TyrA6B2, TyrA6B4, TyrA6B6, TyrA6B8, TyrA8B2, TyrA8B4, TyrA8B6, TyrA8B8, and TyrA9B9, respectively. 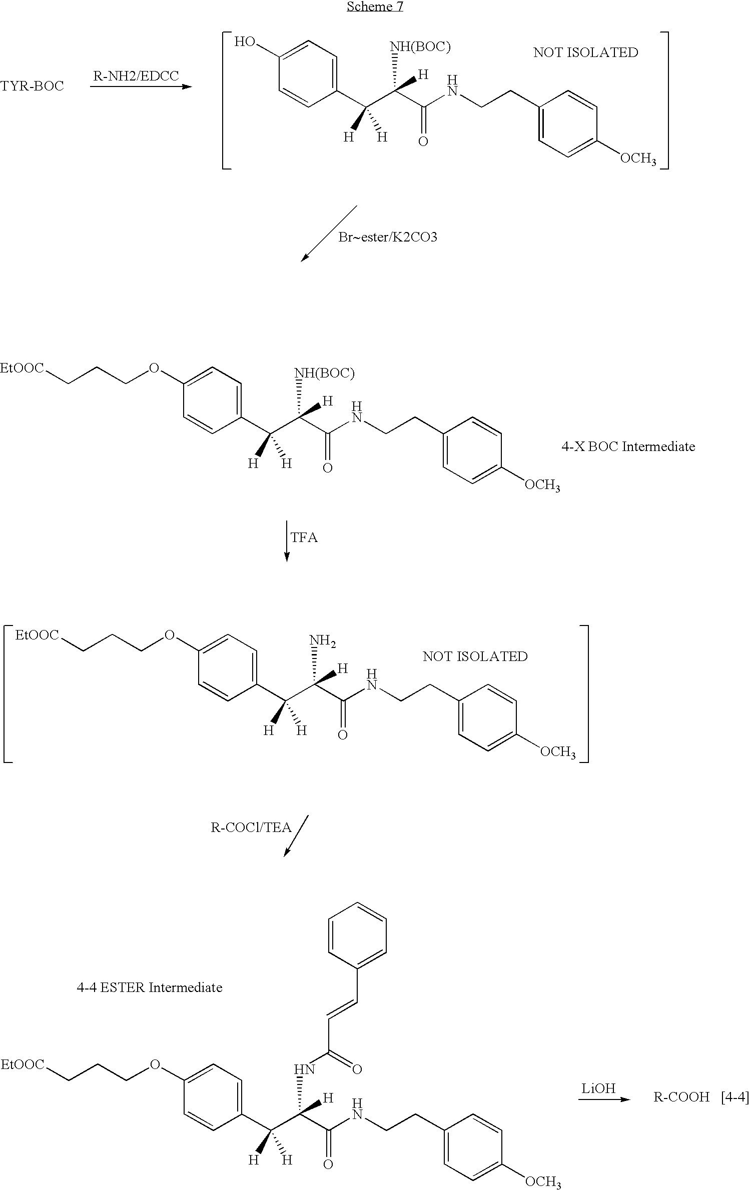
Results

Synthesis of the desired building blocks proved to be generally straightforward. These syntheses illustrate the relative simplicity of preparing the building blocks with 2 recognition elements having different structural characteristics or structures (e.g. A4B2, A6B3, etc.) once the building blocks with corresponding recognition elements (e.g. A2B2, A4B4, etc) have been prepared via their X BOC intermediate.

The conversion of one of these building blocks to a building block with a lipophilic linker can be accomplished by reacting the activated building block with, for example, dodecyl amine.

### Example 2

### Preparation and Evaluation of Microarrays of Candidate Artificial Receptors

Microarrays of candidate artificial receptors were made and evaluated for binding several protein ligands. The results obtained demonstrate the 1) the simplicity with which microarrays of candidate artificial receptors can be prepared, 2) binding affinity and binding pattern reproducibility, 3) significantly improved binding for building block heterogeneous receptor environments when compared to the respective homogeneous controls, and 4) ligand distinctive binding patterns (e.g., working receptor complexes).

Materials and Methods

Building blocks were synthesized and activated as described in Example 1. The building blocks employed in this example were TyrA1B1 [1-1], TyrA2B2, TyrA2B4, TyrA2B6, TyrA4B2, TyrA4B4, TyrA4B6, TyrA6B2, TyrA6B4, and TyrA6B6. The abbreviation for the building block including a linker, a tyrosine framework, and recognition elements AxBy is TyrAxBy.

Microarrays for the evaluation of the 130 n=2 and n=3, and for evaluation of the 273 n=2, n=3, and n=4, candidate receptor environments were prepared as follows by modifications of known methods. As used herein, “n” is the number of different building blocks employed in a receptor environment. Briefly: Amine modified (amine “lawn”; SuperAmine Microarray plates) microarray plates were purchased from Telechem Inc., Sunnyvale, Calif. (www.arrayit.com). These plates were manufactured specifically for microarray preparation and had a nominal amine load of 2-4 amines per square nm according to the manufacturer. The CAM microarrays were prepared using a pin microarray spotter instrument from Telechem Inc. (SpotBot™ Arrayer) typically with 200 um diameter spotting pins from Telechem Inc. (Stealth Micro Spotting Pins, SMP6) and 400-420 um spot spacing.

The 9 building blocks were activated in aqueous dimethylformamide (DMF) solution as described above. For preparing the 384-well feed plate, the activated building block solutions were diluted 10-fold with a solution of DMF/H₂O/PEG400 (90/10/10, v/v/v; PEG400 is polyethylene glycol nominal 400 FW, Aldrich Chemical Co., Milwaukee, Wis.). These stock solutions were aliquotted (10 μl per aliquot) into the wells of a 384-well microwell plate (Telechem Inc.). A separate series of controls were prepared by aliquotting 10 μl of building block with either 10 μl or 20 μl of the activated [1-1] solution. The plate was covered with aluminum foil and placed on the bed of a rotary shaker for 15 minutes at 1,000 RPM. This master plate was stored covered with aluminum foil at −20° C. when not in use.

For preparing the 384-well SpotBot™ plate, a well-to-well transfer (e.g. A-1 to A-1, A-2 to A-2, etc.) from the feed plate to a second 384-well plate was performed using a 4 μl transfer pipette. This plate was stored tightly covered with aluminum foil at −20° C. when not in use. The SpotBot™ was used to prepare up to 13 microarray plates per run using the 4 μl microwell plate. The SpotBot™ was programmed to spot from each microwell in quadruplicate. The wash station on the SpotBot™ used a wash solution of EtOH/H₂O (20/80, v/v). This wash solution was also used to rinse the microarrays on completion of the SpotBot™ printing run. The plates were given a final rinse with deionized (DI) water, dried using a stream of compressed air, and stored at room temperature.

Certain of the microarrays were further modified by reacting the remaining amines with succinic anhydride to form a carboxylate lawn in place of the amine lawn.

The following test ligands and labels were used in these experiments:

1) r-Phycoerythrin, a commercially available and intrinsically fluorescent protein with a FW of 2,000,000.

2) Ovalbumin labeled with the Alexa™ fluorophore (Molecular Probes Inc., Eugene, Oreg.).

3) BSA, bovine serum albumin, labeled with activated Rhodamine (Pierce Chemical, Rockford, Ill.) using the known activated carboxylprotocol. BSA has a FW of 68,000; the material used for this study had ca. 1.0 rhodamine per BSA.

4) Horseradish peroxidase (HRP) modified with extra amines and labeled as the acetamide derivative or with a 2,3,7,8-tetrachlorodibenzodixoin derivative were available through known methods. Fluorescence detection of these HRP conjugates was based on the Alexa 647-tyramide kit available from Molecular Probes, Eugene, Oreg.

5) Cholera toxin labeled with the Alexa™ fluorophore (Molecular Probes Inc., Eugene, Oreg.).

Microarray incubation and analysis was conducted as follows: For test ligand incubation with the microarrays, solutions (e.g. 500 μl) of the target proteins in PBS-T (PBS with 20 μl/L of Tween-20) at typical concentrations of 10, 1.0 and 0.1 μg/ml were placed onto the surface of a microarray and allowed to react for, e.g., 30 minutes. The microarray was rinsed with PBS-T and DI water and dried using a stream of compressed air.

The incubated microarray was scanned using an Axon Model 4200A Fluorescence Microarray Scanner (Axon Instruments, Union City, Calif.). The Axon scanner and its associated software produce a false color 16-bit image of the fluorescence intensity of the plate. This 16-bit data is integrated using the Axon software to give a Fluorescence Units value (range 0-65,536) for each spot on the microarray. This data is then exported into an Excel file (Microsoft) for further analysis including mean, standard deviation and coefficient of variation calculations.

Results

The CARA™: Combinatorial Artificial Receptor Array™ concept has been demonstrated using a microarray format. A CARA microarray based on N=9 building blocks was prepared and evaluated for binding to several protein and substituted protein ligands. This microarray included 144 candidate receptors (18 n=1 controls plus 6 blanks; 36 n=2 candidate receptors; 84 n=3 candidate receptors). This microarray demonstrated: 1) the simplicity of CARA microarray preparation, 2) binding affinity and binding pattern reproducibility, 3) significantly improved binding for building block heterogeneous receptor environments when compared to the respective homogeneous controls, and 4) ligand distinctive binding patterns.

Reading the Arrays

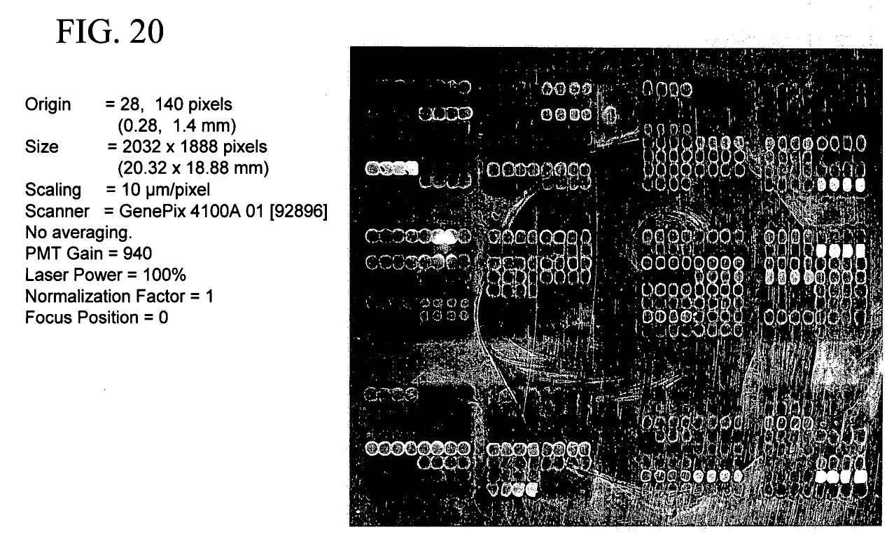

A typical false color/gray scale image of a microarray that was incubated with 2.0 μg/ml r-phycoerythrin is shown in FIG. 20. This image illustrates that the processes of both preparing the microarray and probing it with a protein test ligand produced the expected range of binding as seen in the visual range of relative fluorescence from dark to bright spots.

The starting point in analysis of the data was to take the integrated fluorescence units data for the array of spots and normalize to the observed value for the [1-1] building block control. Subsequent analysis included mean, standard deviation and coefficient of variation calculations. Additionally, control values for homogeneous building blocks were obtained from the building block plus [1-1] data.

First Set of Experiments

The following protein ligands were evaluated for binding to the candidate artificial receptors in the microarray. The resulting Fluorescence Units versus candidate receptor environment data is presented in both a 2D format where the candidate receptors are placed along the X-axis and the Fluorescence Units are shown on the Y-axis and a 3D format where the Candidate Receptors are placed in an X-Y format and the Fluorescence Units are shown on the Z-axis. A key for the composition of each spot was developed (not shown). A key for the building blocks in each of the 2D and 3D representations of the results was also developed (not shown). The data presented are for 1-2 μg/ml protein concentrations.

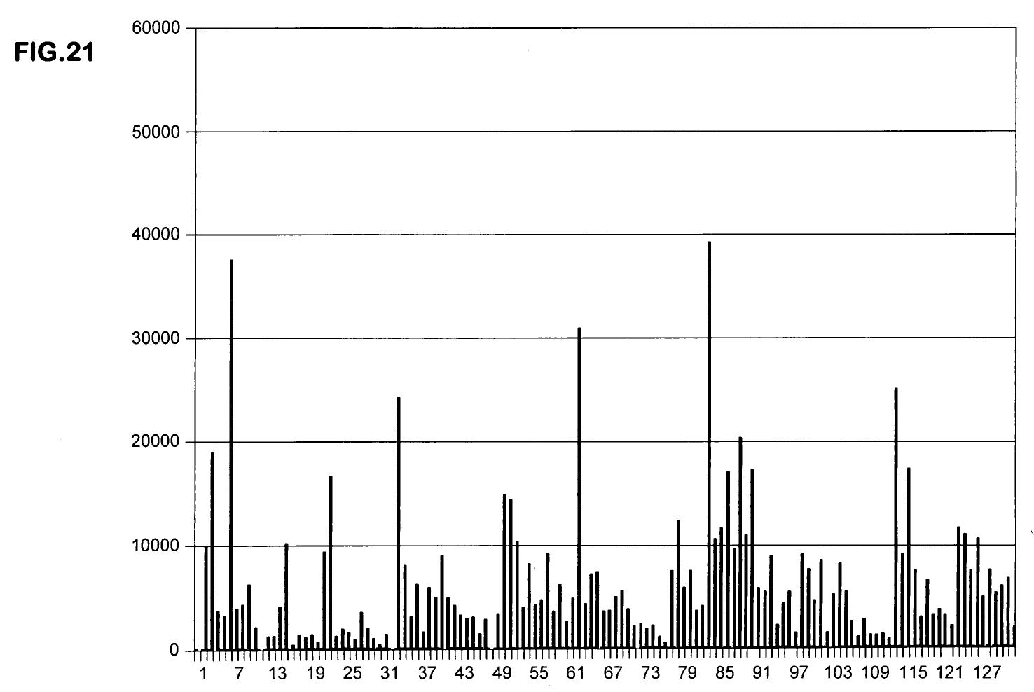

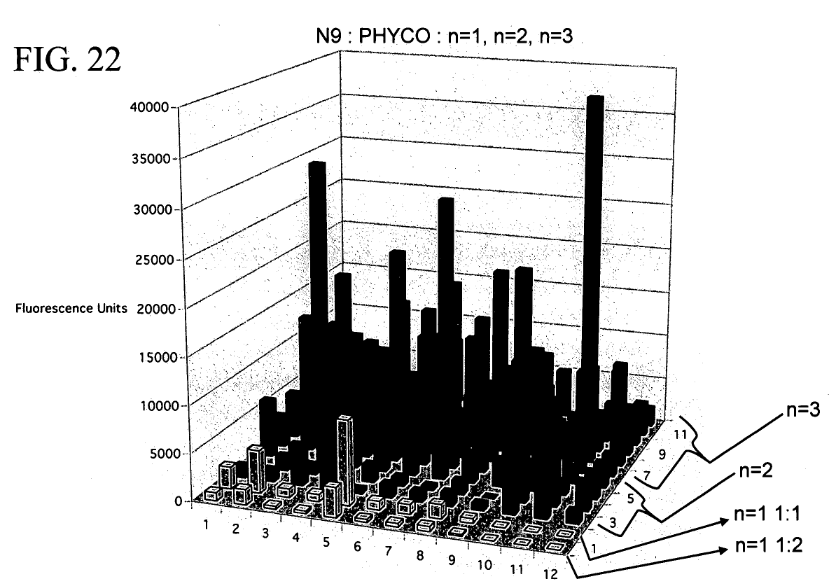

FIGS. 21 and 22 illustrate binding data for r-phycoerythrin (intrinsic fluorescence).

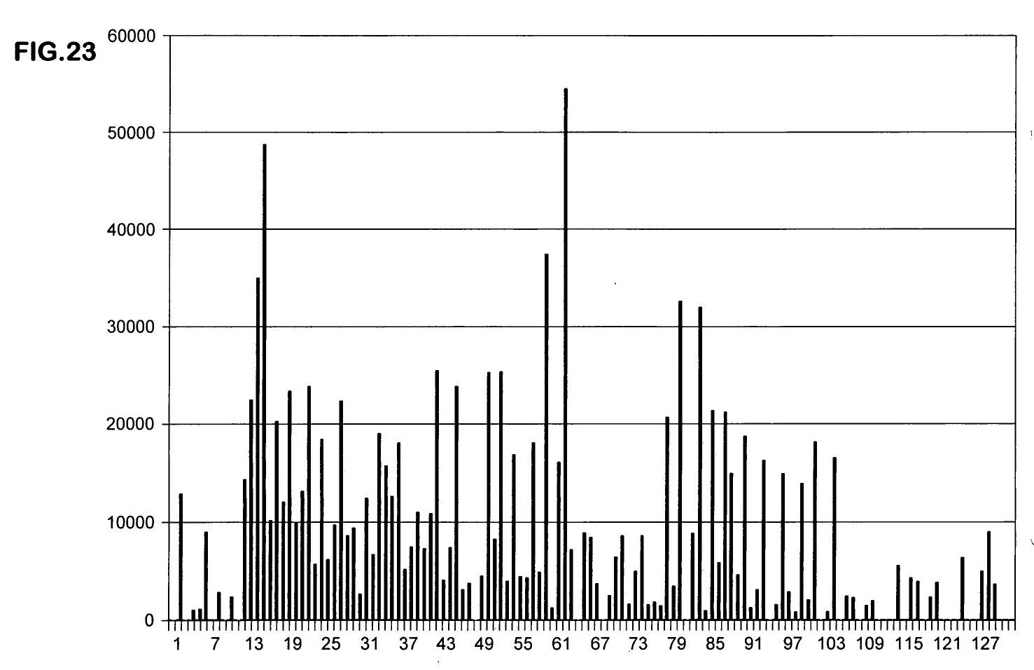

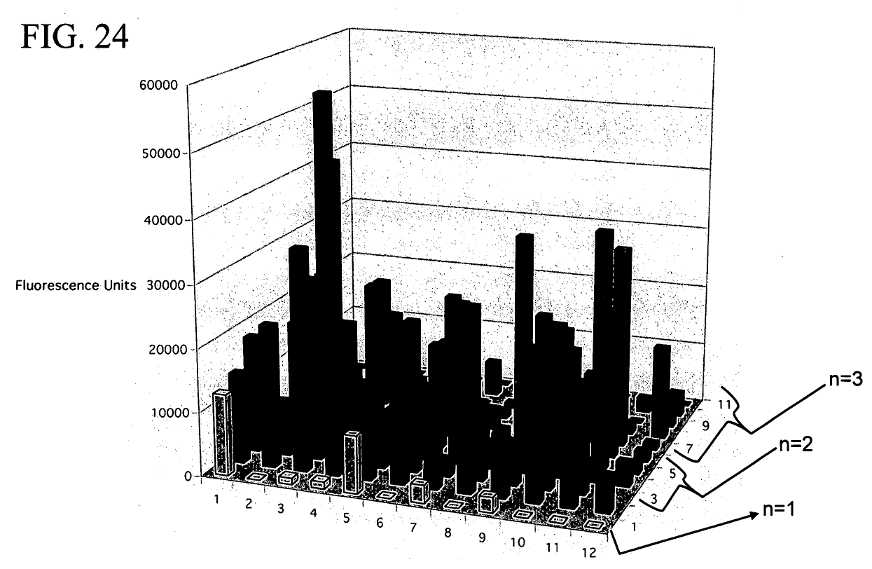

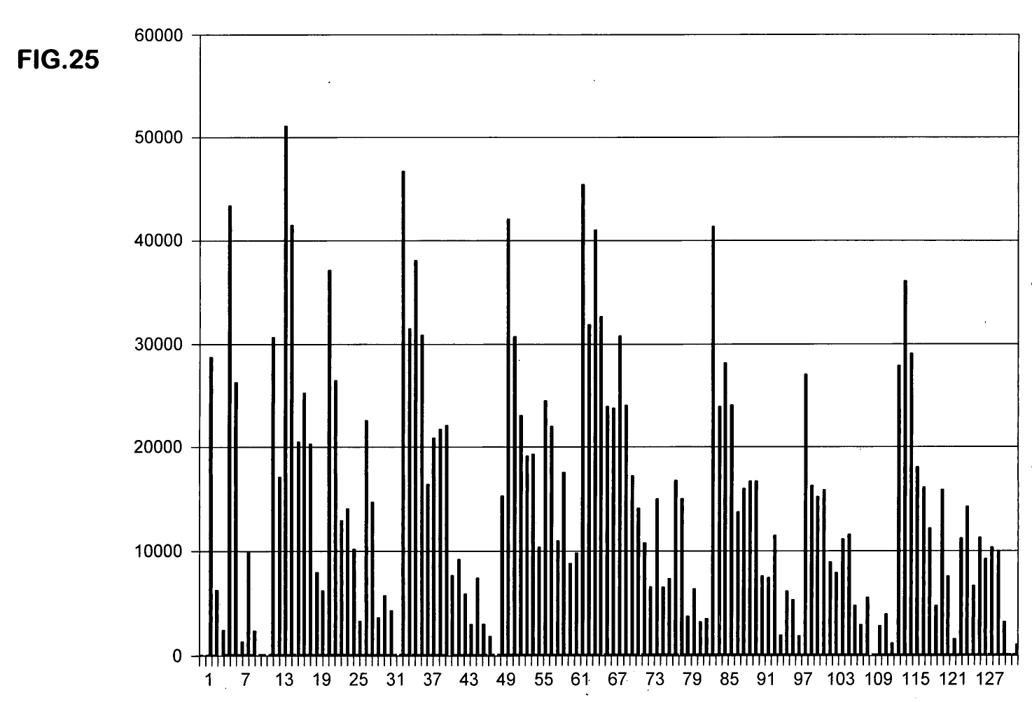

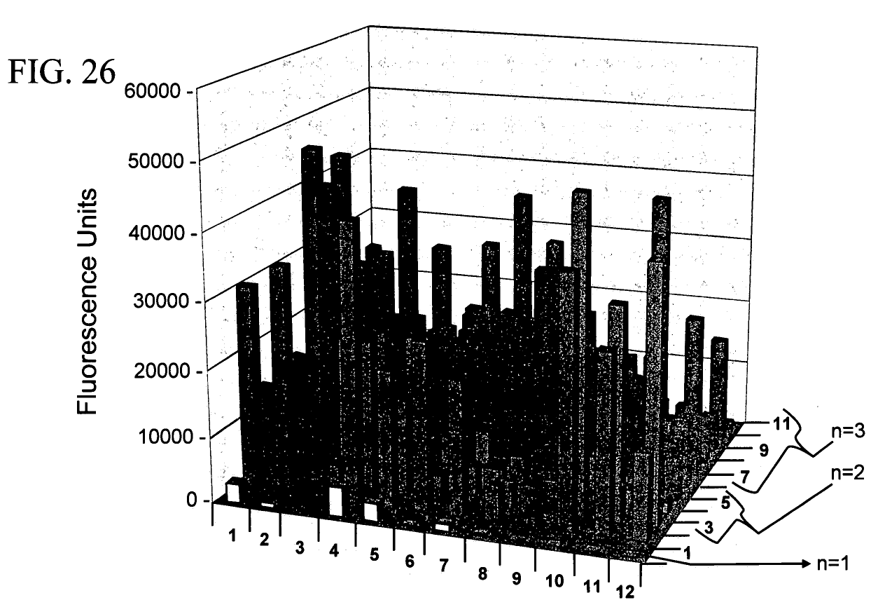

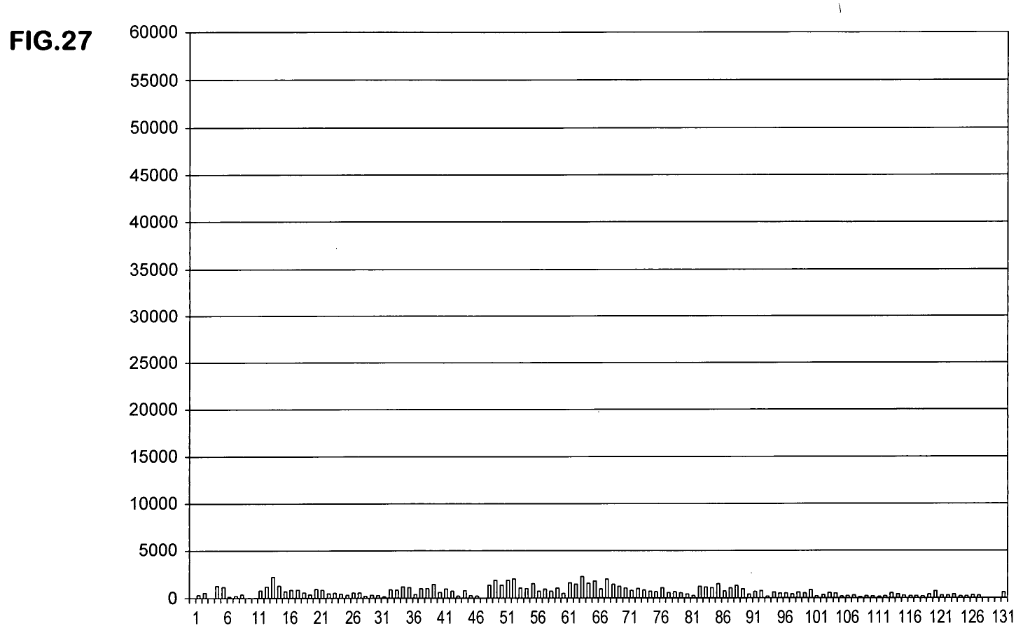

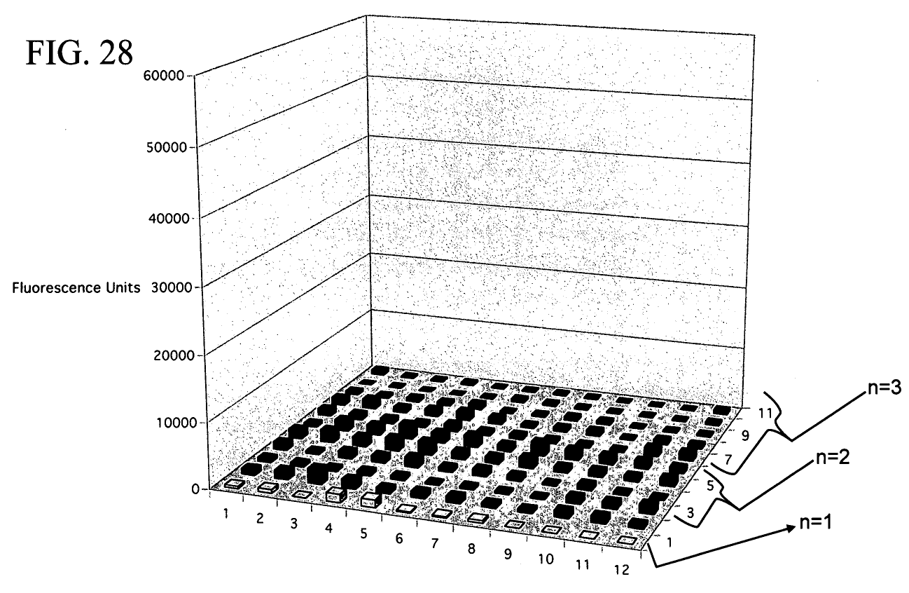

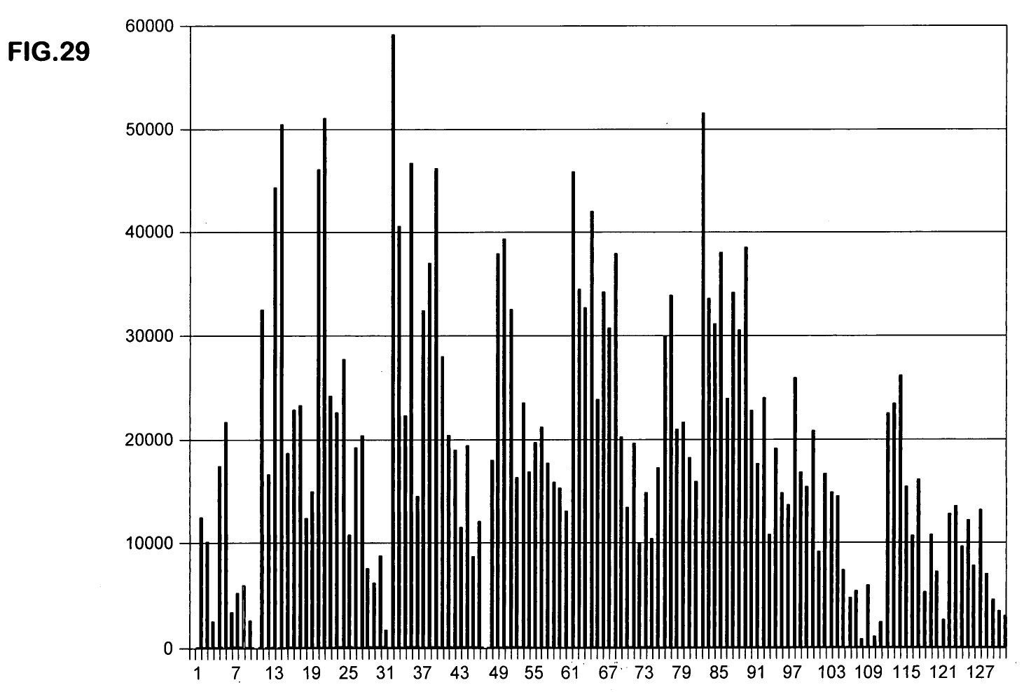

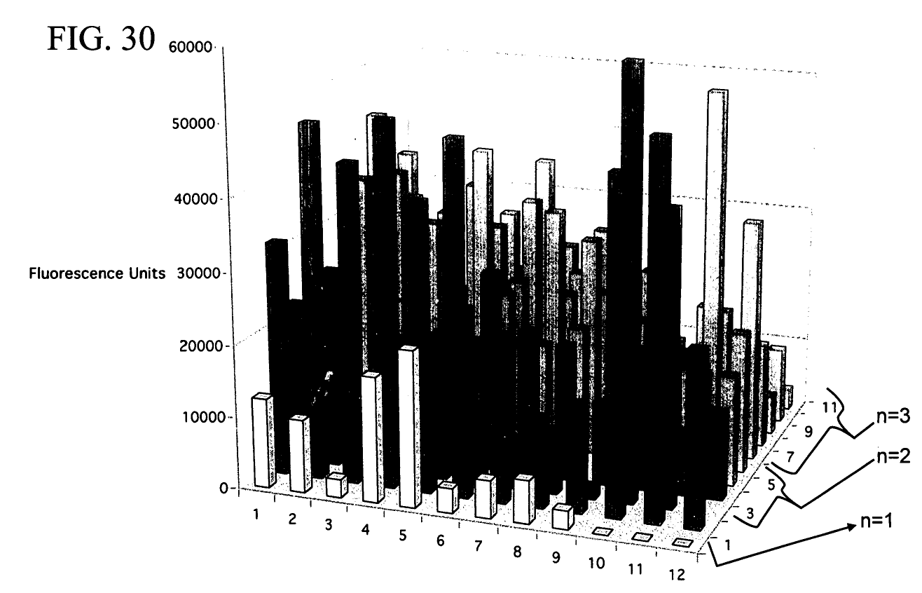

FIGS. 23 and 24 illustrate binding data for ovalbumin (commercially available with fluorescence label). FIGS. 25 and 26 illustrate binding data for bovine serum albumin (labeled with rhodamine). FIGS. 27 and 28 illustrate binding data for HRP-NH-Ac (fluorescent tyramide read-out). FIGS. 29 and 30 illustrate binding data for HRP-NH-TCDD (fluorescent tyramide read-out).

These results demonstrate not only the application of the CARA microarray to candidate artificial receptor evaluation but also a few of the many read-out methods (e.g. intrinsic fluorescence, fluorescently labeled, in situ fluorescence labeling) which can be utilized for high throughput candidate receptor evaluation.

The evaluation of candidate receptors benefits from reproducibility. The following results demonstrate that the present microarrays provided reproducible ligand binding.

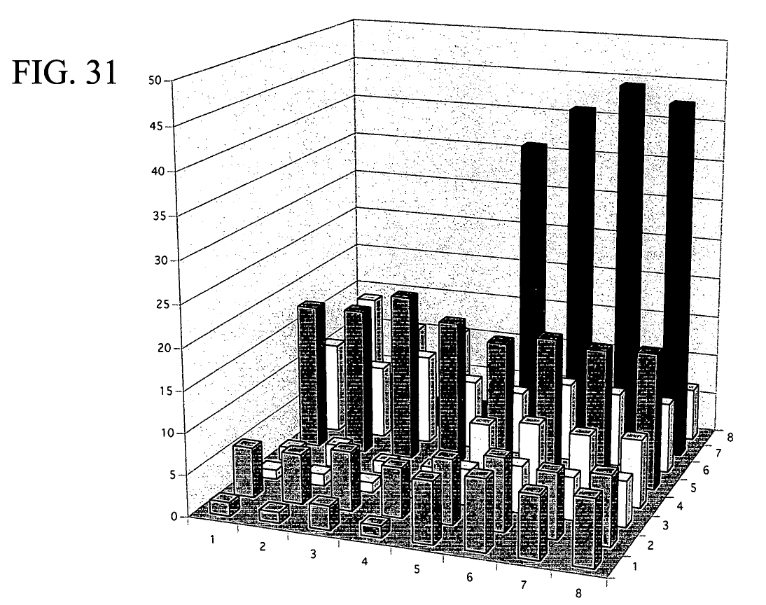

The microarrays were printed with each combination of building blocks spotted in quadruplicate. Visual inspection of a direct plot (FIG. 31) of the raw fluorescence data (from the run illustrated in FIG. 20) for one block of binding data obtained for r-phycoerythrin demonstrates that the candidate receptor environment “spots” showed reproducible binding to the test ligand. Further analysis of the r-phycoerythrin data (FIG. 20) led to only 9 out of 768 spots (1.2%) being deleted as outliers. Analysis of the r-phycoerythrin quadruplicate data for the entire array gives a mean standard deviation for each experimental quadruplicate set of 938 fluorescence units, with a mean coefficient of variation of 19.8%.

Although these values are acceptable, a more realistic comparison employed the standard deviation and coefficient of variation of the more strongly bound, more fluorescent receptors. The overall mean standard deviation unrealistically inflates the coefficient of variation for the weakly bound, less fluorescent receptors. The coefficient of variation for the 19 receptors with greater than 10,000 Fluorescent Units of bound target is 11.1%, which is well within the range required to produce meaningful binding data.

One goal of the CARA approach is the facile preparation of a significant number of candidate receptors through combinations of structurally simple building blocks. The following results establish that both the individual building blocks and combinations of building blocks have a significant, positive effect on test ligand binding.

The binding data illustrated in FIGS. 29-30 demonstrate that heterogeneous combinations of building blocks (n=2, n=3) are dramatically superior candidate receptors made from a single building block (n=1). For example, FIG. 22 illustrates both the diversity of binding observed for n=2, n=3 candidate receptors with fluorescent units ranging from 0 to ca. 40,000. These data also illustrate and the ca. 10-fold improvement in binding affinity obtained upon going from the homogeneous (n=1) to heterogeneous (n=2, n=3) receptor environments.

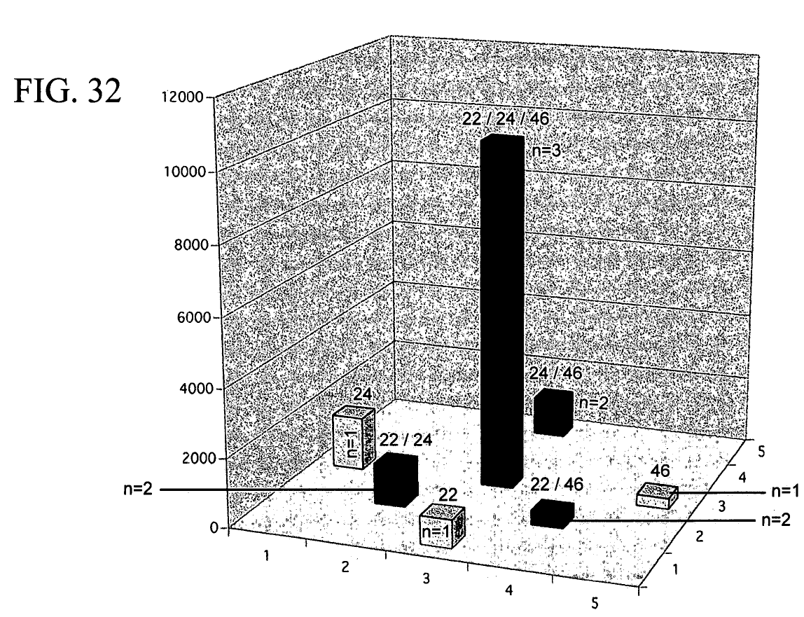

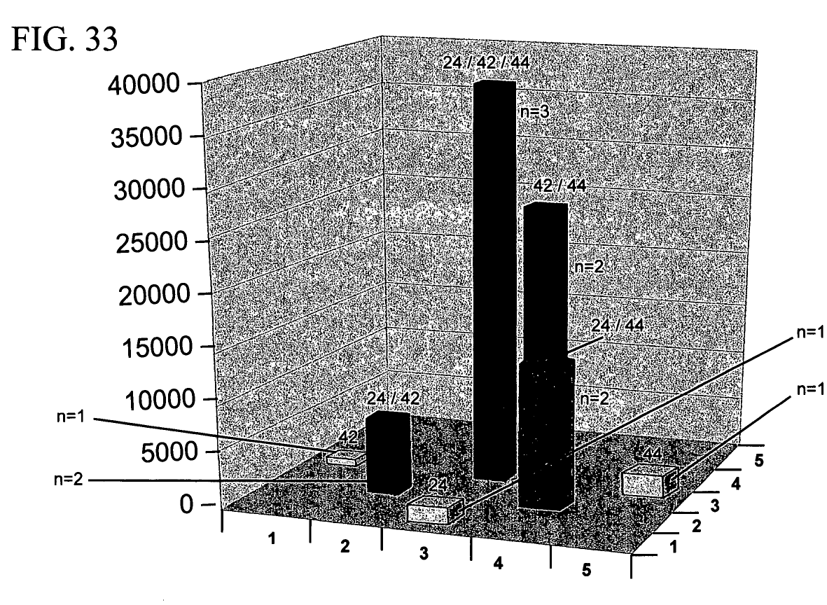

The effect of heterogeneous building blocks is most easily observed by comparing selected n=3 receptor environments candidate receptors including 1 or 2 of those building blocks (their n=2 and n=1 subsets). FIGS. 32 and 33 illustrate this comparison for two different n=3 receptor environments using the r-phycoerythrin data. In these examples, it is clear that progression from the homogeneous system (n=1) to the heterogeneous systems (n=2, n=3) produces significantly enhanced binding.

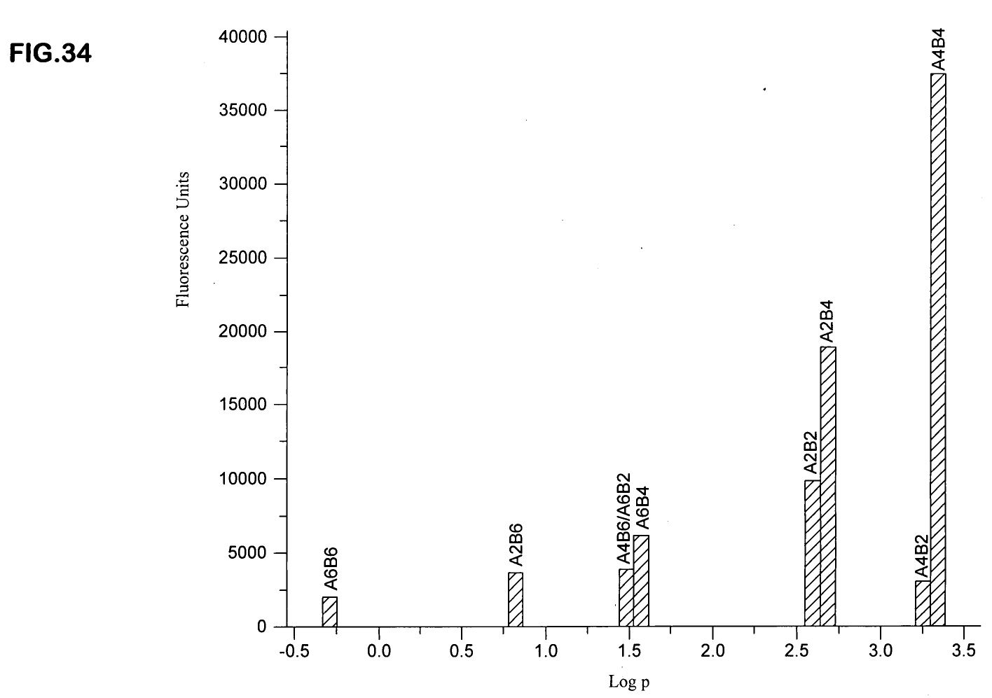

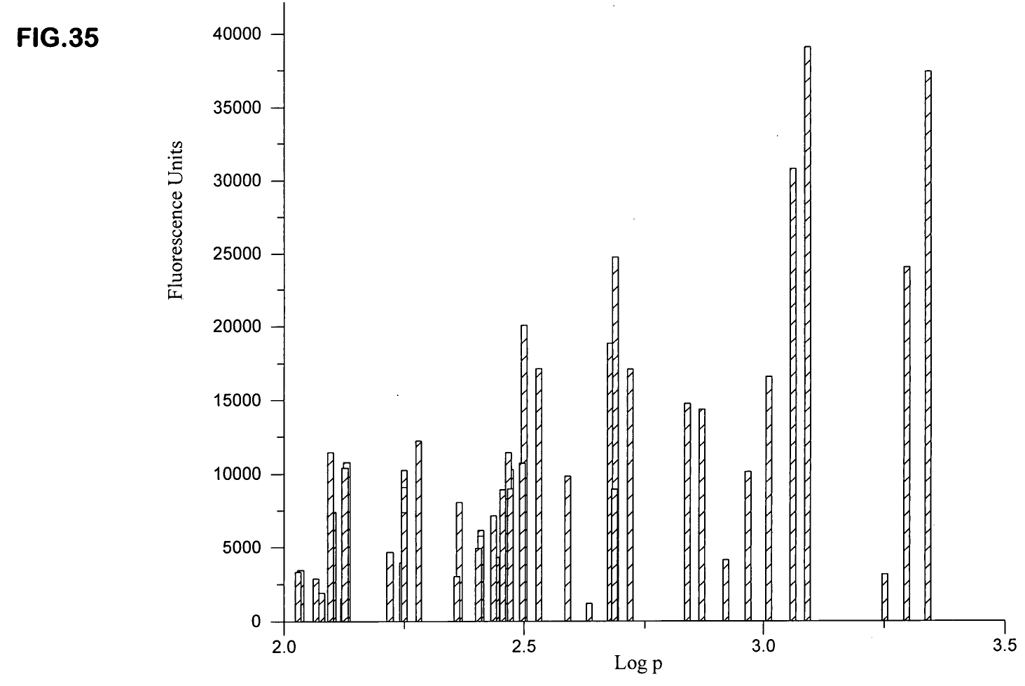

Although van der Waals interactions are an important part of molecular recognition, it is important to establish that the observed binding is not a simple case of hydrophobic/hydrophilic partitioning. That is, that the observed binding was the result of specific interactions between the individual building blocks and the target. The simplest way to evaluate the effects of hydrophobicity and hydrophilicity is to compare building block logP value with observed binding. LogP is a known and accepted measure of lipophilicity, which can be measured or calculated by known methods for each of the building blocks. FIGS. 34 and 35 establish that the observed target binding, as measured by fluorescence units, is not directly proportional to building block logP. The plots in FIGS. 34 and 35 illustrate a non-linear relationship between binding (fluorescence units) and building block logP.

One advantage of the present methods and arrays is that the ability to screen large numbers of candidate receptor environments will lead to a combination of useful target affinities and to significant target binding diversity. High target affinity is useful for specific target binding, isolation, etc. while binding diversity can provide multiplexed target detection systems. This example employed a relatively small number of building blocks to produce ca. 120 binding environments. The following analysis of the present data clearly demonstrates that even a relatively small number of binding environments can produce diverse and useful artificial receptors.

The target binding experiments performed for this study used protein concentrations including 0.1 to 10 μg/ml. Considering the BSA data as representative, it is clear that some of the receptor environments readily bound 1.0 ug/ml BSA concentrations near the saturation values for fluorescence units (see, e.g., FIG. 20). Based on these data and the formula weight of 68,000 for BSA, several of the receptor environments readily bind BSA at ca. 15 picomole/ml or 15 nanomolar concentrations. Additional experiments using lower concentrations of protein (data not shown) indicate that, even with a small selection of candidate receptor environments, femptomole/ml or picomolar detection limits have been attained.

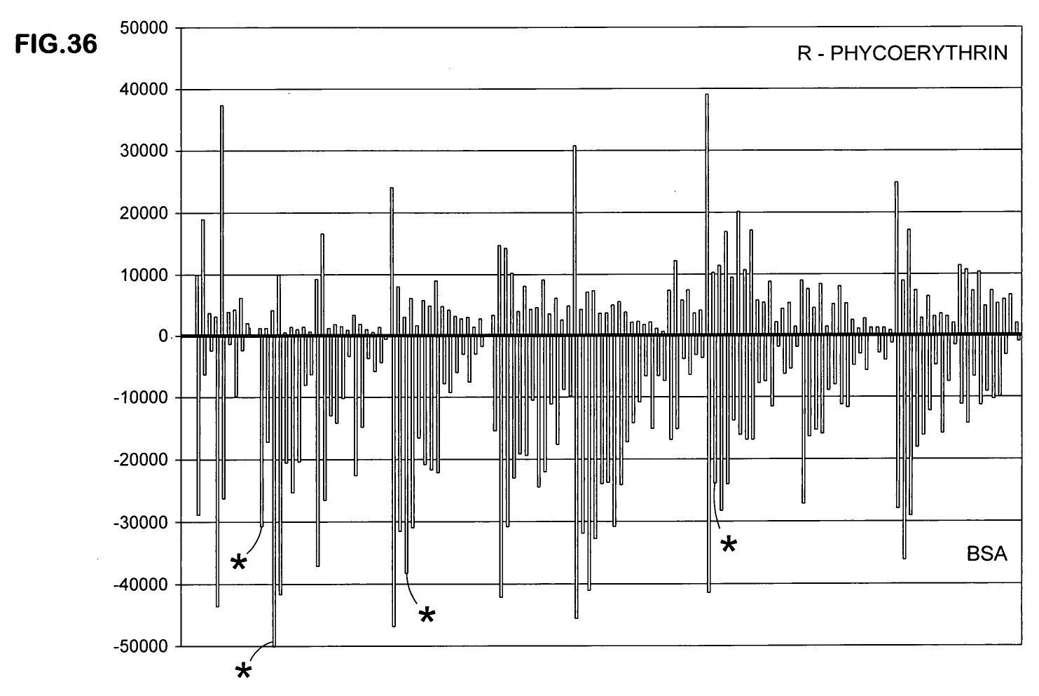

One goal of artificial receptor development is the specific recognition of a particular target. FIG. 36 compares the observed binding for r-phycoerythrin and BSA. Comparison of the overall binding pattern indicates some general similarities. However, comparison of specific features of binding for each receptor environment demonstrates that the two targets have distinctive recognition features as indicated by the (*) in FIG. 36.

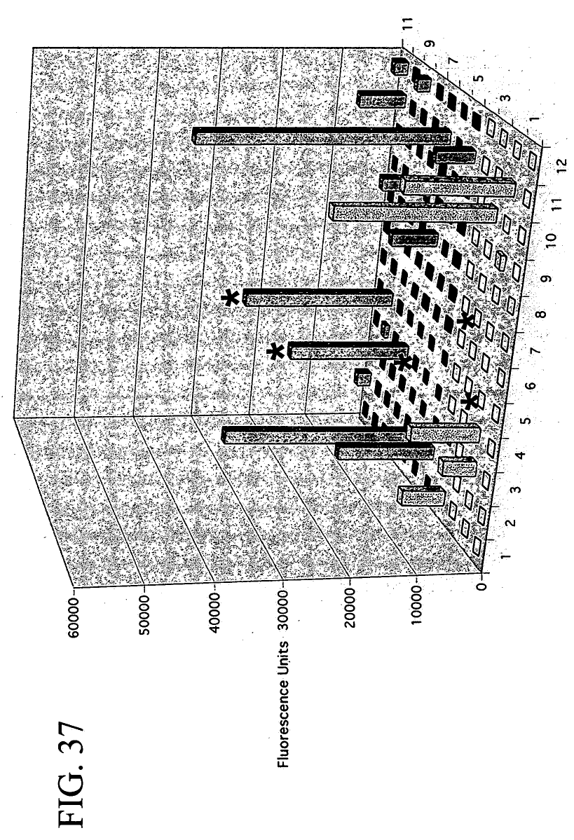

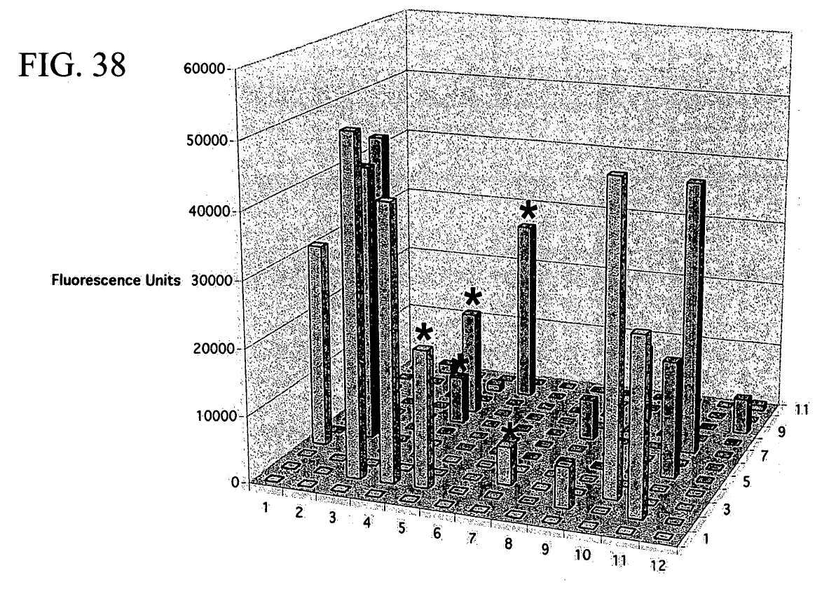

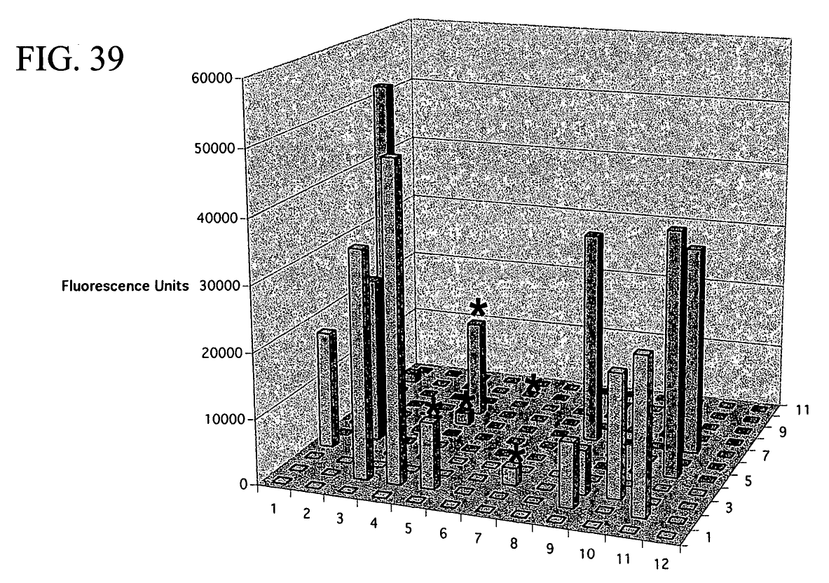

One goal of artificial receptor development is to develop receptors which can be used for the multiplexed detection of specific targets. Comparison of the r-phycoerythrin, BSA and ovalbumin data from this study (FIGS. 22, 24, and 26) were used to select representative artificial receptors for each target. FIGS. 37, 38 and 39 employ data obtained in the present example to illustrate identification of each of these three targets by their distinctive binding patterns.

### Conclusions

The optimum receptor for a particular target requires molecular recognition which is greater than the expected sum of the individual hydrophilic, hydrophobic, ionic, etc. interactions. Thus, the identification of an optimum (specific, sensitive) artificial receptor from the limited pool of candidate receptors explored in this prototype study, was not expected and not likely. Rather, the goal was to demonstrate that all of the key components of the CARA: Combinatorial Artificial Receptor Array concept could be assembled to form a functional receptor microarray. This goal has been successfully demonstrated.

This study has conclusively established that CARA microarrays can be readily prepared and that target binding to the candidate receptor environments can be used to identify artificial receptors and test ligands. In addition, these results demonstrate that there is significant binding enhancement for the building block heterogeneous (n=2, n=3, or n=4) candidate receptors when compared to their homogeneous (n=1) counterparts. When combined with the binding pattern recognition results and the demonstrated importance of both the heterogeneous receptor elements and heterogeneous building blocks, these results clearly demonstrate the significance of the CARA Candidate Artificial Receptor->Lead Artificial Receptor->Working Artificial Receptor strategy.

### Example 3

### Preparation and Evaluation of Microarrays of Candidate Artificial Receptors Including Reversibly Immobilized Building Blocks

Microarrays of candidate artificial receptors including building blocks immobilized through van der Waals interactions were made and evaluated for binding of a protein ligand. The evaluation was conducted at several temperatures, above and below a phase transition temperature for the lawn (vide infra).

Materials and Methods

Building blocks 2-2, 2-4, 2-6, 4-2, 4-4, 4-6, 6-2, 6-4, 6-6 where prepared as described in Example 1. The C12 amide was prepared using the previously described carbodiimide activation of the carboxyl followed by addition of dodecylamine. This produced a building block with a 12 carbon alkyl chain linker for reversible immobilization in the C18 lawn.

Amino lawn microarray plates (Telechem) were modified to produce the C18 lawn by reaction of stearoyl chloride (Aldrich Chemical Co.) in A) dimethylformamide/PEG 400 solution (90:10, v/v, PEG 400 is polyethylene glycol average MW 400 (Aldrich Chemical Co.) or B) methylene chloride/TEA solution (100 ml methylene chloride, 200 μl triethylamine) using the lawn modification procedures generally described in Example 2.

The C18 lawn plates where printed using the SpotBot standard procedure as described in Example 2. The building blocks were in printing solutions prepared by solution of ca. 10 mg of each building block in 300 μl of methylene chloride and 100 μl methanol. To this stock was added 900 μl of dimethylformamide and 100 μl of PEG 400. The 36 combinations of the 9 building blocks taken two at a time (N9:n2, 36 combinations) where prepared in a 384-well microwell plate which was then used in the SpotBot to print the microarray in quadruplicate. A random selection of the print positions contained only print solution. The selected microarray was incubated with a 1.0 μg/ml solution of the test ligand, cholera toxin subunit B labeled with the Alexa™ fluorophore (Molecular Probes Inc., Eugene, Oreg.), using the following variables: 1) the microarray was washed with methylene chloride, ethanol and water to create a control plate; and 2) the microarray was incubated at 4° C., 23° C., or 44° C. After incubation, the plate(s) were rinsed with water, dried and scanned (AXON 4100A). Data analysis was as described in Example 2.

Results

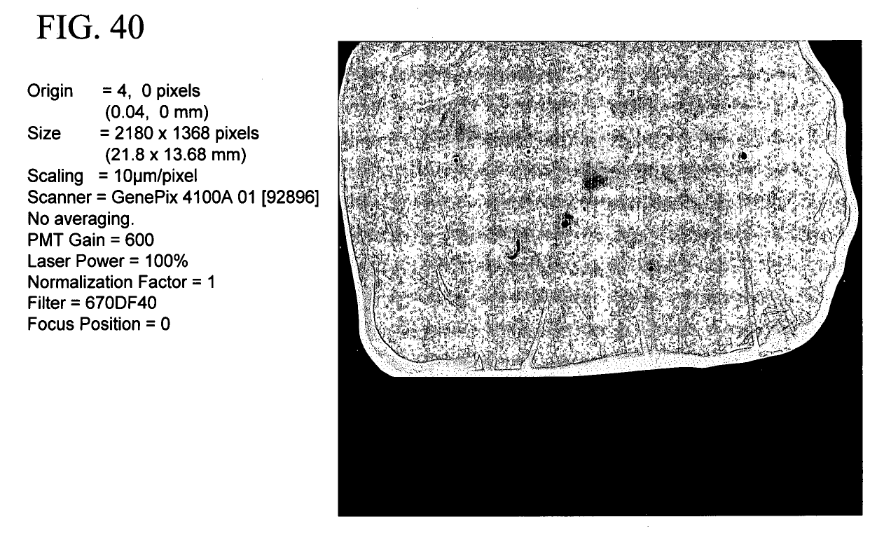

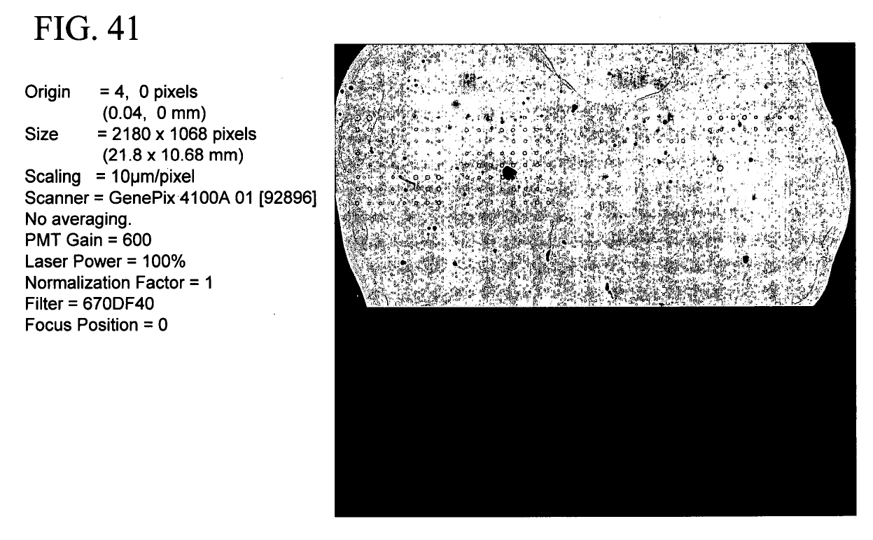

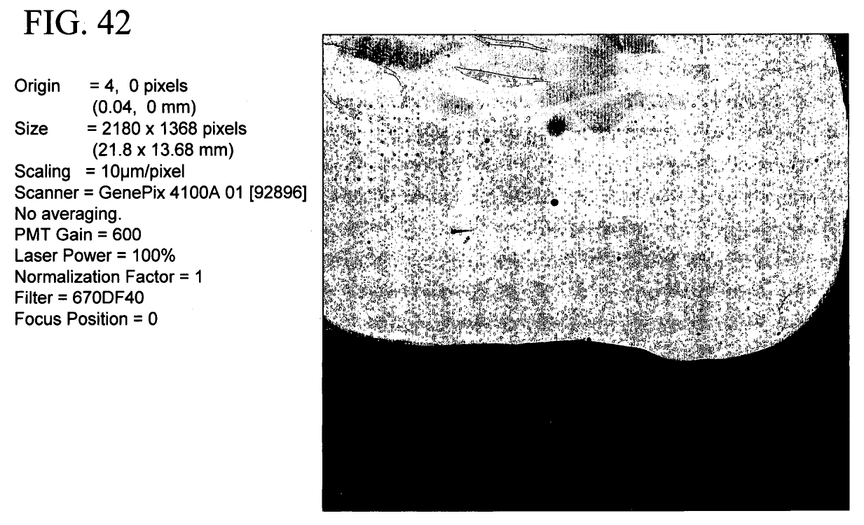

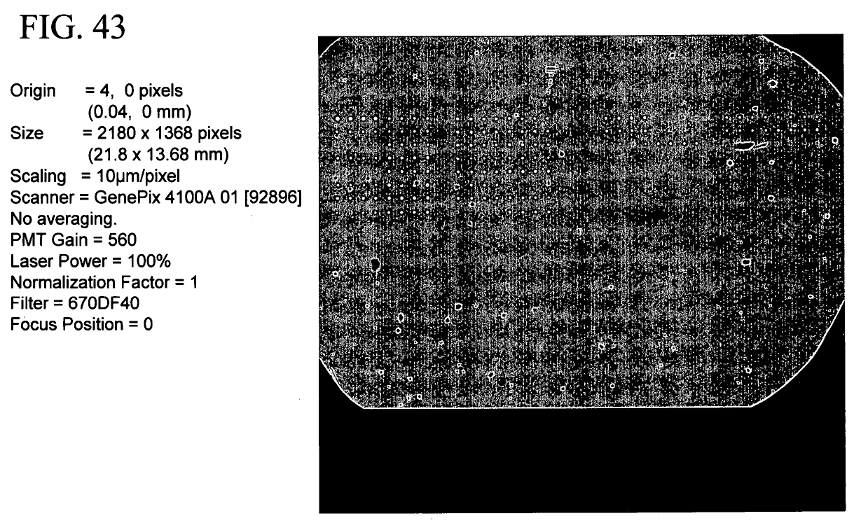

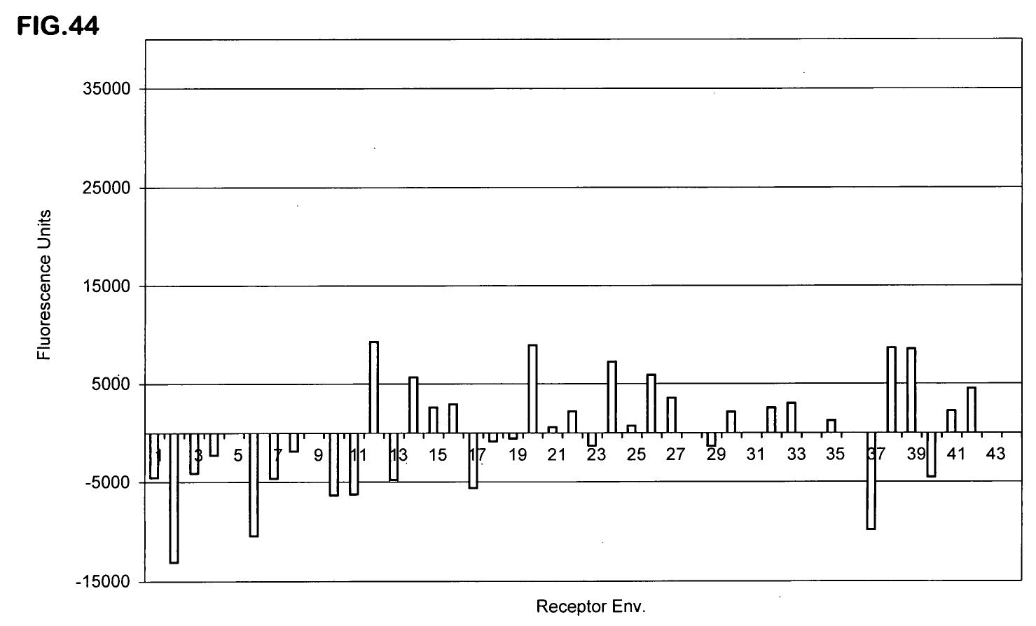

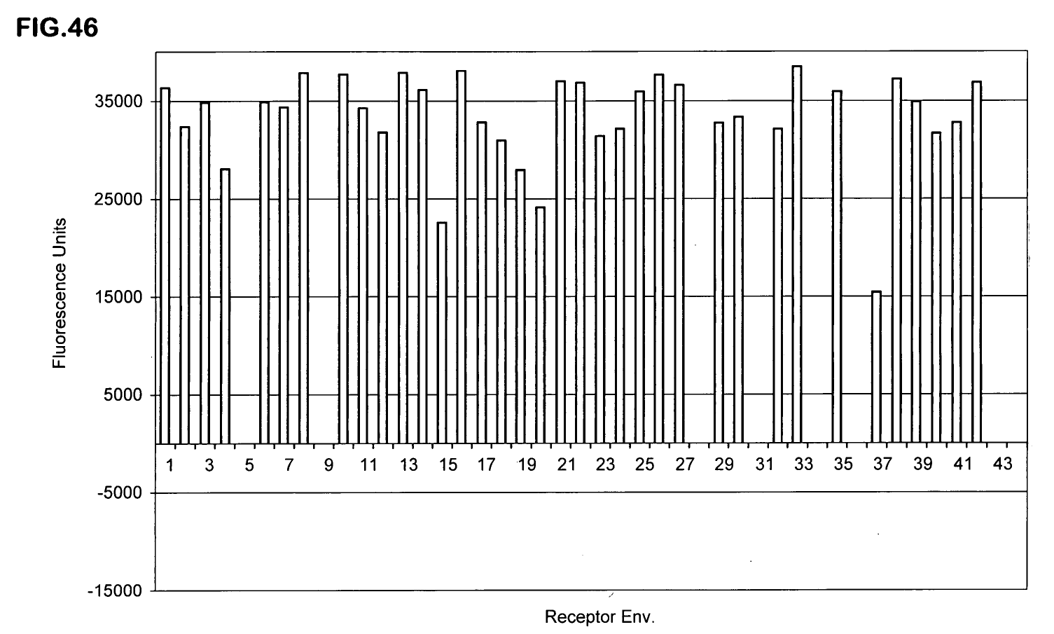

A control array from which the building blocks had been removed by washing with organic solvent did not bind cholera toxin (FIG. 40). FIGS. 41-43 illustrate fluorescence signals from arrays printed identically, but incubated with cholera toxin at 3° C., 23° C., or 43° C. Spots of fluorescence can be seen in each array, with very pronounced spots produced by incubation at 43° C. The fluorescence values for the spots in each of these three arrays are shown in FIGS. 44-46. Fluorescence signal generally increases with temperature, with many nearly equally large signals observed after incubation at 43° C. Linear increases with temperature can reflect expected improvements in binding with temperature. Nonlinear increases reflect rearrangement of the building blocks on the surface to achieve improved binding, which occurred above the phase transition for the lipid surface (vide infra).

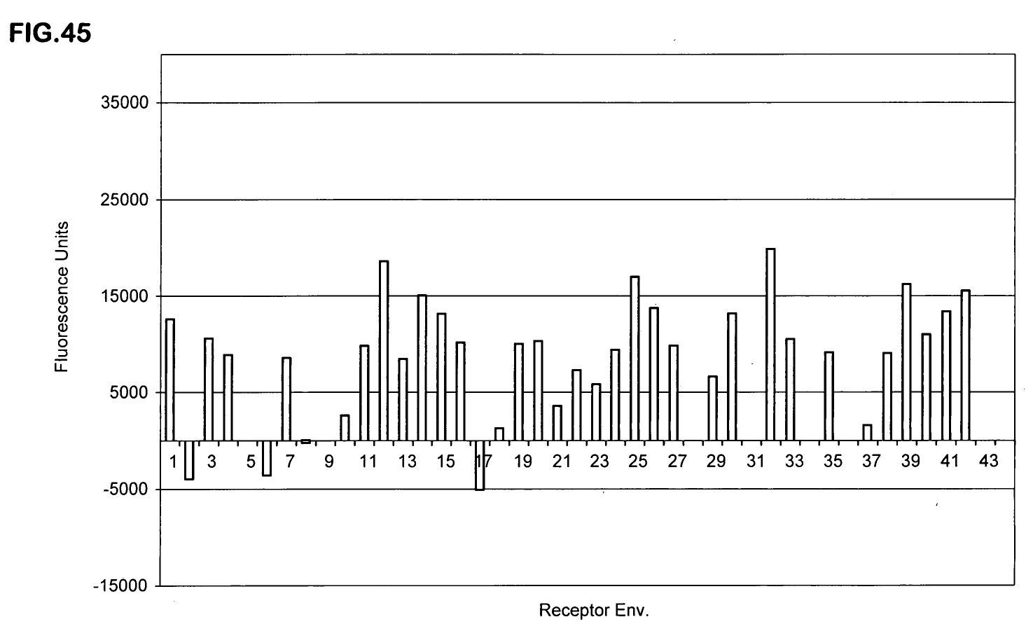

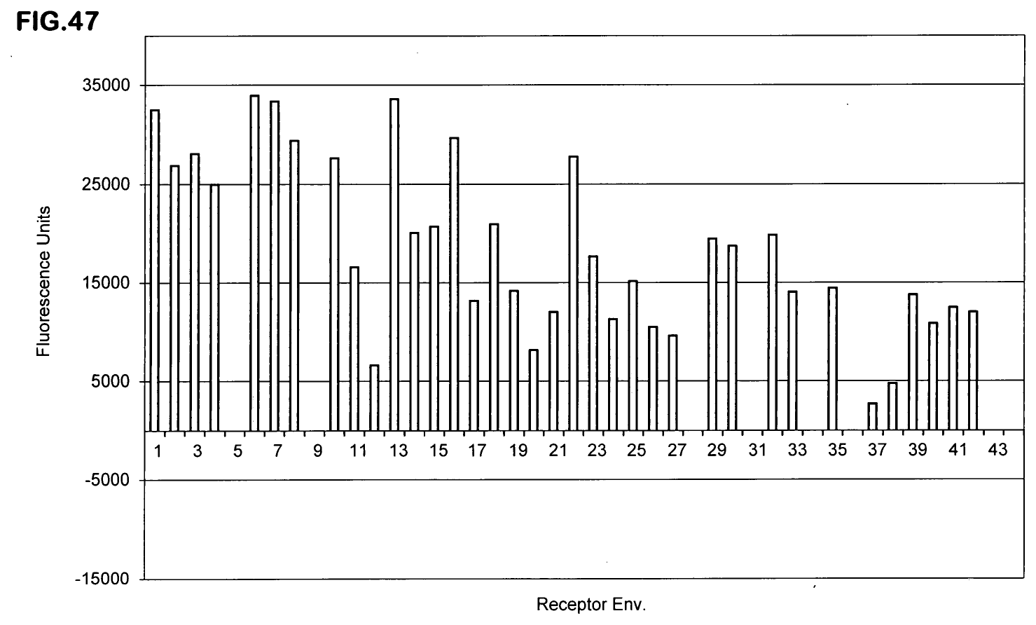

FIG. 47 can be compared to FIG. 45. The fluorescence signals plotted in FIG. 45 resulted from binding to reversibly immobilized building blocks on a support at 23° C. The fluorescence signals plotted in FIG. 47 resulted from binding to covalently immobilized building blocks on a support at 23° C. These figures compare the same combinations of building blocks in the same relative positions, but immobilized in two different ways.

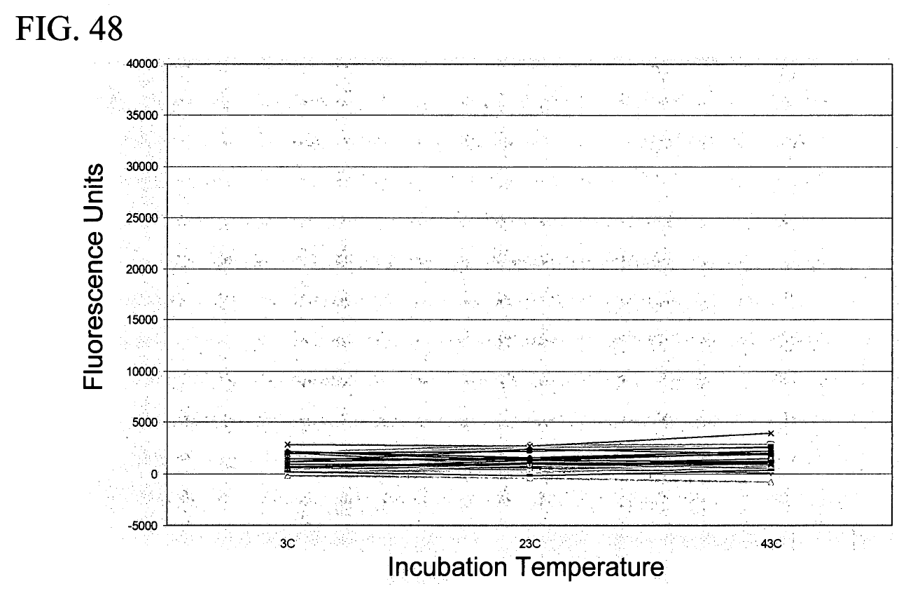

The binding to covalently immobilized building blocks was also evaluated at 3° C., 23° C., or 43° C. FIG. 48 illustrates the changes in fluorescence signal from individual combinations of covalently immobilized building blocks at 3° C., 23° C., or 43° C. Binding increased modestly with temperature. The mean increase in binding was 1.3-fold. A plot of the fluorescence signal for each of the covalently immobilized artificial receptors at 23° C. against its signal at 43° C. (not shown) yields a linear correlation with a correlation coefficient of 0.75. This linear correlation indicates that the mean 1.3-fold increase in binding is a thermodynamic effect and not optimization of binding.

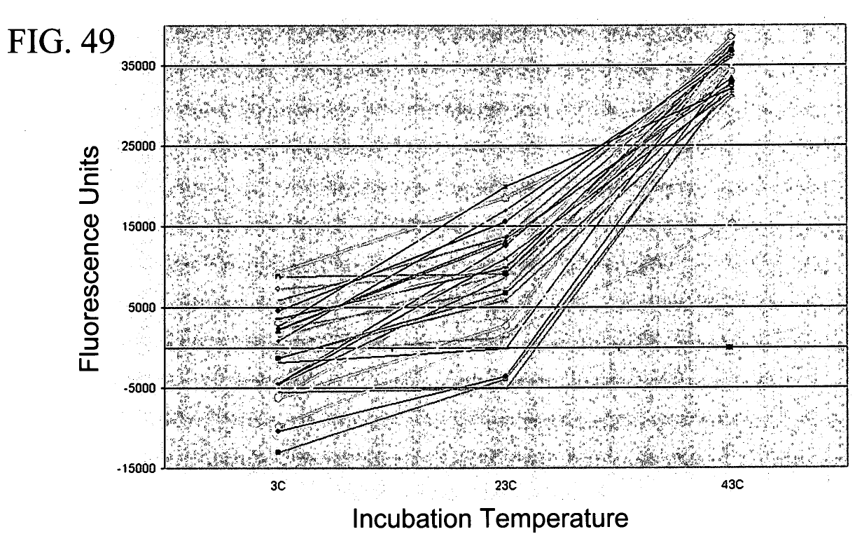

FIG. 49 illustrates the changes in fluorescence signal from individual combinations of reversibly immobilized building blocks at 3° C., 23° C., or 43° C. This graph illustrates that at least one combination of building blocks (candidate artificial receptor) exhibited a signal that remained constant as temperature increased. At least one candidate artificial receptor exhibited an approximately linear increase in signal as temperature increased. Such a linear increase indicates normal temperature effects on binding. The candidate artificial receptor with the lowest binding signal at 3° C. became one of the best binders at 43° C. This indicates that rearrangement of the building blocks of this receptor above the phase transition for the lawn, which increases the building blocks' mobility, produced increased binding. Other receptors characterized by greater changes in binding between 23° C. and 43° C. (compared to between 3° C. and 23° C.) also underwent dynamic affinity optimization.

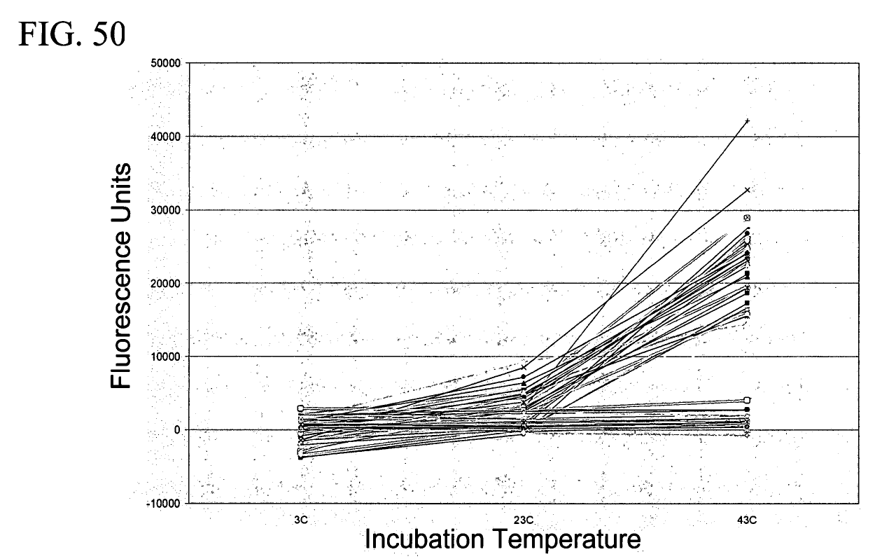

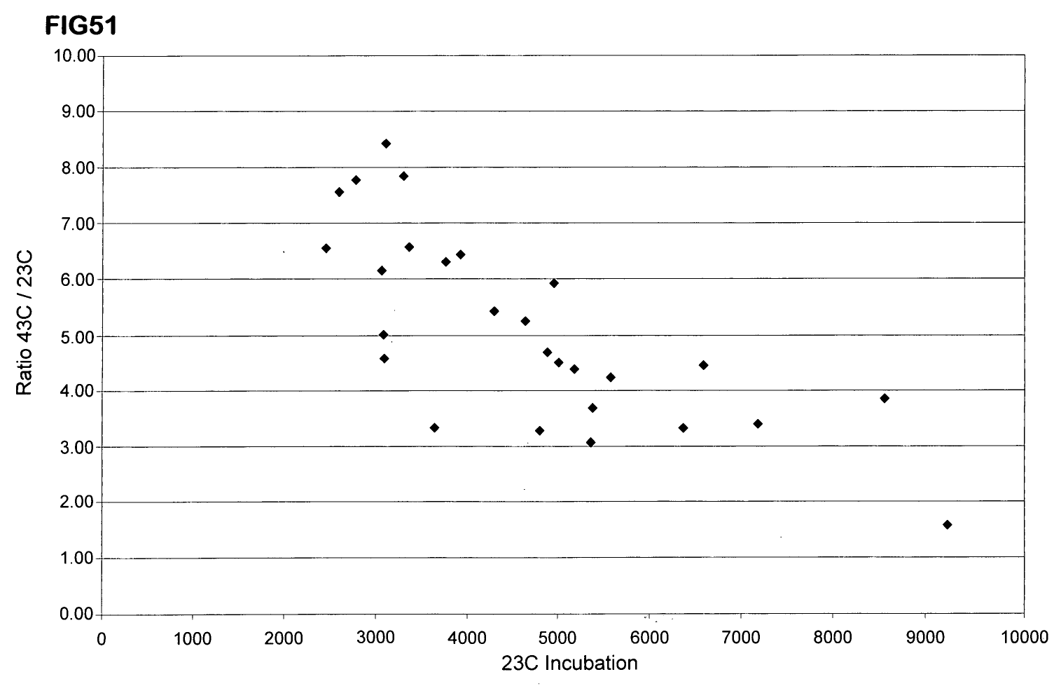

FIG. 50 illustrates the data presented in FIG. 48 (lines marked A) and the data presented in FIG. 49 (lines marked B). The increases in binding observed with the reversibly immobilized building blocks are significantly greater than the increases observed with covalently bound building blocks. Binding to reversibly immobilized building blocks increased from 23° C. and 43° C. by a median value of 6.1-fold and a mean value of 24-fold. This confirms that movement of the reversibly immobilized building blocks within the receptors increased binding (i.e., the receptor underwent dynamic affinity optimization).

A plot of the fluorescence signal for each of the reversibly immobilized artificial receptors at 23° C. against its signal at 43° C. (not shown) yields no correlation (correlation coefficient of 0.004). A plot of the fluorescence signal for each of the reversibly immobilized artificial receptors at 43° C. against the signal for the corresponding covalently immobilized receptor (not shown) also yields no correlation (correlation coefficient 0.004).

This lack of correlation provides further evidence that movement of the reversibly immobilized building blocks within the receptors increased binding.

FIG. 51 illustrates a graph of the fluorescence signal at 43° C. divided by the signal at 23° C. against the fluorescence signal obtained from binding at 23° C. for the artificial receptors with reversibly immobilized receptors. This comparison indicates that the binding enhancement is independent of the initial affinity of the receptor for the test ligand.

Table 1 identifies the reversibly immobilized building blocks making up each of the artificial receptors, lists the fluorescence signal (binding strength) at 43° C. and 23° C., and the ratios of the observed binding at these two temperatures. These data illustrate that each artificial receptor reflects a unique attribute for each combination of building blocks relative to the role of each individual building block.

TABLE 1

| Building Blocks | | | Ratio of |
|---|---|---|---|
| Making Up | | | Signals, |
| Receptor | Signal at 43° C. | Signal at 23° C. | 43° C./23° C. |
| 22 24 | 24136 | 4611 | 5.23 |
| 22 26 | 16660 | 43 | 387.44 |
| 22 42 | 17287 | −167 | −103.51 |
| 22 44 | 16726 | 275 | 60.82 |
| 22 46 | 25016 | 3903 | 6.41 |
| 22 62 | 13990 | 3068 | 4.56 |
| 22 64 | 15294 | 3062 | 4.99 |
| 22 66 | 11980 | 3627 | 3.30 |
| 24 26 | 22688 | 1291 | 17.57 |
| 24 42 | 26808 | −662 | −40.50 |
| 24 44 | 23154 | 904 | 25.61 |
| 24 46 | 42197 | 2814 | 15.00 |
| 24 62 | 19374 | 2567 | 7.55 |
| 24 64 | 27599 | 262 | 105.34 |
| 24 66 | 16238 | 5334 | 3.04 |
| 26 42 | 22282 | 4974 | 4.48 |
| 26 44 | 26240 | 530 | 49.51 |
| 26 46 | 23144 | 4273 | 5.42 |
| 26 62 | 29022 | 4920 | 5.90 |
| 26 64 | 23416 | 5551 | 4.22 |
| 26 66 | 19553 | 5353 | 3.65 |
| 42 44 | 29093 | 6555 | 4.44 |
| 42 46 | 18637 | 3039 | 6.13 |
| 42 62 | 22643 | 4853 | 4.67 |
| 42 64 | 20836 | 6343 | 3.28 |
| 42 66 | 14391 | 9220 | 1.56 |
| 44 46 | 25600 | 3266 | 7.84 |
| 44 62 | 15544 | 4771 | 3.26 |
| 44 64 | 25842 | 3073 | 8.41 |
| 44 66 | 22471 | 5142 | 4.37 |
| 46 62 | 32764 | 8522 | 3.84 |
| 46 64 | 21901 | 3343 | 6.55 |
| 46 66 | 23516 | 3742 | 6.28 |
| 62 64 | 24069 | 7149 | 3.37 |
| 62 66 | 15831 | 2424 | 6.53 |
| 64 66 | 21310 | 2746 | 7.76 |

### Conclusions

This experiment demonstrated that an array including reversibly immobilized building blocks binds a protein substrate, like an array with covalently immobilized building blocks. The binding increased nonlinearly as temperature increased, indicating that movement of the building blocks increased binding. Many of the candidate artificial receptors demonstrated improved binding upon mobilization of the building blocks.

### Example 4

### The Oligosaccharide Portion of GM1 Competes with Artificial Receptors for Binding to Cholera Toxin

Microarrays of candidate artificial receptors were made and evaluated for binding of cholera toxin. The arrays were also evaluated for disrupting that binding. Disrupting of binding employed a compound that binds to cholera toxin, the oligosaccharide moiety from GM1 (GM1 OS). The results obtained demonstrate that a ligand of a protein specifically disrupted binding of the protein to the microarray.

Materials and Methods

Building blocks were synthesized and activated as described in Example 1. The building blocks employed in this example were TyrA1B1 [1-1], TyrA2B2, TyrA2B4, TyrA2B6, TyrA2B8, TyrA3B3, TyrA3B5, TyrA3B7, TyrA4B2, TyrA4B4, TyrA4B6, TyrA4B8, TyrA5B3, TyrA5B5, TyrA5B7, TyrA6B2, TyrA6B4, TyrA6B6, TyrA6B8, TyrA7B3, TyrA7B5, TyrA7B7, TyrA8B2, TyrA8B4, TyrA8B6, and TyrA8B8. The abbreviation for the building block including a linker, a tyrosine framework, and recognition elements AxBy is TyrAxBy.

Microarrays for the evaluation of the 171 n=2 candidate receptor environments were prepared as follows by modifications of known methods. An “n=2” receptor environment includes two different building blocks. Briefly: Amine modified (amine “lawn”; SuperAmine Microarray plates) microarray plates were purchased from Telechem Inc., Sunnyvale, Calif. These plates were manufactured specifically for microarray preparation and had a nominal amine load of 2-4 amines per square nm according to the manufacturer. The microarrays were prepared using a pin microarray spotter instrument from Telechem Inc. (SpotBot™ Arrayer) typically with 200 μm diameter spotting pins from Telechem Inc. Stealth Micro Spotting Pins, SMP6) and 400-420 μm spot spacing.

The 19 building blocks were activated in aqueous dimethylformamide (DMF) olution as described above. For preparing the 384-well feed plate, the activated building lock solutions were diluted 10-fold with a solution of DMF/H₂O/PEG400 (90/10/10, v/v/v; PEG400 is polyethylene glycol nominal 400 FW, Aldrich Chemical Co., Milwaukee, Wis.). These stock solutions were aliquotted (10 μl per aliquot) into the wells of a 384-well microwell plate (Telechem Inc.). Control spots included the building block [1-1]. The plate was covered with aluminum foil and placed on the bed of a rotary shaker for 15 minutes at 1,000 RPM. This master plate was stored covered with aluminum foil at −20° C. when not in use.

For preparing the 384-well SpotBot™ plate, a well-to-well transfer (e.g. A-1 to A-1, A-2 to A-2, etc.) from the feed plate to a second 384-well plate was performed using a 4 μl transfer pipette. This plate was stored tightly covered with aluminum foil at −20° C. when not in use. The SpotBot™ was used to prepare up to 13 microarray plates per run using the 4 μl microwell plate. The SpotBot™ was programmed to spot from each microwell in quadruplicate. The wash station on the SpotBot™ used a wash solution of EtOH/H₂O (20/80, v/v). This wash solution was adjusted to pH 4 with 1 M HCl and used to rinse the microarrays on completion of the SpotBot™ printing run. The plates were given a final rinse with deionized (DI) water, dried using a stream of compressed air, and stored at room temperature. The microarrays were further modified by reacting the remaining amines with acetic anhydride to form an acetamide lawn in place of the amine lawn.

The test ligand employed in these experiments was cholera toxin labeled with the Alexa™ fluorophore (Molecular Probes Inc., Eugene, Oreg.). The candidate disruptor employed in these experiments was GM1 OS (GM1 oligosaccharide), a known ligand for cholera toxin.

Microarray incubation and analysis was conducted as follows: For control incubations with the microarrays, solutions (e.g. 500 μl) of the cholera toxin in PBS-T (PBS with 20 μl/L of Tween-20) at a concentrations of 1.7 pmol/ml (0.1 μg/ml) was placed onto the surface of a microarray and allowed to react for 30 minutes. For disruptor incubations with the microarrays, solutions (e.g. 500 μl) of the cholera toxin (1.7 pmol/ml, 0.1 μg/ml) and the desired concentration of GM1 OS in PBS-T (PBS with 20 μl/L of Tween-20) was placed onto the surface of a microarray and allowed to react for 30 minutes. GM1 OS was added at 0.34 and at 5.1 μM in separate experiments. After either of these incubations, the microarray was rinsed with PBS-T and DI water and dried using a stream of compressed air.

The incubated microarray was scanned using an Axon Model 4200A Fluorescence Microarray Scanner (Axon Instruments, Union City, Calif.). The Axon scanner and its associated software produce a false color 16-bit image of the fluorescence intensity of the plate. This 16-bit data is integrated using the Axon software to give a Fluorescence Units value (range 0-65,536) for each spot on the microarray. This data is then exported into an Excel file (Microsoft) for further analysis including mean, standard deviation and coefficient of variation calculations.

Table 2 identifies the building blocks in each of the first 150 receptor environments.

TABLE 2

| | Building Blocks |
|---|---|
| 1 | 22 24 |
| 2 | 22 28 |
| 3 | 22 42 |
| 4 | 22 46 |
| 5 | 22 55 |
| 6 | 22 64 |
| 7 | 22 68 |
| 8 | 22 82 |
| 9 | 22 86 |
| 10 | 24 26 |
| 11 | 24 33 |
| 12 | 24 44 |
| 13 | 26 77 |
| 14 | 26 84 |
| 15 | 26 88 |
| 16 | 28 42 |
| 17 | 22 26 |
| 18 | 22 33 |
| 19 | 22 44 |
| 20 | 22 48 |
| 21 | 22 62 |
| 22 | 22 66 |
| 23 | 22 77 |
| 24 | 22 84 |
| 25 | 22 88 |
| 26 | 24 28 |
| 27 | 24 42 |
| 28 | 26 82 |
| 29 | 26 85 |
| 30 | 28 33 |
| 31 | 28 44 |
| 32 | 28 46 |
| 33 | 28 55 |
| 34 | 28 64 |
| 35 | 28 68 |
| 36 | 28 82 |
| 37 | 28 86 |
| 38 | 33 42 |
| 39 | 33 46 |
| 40 | 42 88 |
| 41 | 44 48 |
| 42 | 44 62 |
| 43 | 44 66 |
| 44 | 44 77 |
| 45 | 44 84 |
| 46 | 44 88 |
| 47 | 46 55 |
| 48 | 28 48 |
| 49 | 28 62 |
| 50 | 28 66 |
| 51 | 28 77 |
| 52 | 28 84 |
| 53 | 28 88 |
| 54 | 33 44 |
| 55 | 44 46 |
| 56 | 44 55 |
| 57 | 44 64 |
| 58 | 44 68 |
| 59 | 44 82 |
| 60 | 44 86 |
| 61 | 46 48 |
| 62 | 46 62 |
| 63 | 24 46 |
| 64 | 24 55 |
| 65 | 24 64 |
| 66 | 24 68 |
| 67 | 24 82 |
| 68 | 24 86 |
| 69 | 26 28 |
| 70 | 26 42 |
| 71 | 26 46 |
| 72 | 26 55 |
| 73 | 26 64 |
| 74 | 26 68 |
| 75 | 33 48 |
| 76 | 33 63 |
| 77 | 33 66 |
| 78 | 33 77 |
| 79 | 24 48 |
| 80 | 24 62 |
| 81 | 24 66 |
| 82 | 24 77 |
| 83 | 24 84 |
| 84 | 24 88 |
| 85 | 26 33 |
| 86 | 26 44 |
| 87 | 26 48 |
| 88 | 26 62 |
| 89 | 26 66 |
| 90 | 33 55 |
| 91 | 33 64 |
| 92 | 33 68 |
| 93 | 33 82 |
| 94 | 33 84 |
| 95 | 33 88 |
| 96 | 42 46 |
| 97 | 42 55 |
| 98 | 42 64 |
| 99 | 42 68 |
| 100 | 42 82 |
| 101 | 42 86 |
| 102 | 46 88 |
| 103 | 48 62 |
| 104 | 48 66 |
| 105 | 46 77 |
| 106 | 48 84 |
| 107 | 48 88 |
| 108 | 55 64 |
| 109 | 55 68 |
| 110 | 33 86 |
| 111 | 42 44 |
| 112 | 42 48 |
| 113 | 42 62 |
| 114 | 42 66 |
| 115 | 42 77 |
| 116 | 42 84 |
| 117 | 48 55 |
| 118 | 48 64 |
| 119 | 48 68 |
| 120 | 48 82 |
| 121 | 48 86 |
| 122 | 55 62 |
| 123 | 55 66 |
| 124 | 55 77 |
| 125 | 46 64 |
| 126 | 46 68 |
| 127 | 46 82 |
| 128 | 46 86 |
| 129 | 62 77 |
| 130 | 62 84 |
| 131 | 62 88 |
| 132 | 64 68 |
| 133 | 64 82 |
| 134 | 64 86 |
| 135 | 66 68 |
| 136 | 66 82 |
| 137 | 66 86 |
| 138 | 68 77 |
| 139 | 68 84 |
| 140 | 68 88 |
| 141 | 46 66 |
| 142 | 46 77 |
| 143 | 46 84 |
| 144 | 62 82 |
| 145 | 62 86 |
| 146 | 64 66 |
| 147 | 64 77 |
| 148 | 64 84 |
| 149 | 64 88 |
| 150 | 66 77 |

Results
Low Concentration of GM1 OS

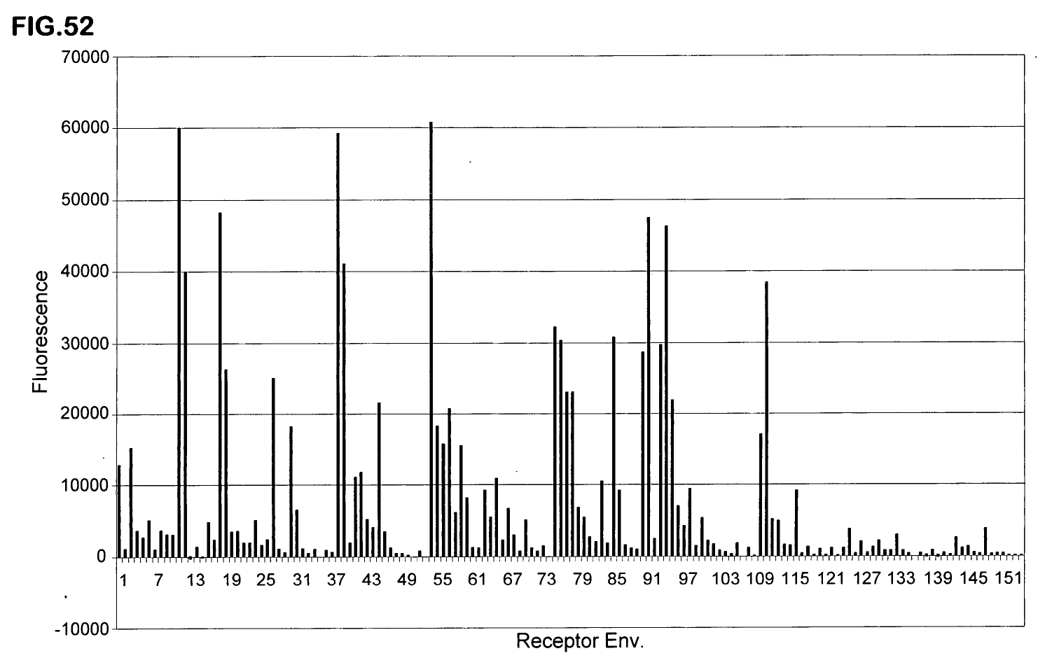

FIG. 52 illustrates binding of cholera toxin to the microarray of candidate artificial receptors followed by washing with buffer produced fluorescence signals. These fluorescence signals demonstrate that the cholera toxin bound strongly to certain receptor environments, weakly to others, and undetectably to some. Comparison to experiments including those reported in Example 2 indicates that cholera toxin binding was reproducible from array to array and from month to month.

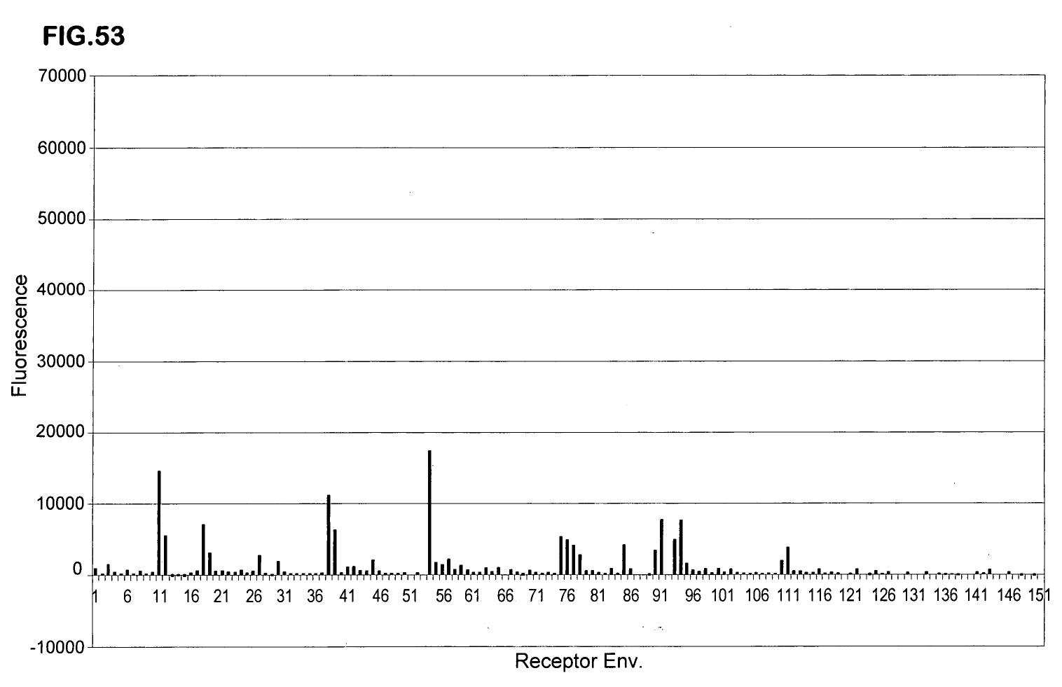

Binding of cholera toxin was also conducted with competition from GM1 OS (0.34 μM). FIG. 53 illustrates the fluorescence signals due to cholera toxin binding that were detected after this competition. Notably, many of the signals illustrated in FIG. 53 are significantly smaller than the corresponding signals recorded in FIG. 52. The small signals observed in FIG. 53 represent less cholera toxin bound to the array. GM1 OS significantly disrupted binding of cholera toxin to many of the receptor environments.

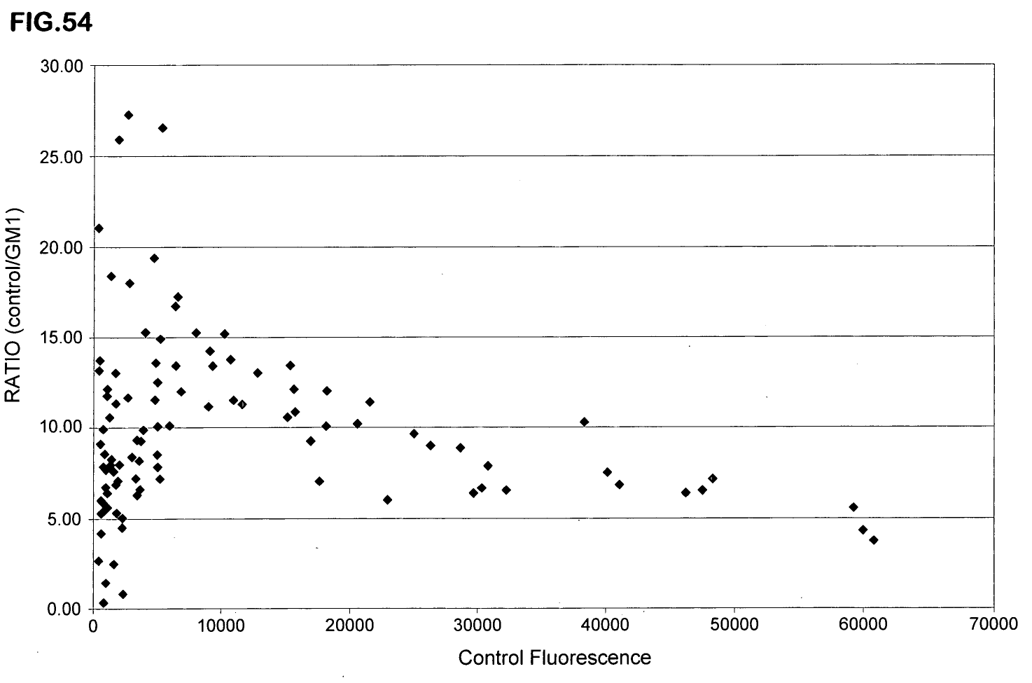

The disruption in cholera toxin binding caused by GM1 OS can be visualized as the ratio of the amount bound in the absence of GM1 OS to the amount bound in competition with GM1 OS. This ratio is illustrated in FIG. 54. The larger the ratio, the less cholera toxin remained bound to the artificial receptor after competition with GM1 OS. The ratio can be as large as about 30. The ratios are independent of the quantity bound in the control.

High Concentration of GM1 OS

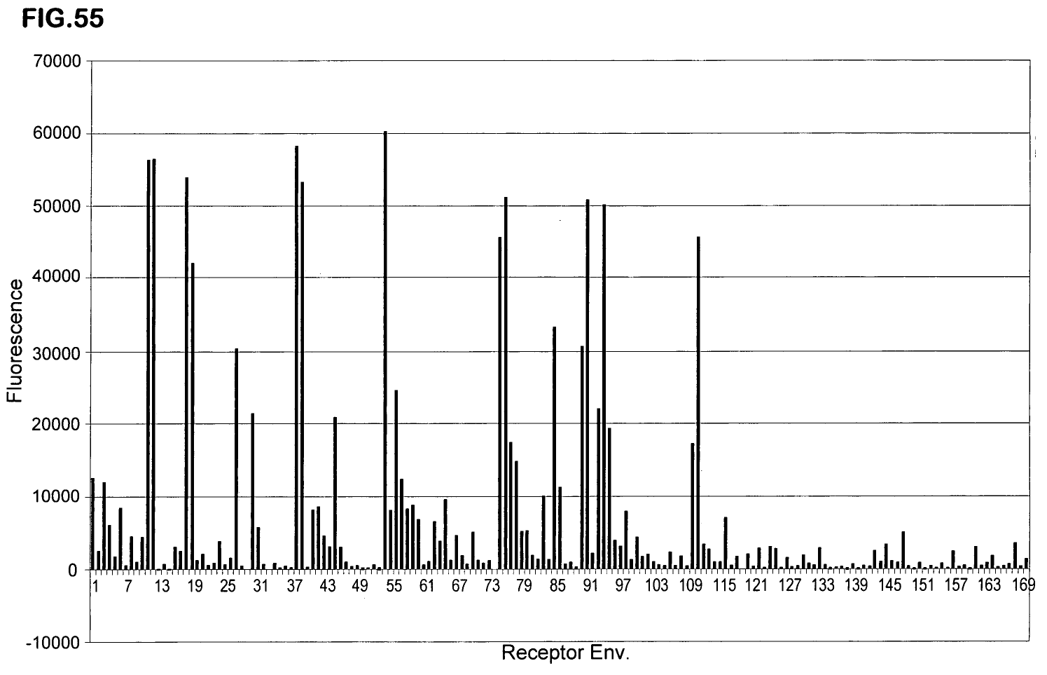

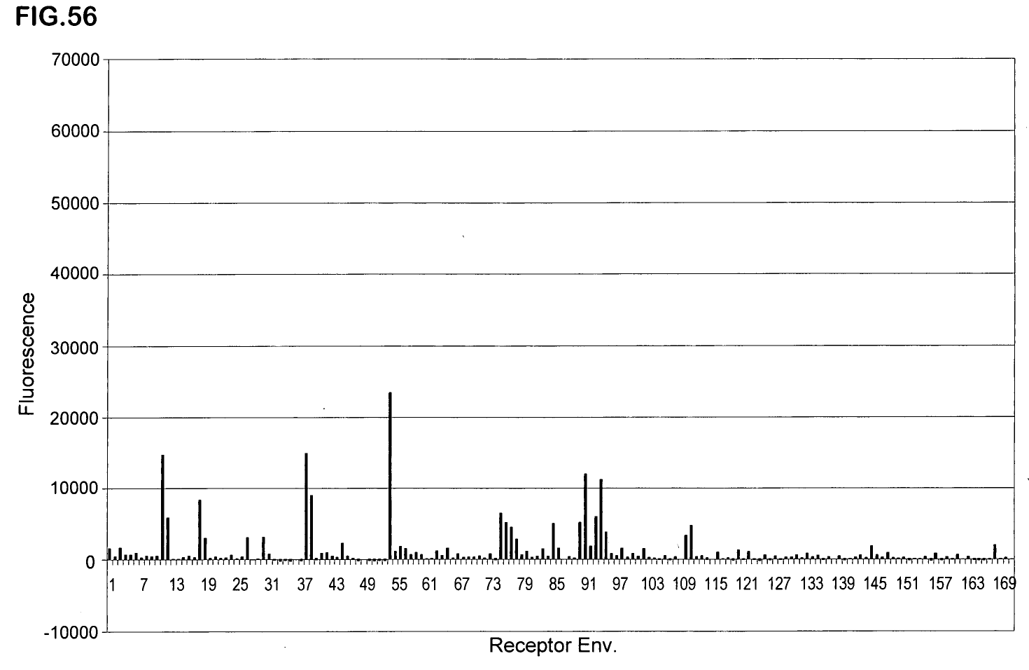

Binding of cholera toxin to the microarray of candidate artificial receptors followed by washing with buffer produced fluorescence signals illustrated in FIG. 55. As before, cholera toxin was reproducible and it bound strongly to certain receptor environments, weakly to others, and undetectably to some. FIG. 56 illustrates the fluorescence signals detected due to cholera toxin binding that were detected upon competition with GM1 OS at 5.1 μM. Again, GM1 OS significantly disrupted binding of cholera toxin to many of the receptor environments.

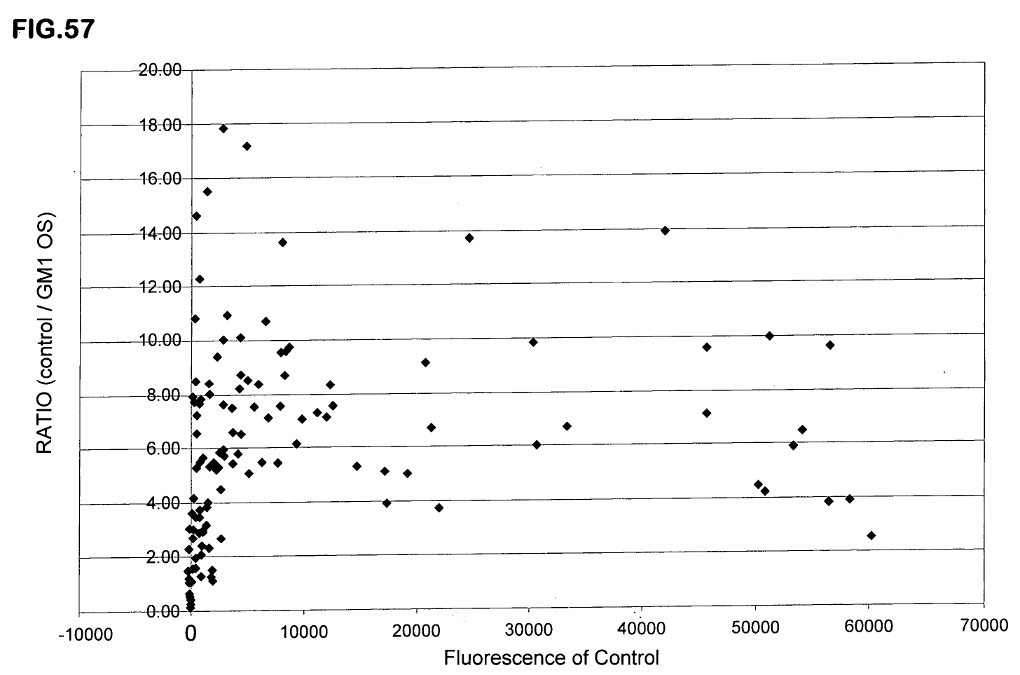

This disruption is presented as the ratio of the amount bound in the absence of GM1 OS to the amount bound after contacting with GM1 OS in FIG. 57. The ratios range up to about 18 and are independent of the quantity bound in the control.

### Conclusions

This experiment demonstrated that binding of a test ligand to an artificial receptor of the present invention can be diminished (e.g., competed) by a candidate disrupter molecule. In this case the test ligand was the protein cholera toxin and the candidate disruptor was a compound known to bind to cholera toxin, GM1 OS. The degree to which binding of the test ligand was disrupted was independent of the degree to which the test ligand bound to the artificial receptor.

### Example 5

### GM1 Competes with Artificial Receptors for Binding to Cholera Toxin

Microarrays of candidate artificial receptors were made and evaluated for binding of cholera toxin. The arrays were also evaluated for disrupting that binding. Disrupting of binding employed a compound that binds to cholera toxin, the liposaccharide GM1. The results obtained demonstrate that a ligand of a protein specifically disrupts binding of the protein to the microarray.

Materials and Methods

Building blocks were synthesized and activated as described in Example 1. The building blocks employed in this example were TyrA1B1 [1-1], TyrA2B2, TyrA2B4, TyrA2B6, TyrA4B2, TyrA4B4, TyrA4B6, TyrA6B2, TyrA6B4, and TyrA6B6 in groups of 4 building blocks per artificial receptor. The abbreviation for the building block including a linker, a tyrosine framework, and recognition elements AxBy is TyrAxBy.

Microarrays for the evaluation of the 126 n=4 candidate receptor environments were prepared as described above for Example 4. The test ligand employed in these experiments was cholera toxin labeled with the Alexa™ fluorophore (Molecular Probes Inc., Eugene, Oreg.). Cholera toxin was employed at 5.3 nM in both the control and the competition experiments. The candidate disruptor employed in these experiments was GM1, a known ligand for cholera toxin, which competed at concentrations of 0.042, 0.42, and 8.4 μM. Microarray incubation and analysis was conducted as described for Example 4.

Table 3 identifies the building blocks in each receptor environment.

TABLE 3

| | Building Blocks |
|---|---|
| 1 | 22 24 26 42 |
| 2 | 22 24 26 44 |
| 3 | 22 24 26 46 |
| 4 | 22 24 26 61 |
| 5 | 22 24 26 64 |
| 6 | 22 24 26 66 |
| 7 | 22 24 42 44 |
| 8 | 22 24 42 46 |
| 9 | 22 24 42 62 |
| 10 | 22 24 42 46 |
| 11 | 22 24 42 66 |
| 12 | 22 24 44 46 |
| 13 | 22 24 44 62 |
| 14 | 22 24 44 64 |
| 15 | 22 24 44 66 |
| 16 | 22 24 46 62 |
| 17 | 22 24 46 64 |
| 18 | 22 24 46 66 |
| 19 | 22 24 62 64 |
| 20 | 22 24 62 66 |
| 21 | 22 24 64 66 |
| 22 | 22 26 42 44 |
| 23 | 22 26 42 46 |
| 24 | 22 26 42 62 |
| 25 | 22 26 42 64 |
| 26 | 22 26 42 66 |
| 27 | 22 26 44 46 |
| 28 | 22 26 44 62 |
| 29 | 22 26 44 64 |
| 30 | 22 26 44 66 |
| 31 | 22 26 46 62 |
| 32 | 22 26 46 64 |
| 33 | 22 26 46 66 |
| 34 | 22 26 62 64 |
| 35 | 22 26 62 66 |
| 36 | 22 26 64 66 |
| 37 | 22 42 44 46 |
| 38 | 22 42 44 62 |
| 39 | 22 42 44 64 |
| 40 | 22 42 44 66 |
| 41 | 22 42 46 62 |
| 42 | 22 42 46 64 |
| 43 | 22 42 46 66 |
| 44 | 22 42 62 64 |
| 45 | 22 42 62 66 |
| 46 | 22 42 64 66 |
| 47 | 22 44 46 62 |
| 48 | 22 44 46 64 |
| 49 | 22 44 46 66 |
| 50 | 22 44 62 64 |
| 51 | 22 44 62 66 |
| 52 | 22 44 64 66 |
| 53 | 22 46 62 64 |
| 54 | 22 46 62 66 |
| 55 | 22 46 64 66 |
| 56 | 22 62 64 66 |
| 57 | 24 26 42 44 |
| 58 | 24 26 42 46 |
| 59 | 24 26 42 62 |
| 60 | 24 26 42 64 |
| 61 | 24 26 42 66 |
| 62 | 24 26 44 46 |
| 63 | 24 26 44 62 |
| 64 | 24 26 44 64 |
| 65 | 24 26 44 66 |
| 66 | 24 26 46 62 |
| 67 | 24 26 46 64 |
| 68 | 24 26 46 66 |
| 69 | 24 26 62 64 |
| 70 | 24 26 62 66 |
| 71 | 24 26 64 66 |
| 72 | 24 42 44 46 |
| 73 | 24 42 44 62 |
| 74 | 24 42 44 64 |
| 75 | 24 42 44 66 |
| 76 | 24 42 46 62 |
| 77 | 24 42 46 64 |
| 78 | 24 42 46 66 |
| 79 | 24 42 62 64 |
| 80 | 24 42 62 66 |
| 81 | 24 42 64 66 |
| 82 | 24 44 46 62 |
| 83 | 24 44 46 64 |
| 84 | 24 44 46 66 |
| 85 | 24 44 62 64 |
| 86 | 24 44 62 66 |
| 87 | 24 44 64 66 |
| 88 | 24 46 62 64 |
| 89 | 24 46 62 66 |
| 90 | 24 46 64 66 |
| 91 | 24 62 64 66 |
| 92 | 26 42 44 46 |
| 93 | 26 42 44 62 |
| 94 | 26 42 44 64 |
| 95 | 26 42 44 66 |
| 96 | 26 42 46 62 |
| 97 | 26 42 46 64 |
| 98 | 26 42 46 66 |
| 99 | 26 42 62 64 |
| 100 | 26 42 62 66 |
| 101 | 26 42 64 66 |
| 102 | 26 44 46 62 |
| 103 | 26 44 46 64 |
| 104 | 26 44 46 66 |
| 105 | 26 44 62 64 |
| 106 | 26 44 62 66 |
| 107 | 26 44 64 66 |
| 108 | 26 46 62 64 |
| 109 | 26 46 62 66 |
| 110 | 26 46 64 66 |
| 111 | 26 62 64 66 |
| 112 | 42 44 46 62 |
| 113 | 42 44 46 64 |
| 114 | 42 44 46 66 |
| 115 | 42 44 62 64 |
| 116 | 42 44 62 66 |
| 117 | 42 44 64 66 |
| 118 | 42 46 62 64 |
| 119 | 42 46 62 66 |
| 120 | 42 46 64 66 |
| 121 | 42 62 64 66 |
| 122 | 44 46 62 64 |
| 123 | 44 46 62 66 |
| 124 | 44 46 64 66 |
| 125 | 44 62 64 66 |
| 126 | 46 62 64 66 |

Results

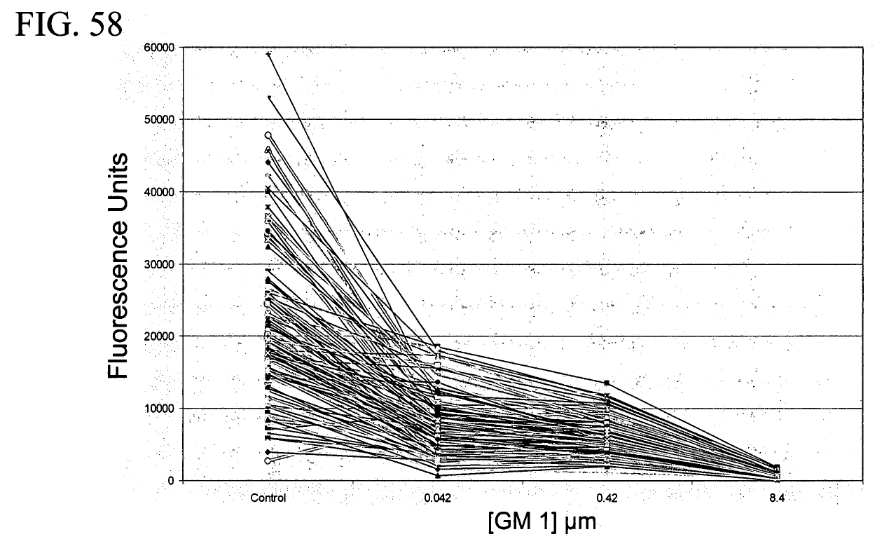

FIG. 58 illustrates the fluorescence signals produced by binding of cholera toxin to the microarray of candidate artificial receptors alone and in competition with each of the three concentrations of GM1. The magnitude of the fluorescence signal decreases steadily with increasing concentration of GM1. The amount of decrease is not quantitatively identical for all of the receptors, but each receptor experienced decreased binding of cholera toxin. These decreases indicate that GM1 competed with the artificial receptor for binding to the cholera toxin.

The decreases show a pattern of relative competition for the binding site on cholera toxin. This can be demonstrated through graphs of fluorescence signal obtained at a particular concentration of GM1 against fluorescence signal in the absence of GM1 (not shown). Certain of the receptors appear at similar relative positions on these plots as concentration of GM1 increases.

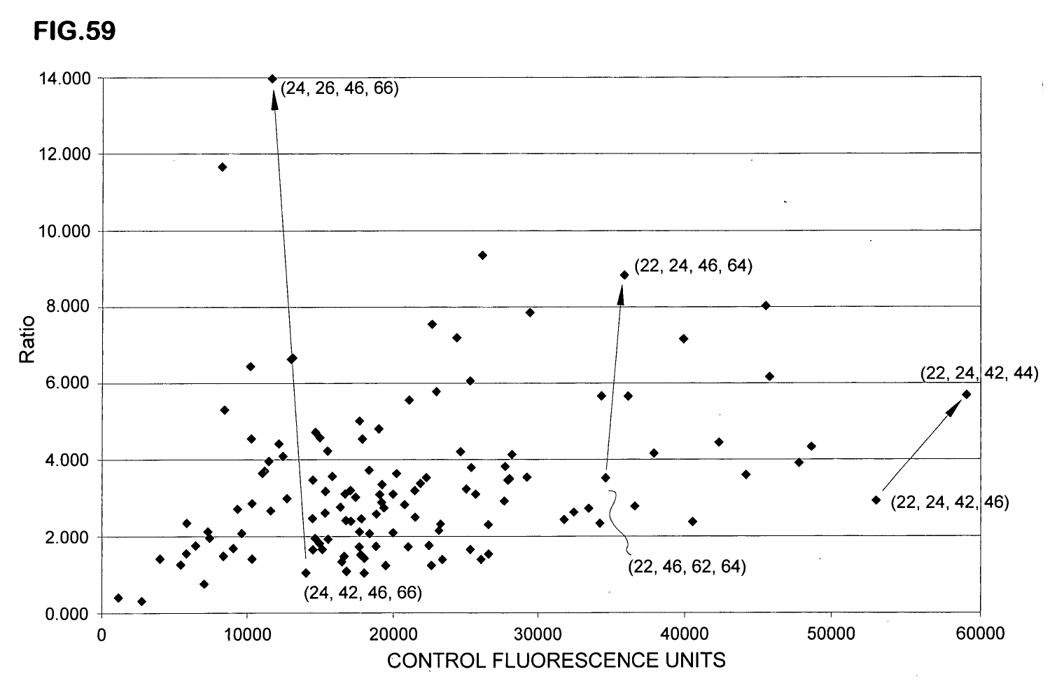

The disruption in cholera toxin binding caused by GM1 can be visualized as the ratio of the amount bound in the absence of GM1 OS to the amount bound upon competition with GM1. This ratio is illustrated in FIG. 59. The larger the ratio, the more cholera toxin remained bound to the artificial receptor upon competition with GM1. The ratio can be as large as about 14. The ratios are independent of the quantity bound in the control.

Interestingly, in several instances minor changes in structure to the artificial receptor caused significant changes in the ratio. For example, the artificial receptor including building blocks 24, 26, 46, and 66 differs from that including 24, 42, 46, and 66 by only substitution of a single building block. (xy indicates building block TyrAxBy.) The substitution of building block 42 for 26 increased binding in the presence of GM1 by about 14-fold.

By way of further example, the artificial receptor including building blocks 22, 24, 46, and 64 differs from that including 22, 46, 62, and 64 by only substitution of a single building block. The substitution of building block 24 for 62 increased binding in the presence of GM1 by about 3-fold.

Even substitution of a single recognition element affected binding. The artificial receptor including building blocks 22, 24, 42, and 44 differs from that including 22, 24, 42, and 46 by only substitution of a single recognition element. The substitution of building block 44 for 46 (a change of recognition element B6 to B4) increased binding in the presence of GM1 by about 3-fold.

### Conclusions

This experiment demonstrated that binding of a test ligand to an artificial receptor of the present invention can be diminished (e.g., competed) by a candidate disruptor molecule.

In this case the test ligand was the protein cholera toxin and the candidate disruptor was a compound known to bind to cholera toxin, GM1. Minor changes in structure of the building blocks making up the artificial receptor caused significant changes in the competition.

### Example 6

### GM1 Employed as a Building Block Alters Binding of Cholera Toxin to the Present Artificial Receptors

Microarrays of candidate artificial receptors were made, GM1 was bound to the arrays, and they were evaluated for binding of cholera toxin. The results obtained demonstrate that adding GM1 as a building block in an array of artificial receptors can increase binding to certain of the receptors.

Materials and Methods

Building blocks were synthesized and activated as described in Example 1. The building blocks employed in this example were those described in Example 4. Microarrays for the evaluation of the 171 n=2 candidate receptor environments were prepared as described above for Example 4. The test ligand employed in these experiments was cholera toxin labeled with the Alexa™ fluorophore (Molecular Probes Inc., Eugene, Oreg.). Cholera toxin was employed at 0.01 ug/ml (0.17 pM) or 0.1 ug/ml (1.7 pM) in both the control and the competition experiments. GM1 was employed as a test ligand for the artificial receptors and became a building block for receptors used to bind cholera toxin. The arrays were contacted with GM1 at either 100 μg/ml, 10 μg/ml, or 1 μg/ml as described above for cholera toxin and then rinsed with deionized water. The arrays were then contacted with cholera toxin under the conditions described above. Microarray analysis was conducted as described for Example 4. Table 2 identifies the building blocks in each receptor environment.

Results

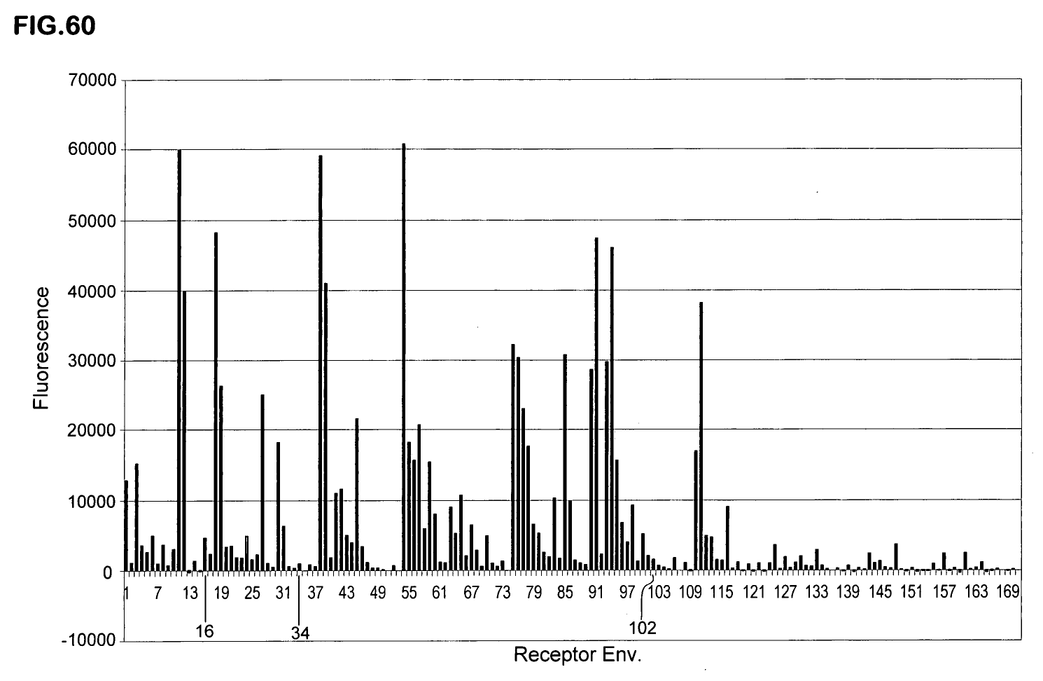

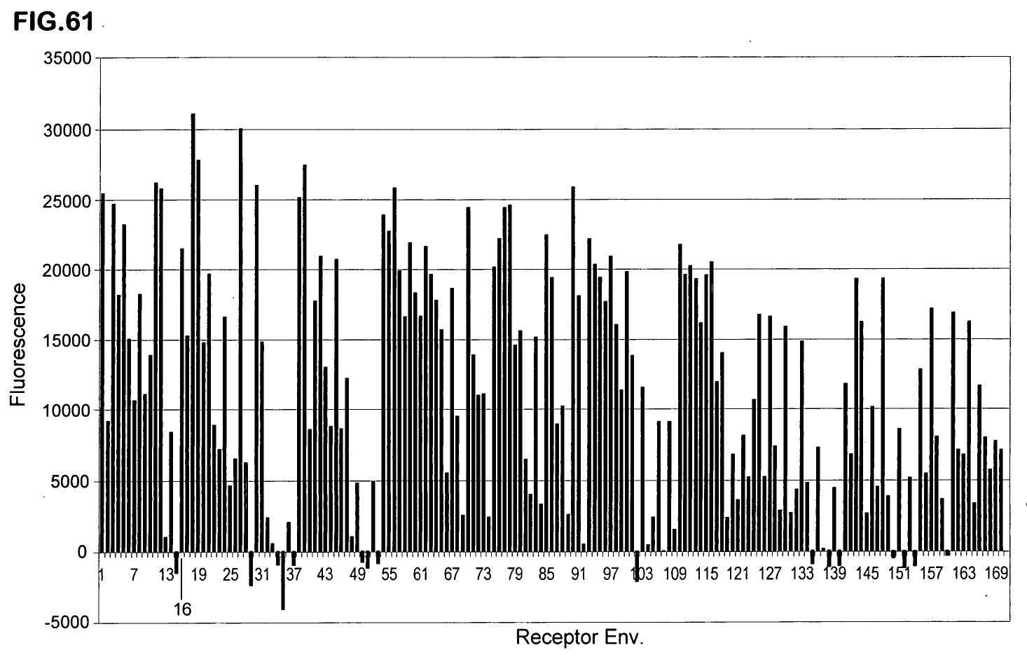

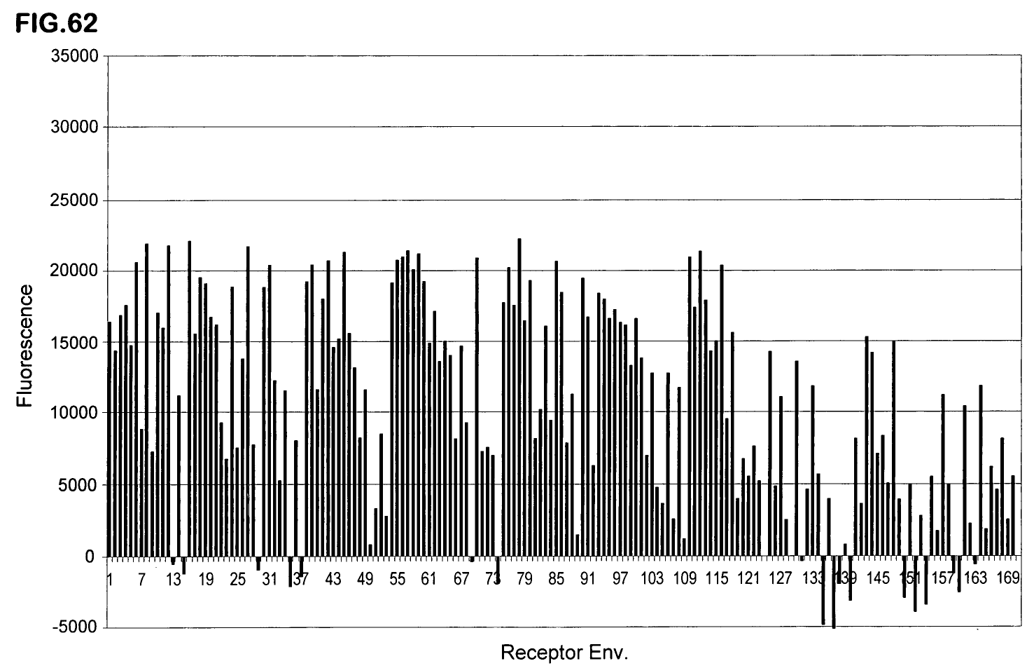

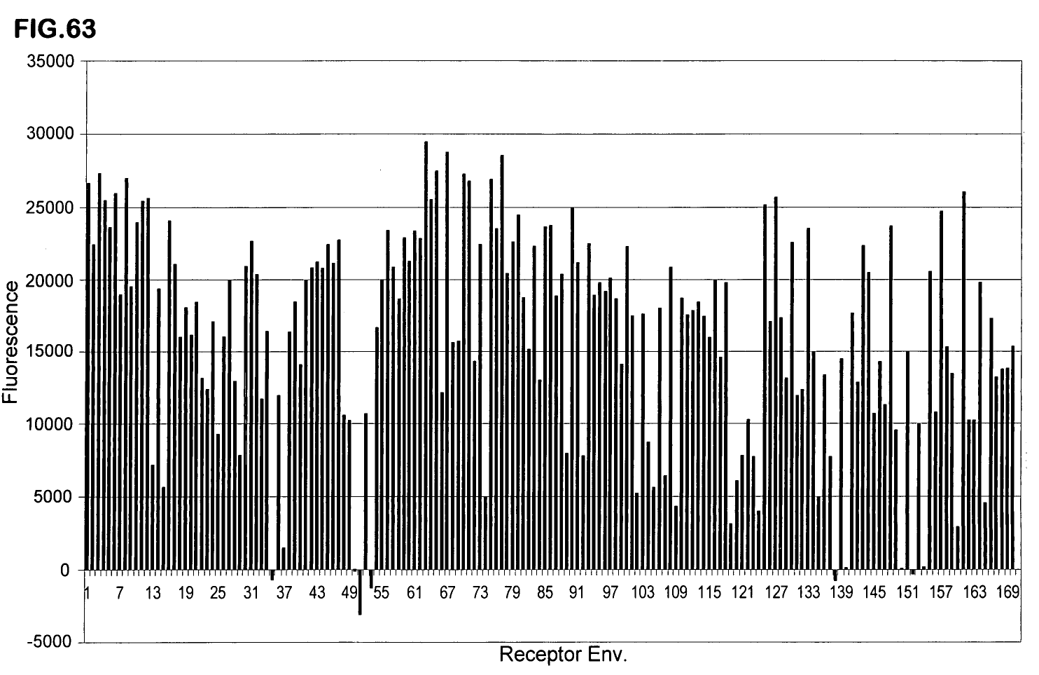

FIG. 60 illustrates the fluorescence signals produced by binding of cholera toxin to the microarray of candidate artificial receptors without pretreatment with GM1. Binding of GM1 to the microarray of candidate artificial receptors followed by binding of cholera toxin produced fluorescence signals illustrated in FIGS. 61, 62, and 63 (100 μg/ml, 10 μg/ml, and 1 μg/ml GM1, respectively).

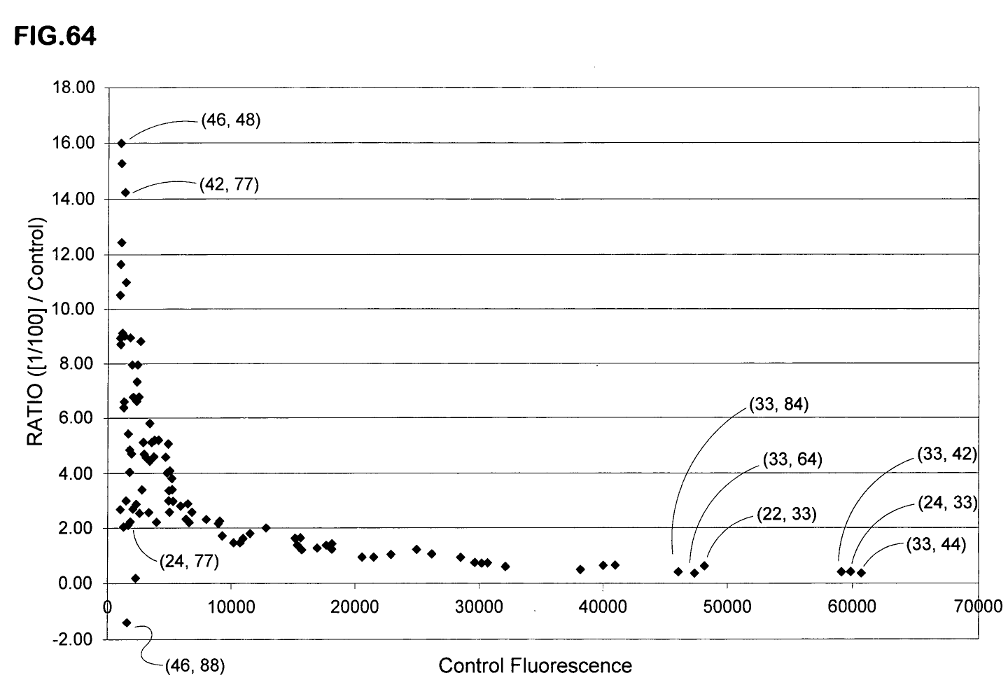

The enhancement of cholera toxin binding caused by pretreatment with GM1 can be visualized as the ratio of the amount bound in the presence of GM1 to the amount bound in the absence of GM1. This ratio is illustrated in FIG. 64 for 1 μg/ml GM1. The larger the ratio, the more cholera toxin bound to the artificial receptor after pretreatment with GM1. The ratio can be as large as about 16.

In several instances minor changes in structure to the artificial receptor caused significant changes in the ratio. For example, the artificial receptor including building blocks 46 and 48 differs from that including 46 and 88 by only substitution of a single recognition element on a single building block. (xy indicates building block TyrAxBy.) The substitution of building block 48 for 88 (a change of recognition element A8 to A4) increased the ratio representing increased binding the presence of GM1 building block from about 0.5 to about 16. Similarly, the artificial receptor including building blocks 42 and 77 differs from that including 24 and 77 by only substitution of a single building block. The substitution of building block 42 for 24 increased the ratio representing increased binding the presence of GM1 building block from about 2 to about 14.

Interestingly, several building blocks that exhibited high levels of binding of cholera toxin (signals of 45,000 to 65,000 fluorescence units) and that include the building block 33 were not strongly affected by the presence of GM1 as a building block.

### Conclusions

This experiment demonstrated that binding of GM1 to an artificial receptor of the present invention can significantly increase binding by cholera toxin. Minor changes in structure of the building blocks making up the artificial receptor caused significant changes in the degree to which GM1 enhanced binding of cholera toxin.

### Discussion of Examples 4-6

We have previously demonstrated that an array of working artificial receptors bind to a protein target in a manner which is complementary to the specific environment presented by each region of the proteins surface topology. Thus the pattern of binding of a protein target to an array of working artificial receptors describes the proteins surface topology; including surface structures which participate in e.g., protein˜small molecule, protein˜peptide, protein-protein, protein˜carbohydrate, protein˜DNA, etc. interactions. It is thus possible to use the binding of a selected protein to a working artificial receptor array to characterize these protein˜small molecule, protein˜peptide, protein-protein, protein˜carbohydrate, protein˜DNA, etc. interactions. Moreover, it is possible to utilize the protein to array interactions to define “leads” for the disruption of these interactions.

Cholera Toxin B sub-unit binds to GM1 on the cell surface. Studies to identify competitors to this binding event have shown that competitors to the cholera toxin: GM1 binding interaction (binding site) can utilize both a sugar and an alkyl/aromatic functionality (Pickens, et al., Chemistry and Biology, vol. 9, pp 215-224 (2002)). We have previously demonstrated that fluorescently labeled Cholera Toxin B sub-unit binds to arrays of the present artificial receptors to give a defined binding pattern which reflects cholera toxin B's surface topology. For this study, we sought to demonstrate that the binding of the cholera toxin to at least some members of the array could be disrupted using cholera toxin's natural ligand, GM1.

The results presented in the figures clearly demonstrate that these goals have been achieved. Specifically, competition between the GM1 OS pentasaccharide or GM1 and an artificial receptor array for cholera binding clearly gave a binding pattern which was distinct from the cholera binding pattern control. Moreover, these results demonstrated the complementarity between several of the working artificial receptors which contained a naphthyl moiety when compared to working artificial receptors which only contained phenyl functionality. These results are in keeping with the active site competition studies in Pickens, et al. and indicate that the naphthyl and phenyl derivatives represent good mimics/probes for the cholera to GM1 interaction. The specificity of these interactions was demonstrated by the observation that the change of a single building block out of 4 in a combination of 4 building blocks system changed a non-competitive to a significantly competitive environment. These results also indicated that selected working artificial receptors can be used to develop a high-throughput screen for the further evaluation of the cholera:GM1 interaction.

Additionally, we sought to demonstrate that an affinity support/membrane mimic could be prepared by pre-incubating an array of artificial receptors with GM1 which would then bind/capture cholera toxin in a binding pattern which could be used to select a working artificial receptor(s) for, for example, the high-throughput screen of lead compounds which will disrupt the “cholera:membrane˜GM1 mimic”. The GM1 pre-incubation studies clearly demonstrated that several of the working artificial receptors which were poor cholera binders significantly increased their cholera binding, presumably through an affinity interaction between the cholera toxin and both the immobilized GM1 pentasaccharide moiety and the working artificial receptor building block environment.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration. The phrase “adapted and configured” can be used interchangeably with other similar phrases such as arranged and configured, constructed and arranged, adapted, constructed, manufactured and arranged, and the like.

All publications and patent applications in this specification are indicative of the level of ordinary skill in the art to which this invention pertains. All publications, patent applications, and patents referenced herein are incorporated by reference to the same extent as if each individual publication, patent application, or patent was specifically and individually indicated by reference.

The invention has been described with reference to various specific and preferred embodiments and techniques. However, it should be understood that many variations and modifications may be made while remaining within the spirit and scope of the invention.

1. A method of adhering components together comprising:
disposing a first artificial receptor on a first component, wherein the first artificial receptor comprises a plurality of building blocks coupled to the first component, wherein the first artificial receptor is known to having binding affinity for a second component; and
allowing the artificial receptor to bind to the second component.

2. The method of claim 1, wherein the first component and the second component comprise nano-scale components.

3. The method of claim 2, wherein the first component comprises an item selected from the group consisting of a sheet, lattice, shell, wire, chain, ring, icosahedron, square pyramid, tetrahedron, staircase structure, sphere, tube, and helix.

4. The method of claim 1, further comprising disposing a second artificial receptor on the second component, wherein the second artificial receptor comprises a plurality of building blocks coupled to the second component, wherein the second artificial receptor is known to having binding affinity for the first artificial receptor.

5. A device comprising:
a first component;
a second component; and
a first binding pair of artificial receptors comprising a first artificial receptor and a second artificial receptor,
wherein the first and second artificial receptors each comprise a plurality of building blocks, wherein the first artificial receptor is known to having binding affinity for the second artificial receptor; wherein the first artificial receptor is disposed on the first component and the second artificial receptor is disposed on the second component; the first component adhered to the second component via the first binding pair.

6. The device of claim 5, further comprising:
a third component; and
a second binding pair of artificial receptors comprising a third artificial receptor and a fourth artificial receptor;
wherein the third and the fourth artificial receptors each comprise a plurality of building blocks, wherein the third artificial receptor is known to having binding affinity for the fourth artificial receptor; wherein the third artificial receptor is disposed on the first component and the fourth artificial receptor is disposed on the third component; the first component adhered to the third component via the second binding pair of artificial receptors.

7. The device of claim 5, the device comprising a sheet, lattice, shell, wire, chain, ring, icosahedron, square pyramid, tetrahedron, staircase structure, sphere, tube, or helix.

8. The device of claim 5, wherein the first component and the second component comprise nanotubes.

9. An agent delivery device comprising:
a capsule;
an active agent, wherein the active agent is disposed within the capsule; and
an artificial receptor disposed on the capsule, comprising a plurality of building blocks coupled to the capsule, wherein the artificial receptor is known to have binding affinity for a target ligand.

10. The agent delivery device of claim 9, comprising a temperature-sensitive polymer and a metal nanoshell.

11. The agent delivery device of claim 9, the capsule comprising a polyelectrolyte shell.

12. The agent delivery device of claim 9, wherein the active agent is selected from the group consisting of thrombin inhibitors, antithrombogenic agents, thrombolytic agents, fibrinolytic agents, anticoagulants, anti-platelet agents, vasospasm inhibitors, calcium channel blockers, steroids, vasodilators, anti-hypertensive agents, antimicrobial agents, antibiotics, antibacterial agents, antiparasite and/or antiprotozoal solutes, antiseptics, antifungals, angiogenic agents, anti-angiogenic agents, inhibitors of surface glycoprotein receptors, antimitotics, microtubule inhibitors, antisecretory agents, actin inhibitors, remodeling inhibitors, antisense nucleotides, anti-metabolites, miotic agents, anti-proliferatives, anticancer chemotherapeutic agents, anti-neoplastic agents, antipolymerases, antivirals, anti-AIDS substances, anti-inflammatory steroids or non-steroidal anti-inflammatory agents, analgesics, antipyretics, immunosuppressive agents, immunomodulators, growth hormone antagonists, growth factors, radiotherapeutic agents, peptides, proteins, enzymes, extracellular matrix components, ACE inhibitors, free radical scavengers, chelators, anti-oxidants, photodynamic therapy agents, gene therapy agents, anesthetics, immunotoxins, neurotoxins, opioids, dopamine agonists, hypnotics, antihistamines, tranquilizers, anticonvulsants, muscle relaxants and anti-Parkinson substances, antispasmodics and muscle contractants, anticholinergics, ophthalmic agents, antiglaucoma solutes, prostaglandins, antidepressants, antipsychotic substances, neurotransmitters, anti-emetics, imaging agents, specific targeting agents, and cell response modifiers.

13. The agent delivery device of claim 9, wherein the target ligand comprises a protein specific to a carcinoma cell.

14. The agent delivery device of claim 9, wherein the target ligand comprises a molecule expressed by a microbe.

15. An agent delivery device comprising:
a nanotube;
an active agent disposed on the nanotube;
a cap disposed on the nanotube having an open position and a closed position, wherein the active agent is prevented from vacating the nanotube when the cap is in the closed position; and
an artificial receptor disposed on the cap, comprising a plurality of building blocks coupled to the cap,
wherein the artificial receptor has a binding affinity for the nanotube that can be overcome by a release compound, wherein the cap is in the closed position when the artificial receptor is bound to the nanotube.

16. A detection device comprising:
a magnetic particle; and
an artificial receptor disposed on the magnetic particle, the artificial receptor comprising a plurality of building blocks coupled to the magnetic particle, wherein the artificial receptor is known to have binding affinity for a target ligand.

17. The detection device of claim 16, the magnetic particle comprising ferrite.

18. The detection device of claim 16, the target ligand comprising a drug of abuse, a disease marker, polynucleotide, a polypeptide, a microbe, a contaminant, or a small molecule.

19. A detection device comprising:
a quantum dot; and
an artificial receptor disposed on the quantum dot, the artificial receptor comprising a plurality of building blocks coupled to the quantum dot, wherein the artificial receptor is known to have binding affinity for a target ligand.

20. The detection device of claim 19, the quantum dot comprising silicon.

21. The detection device of claim 19, the target ligand comprising a drug of abuse, a disease marker, polynucleotide, a polypeptide, a microbe, a contaminant, or a small molecule.

22. A detection device comprising:
a plurality of first particles;
a plurality of first artificial receptors disposed on the first particles, the first artificial receptors comprising a plurality of building blocks coupled to the first particles, wherein the first artificial receptors are known to have binding affinity for a first part of a target ligand;
a plurality of second particles, and
a plurality of second artificial receptors disposed on the second particles, the second artificial receptors comprising a plurality of building blocks coupled to the second particles, wherein the second artificial receptor is known to have binding affinity for a second part of a target ligand;
wherein the first particles and the second particles aggregate in the present of the target ligand.

23. The detection device of claim 22, the particle comprising silicon.

24. The detection device of claim 22, the particle comprising a quantum dot.

25. The detection device of claim 22, the target ligand comprising a drug of abuse, a disease marker, polynucleotide, a polypeptide, a microbe, a contaminant, or a small molecule.

26. A detection device comprising:
a cantilever; and
an artificial receptor disposed on the cantilever, the artificial receptor comprising a plurality of building blocks coupled to the cantilever, wherein the artificial receptor is known to have binding affinity for a target ligand.

27. The detection device of claim 26 comprising a plurality of cantilevers.

28. The detection device of claim 26, the cantilever comprising silicon.

29. The detection device of claim 26, the target ligand comprising a drug of abuse, a disease marker, polynucleotide, a polypeptide, a microbe, a contaminant, or a small molecule.

30. A detection device comprising:
a substrate; and
an artificial receptor disposed on the substrate; the artificial receptor comprising a plurality of building blocks coupled to the substrate, wherein the artificial receptor is known to have binding affinity for a target ligand;
wherein the substrate has electrical properties that change when the target ligand is bound to the artificial receptor.

31. The detection device of claim 30, wherein the substrate comprises a nanowire.

32. The detection device of claim 31, wherein the substrate comprises a nanowire field effect transistor.

33. The detection device of claim 30, wherein the substrate comprises a nanotube.

34. The detection device of claim 30, wherein the conductance of the substrate changes when the target ligand is bound to the artificial receptor.

35. The detection device of claim 30, wherein the artificial receptor is covalently bound to the substrate.

36. The detection device of claim 30, the target ligand comprising a drug of abuse, a disease marker, polynucleotide, a polypeptide, a microbe, a contaminant, or a small molecule.

37. A device comprising:
a first nanotube tip and a second nanotube tip;
a first artificial receptor disposed on the first nanotube tip, the first artificial receptor comprising a plurality of building blocks coupled to the first nanotube tip, wherein the first artificial receptor is known to have binding affinity for a target ligand;
a second artificial receptor disposed on the second nanotube tip, the second artificial receptor comprising a plurality of building blocks coupled to the second nanotube tip, wherein the second artificial receptor is known to have binding affinity for the target ligand; and
a first electrode and a second electrode, wherein the first electrode is in electrical communication with the first nanotube tip and the second electrode is in electrical communication with the second nanotube tip.

38. The device of claim 37, wherein the first artificial receptor and the second artificial receptor are the same.

39. A device for selective removal of a target component comprising:
a substrate; and
an artificial receptor disposed on the substrate, the artificial receptor comprising a plurality of building blocks coupled to the substrate, wherein the artificial receptor is known to have binding affinity for the target component; wherein the substrate enhances selective removal of the target component.

40. The device of claim 39, the substrate comprising a liposome.

41. The device of claim 39, the substrate comprising a magnetic bead.

42. The device of claim 39, the target component comprising a lipophilic agent.

43. The device of claim 39, the target component comprising a drug of abuse.

44. The device of claim 39, the target component comprising a biological material.

45. The device of claim 39, the target component comprising lipopolysaccharide.
